(19) United States
(12) Patent Application Publication
(10) Pub. No.: US 2026/0071234 A1
(43) Pub. Date: Mar. 12, 2026

(54) METHODS AND COMPOSITIONS FOR HEMATOPOIETIC STEM CELL ENHANCEMENT

(71) Applicant: THE REGENTS OF THE UNIVERSITY OF CALIFORNIA, Oakland, CA (US)

(72) Inventors: Hanna MIKKOLA, Los Angeles, CA (US); Júlia AGUADÉ-GORGORIÓ, Los Angeles, CA (US)

(73) Assignee: THE REGENTS OF THE UNIVERSITY OF CALIFORNIA, Oakland, CA (US)

(21) Appl. No.: 19/106,164

(22) PCT Filed: Aug. 25, 2023
(86) PCT No.: PCT/US2023/072940

§ 371 Date: Feb. 24, 2025

Related U.S. Application Data
(60) Provisional application No. 63/401,011, filed on Aug. 25, 2022.
(60) Provisional application No. 63/472,732, filed on Jun. 13, 2023.

(51) Int. Cl. C12N 15/86; C12N 5/0789

(52) U.S. Cl. CPC C12N 15/86; C12N 5/0647; C12N 2500/90; C12N 2501/125; C12N 2501/145; C12N 2501/26; C12N 2501/998; C12N 2740/15043

(57) ABSTRACT

Disclosed are methods and compositions for inhibiting endocytosis and enhancing function of hematopoietic stem cells. Certain aspects are directed to increasing MYCT1 activity or expression to improve hematopoietic stem cell self-renewal and/or engraftment ability.

This application claims priority of U.S. Provisional Patent Application No. 63/401,011, filed Aug. 25, 2022 and U.S. Provisional Patent Application No. 63/472,732, filed Jun. 13, 2023, both of which are hereby incorporated by reference in their entirety.

This invention was made with government support under DK100959, and HL162408 awarded by the National Institutes of Health. The government has certain rights in the invention.

### BACKGROUND

### I. Technical Field

Aspects of this invention relate generally to the fields of hematology, molecular biology, and medicine.

### II. Background

Human hematopoietic stem cell (HSC) sustain life-long blood production and can reconstitute the entire hematopoietic system after transplantation. However, the biological processes governing HSC self-renewal and engraftment ability are still poorly understood, and cannot be recapitulated ex vivo to facilitate robust human HSC expansion. Despite recent advances, the ability to expand functional human HSCs in culture for therapeutic use is still limited. There exists a need for identification of factors critical for HSC self-renewal and engraftment capability, as well as for methods and compositions for enhancing these capabilities.

### SUMMARY

Disclosed herein, in some aspects, is a method for enhancing hematopoietic stem cell function, the method comprising introducing a nucleic acid encoding MYCT1 into a hematopoietic stem cell. In some aspects, the hematopoietic stem cell is a human hematopoietic stem cell. It is specifically contemplated that other cell types may be excluded from certain aspects herein. In some aspects, the method further comprises culturing the hematopoietic stem cell for at least, at most, or approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 hours, or more, or any range derivable therein. In some aspects, the method further comprises introducing the hematopoietic stem cell into a subject. In some aspects, the subject is a human subject. In some aspects, the hematopoietic stem cell engrafts into bone marrow of the subject. In some aspects, the method further comprises differentiating the hematopoietic stem cell into a blood cell. In some aspects, the nucleic acid molecule is a plasmid. In some aspects, the nucleic acid molecule is a vector. In some aspects, the vector is a viral vector. In some aspects, the viral vector is a recombinant adeno-associated viral vector, a recombinant adenoviral vector, a recombinant lentiviral vector, or a recombinant retroviral vector. In some aspects, the nucleic acid is a DNA molecule. In some aspects, the nucleic acid is an mRNA molecule. In some aspects, introducing the nucleic acid comprises transfection. In some aspects, the nucleic acid molecule does not integrate into the genome of the hematopoietic stem cell. In some aspects, the nucleic acid molecule integrates into the genome of the hematopoietic stem cell. In some aspects, the method comprises introducing a nucleic acid molecule encoding MYCT1 into each of a population of hematopoietic stem cells. In some aspects, the method further comprises introducing a nucleic acid molecule encoding MLLT3 into the hematopoietic stem cell.

Also disclosed herein, in some aspects, is a modified hematopoietic stem cell having increased expression or activity of MYCT1 relative to an unmodified hematopoietic stem cell. The modified hematopoietic stem cell may be modified by any method disclosed herein. In some aspects, the hematopoietic stem cell comprises an exogenous nucleic acid molecule encoding MYCT1. In some aspects, the hematopoietic stem cell comprises an exogenous nucleic acid molecule encoding MYCT1. In some aspects, the hematopoietic stem cell comprises a genetic modification that increases the activity of MYCT1.

Further disclosed herein, in some aspects, is a method for enhancing hematopoietic stem cell function, the method comprising subjecting a hematopoietic stem cell to conditions sufficient to control the rate of endocytosis. In some aspects, the conditions comprise administration of an endocytosis inhibitor. In some aspects, the conditions increase expression or activity of MYCT1. In some aspects, the conditions comprise introducing a nucleic acid encoding MYCT1 into a hematopoietic stem cell. In some aspects, the nucleic acid molecule is a plasmid. In some aspects, the nucleic acid molecule is a vector. In some aspects, the vector is a viral vector. In some aspects, the viral vector is a recombinant adeno-associated viral vector, a recombinant adenoviral vector, a recombinant lentiviral vector, or a recombinant retroviral vector. In some aspects, the nucleic acid is a DNA molecule. In some aspects, the nucleic acid is an mRNA molecule. In some aspects, introducing the nucleic acid comprises transfection. In some aspects, the nucleic acid molecule does not integrate into the genome of the hematopoietic stem cell. In some aspects, the nucleic acid molecule integrates into the genome of the hematopoietic stem cell. In some aspects, the method comprises introducing a nucleic acid molecule encoding MYCT1 into each of a population of hematopoietic stem cells. In some aspects, the method further comprises introducing a nucleic acid molecule encoding MLLT3 into the hematopoietic stem cell.

Also disclosed are methods for treating a subject, methods of administering a cellular therapy a subject, methods of increasing MYCT1 in a cellular population (such as a population of HSCs) in a subject. The methods can comprise 1, 2, 3, 4, 5 or more steps including any of the following steps: introducing a nucleic acid encoding MYCT1 into a hematopoietic stem cell, introducing a MYCT1 gene product into a hematopoietic stem cell, culturing a hematopoietic stem cell (including the hematopoietic stem cell with an introduced MYCT1 nucleic acid and/or gene product), introducing the hematopoietic stem cell into a subject, administering a therapeutic composition (which can comprise any hematopoietic stem cell, nucleic acid, and/or vector disclosed herein) to a subject, subjecting a hematopoietic stem cell to conditions sufficient to inhibit endocytosis, or a combination thereof.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-L. MYCT1 is functionally required for human HSC expansion and engraftment. a MYCT1 gene expression from RNAseq on sorted populations from human 5 to 6 week aorta-gonad-mesonephros (AGM), second trimester fetal liver (FL), and cord blood (CB). b Single cell RNAseq scorecard dotplot for selected HSC genes in uncultured sorted CB HSPCs (CD34+CD38−CD90+) grouped based on HLF and MYCT1 expression. c MYCT1 expression form published datasets of GPI80+ and − subpopulations of human FL HSPC (Prashad et al) and d ITGA3+ and − subpopulations of UM171-expanded human CB HSPCs (Tomellini et al). e Relative MYCT1 expression from RNAseq in sorted CB HSPCs (CD34+CD38−CD90+) uncultured and after 5 days in culture (n=2 independent CB). f Relative MYCT1 expression from qPCR in sorted CB HSPCs uncultured and after 2 weeks in culture (n=3 independent CB). g Experiment outline for ex vivo expansion and transplantation assays with MYCT1 knockdown HSPCs. h Relative MYCT1 expression from qPCR in sorted CB HSPCs 72 hours after transduction with lentiviral control or MYCT1 knockdown shRNAs. i-j Fold expansion of HSPCs (i) or total live cells (j) from FL (CD34+CD38−CD90+GPI80+) or CB (CD34+CD90+) transduced with control or MYCT1 shRNAs (n=2 for FL and n=3 for CB). k Quantification and representative FACS plots of human engraftment (human CD45) in bone marrow at 6, 12 and 24 weeks after transplantation, and spleen and blood 24 weeks after transplantation in NSG mice of equal number of sorted HSPCs transduced with control of MYCT1 shRNA (n=4 or 5 mice for FL, n=5 or 6 for CB). l Fold expansion of E4EC and HUVEC cells 15 days after transduction with control or MYCT1 shRNAs (n=5 independent experiments for E4EC and n=3 for HUVEC).

FIG. 2A-E. MYCT1 is expressed in HSC, EC, and megakaryocytes, and silenced in culture. a MYCT1 gene expression from RNAseq on sorted populations from human placenta (PL) and yolk sac (YS) from 4-5 week embryos, and adult bone marrow (BM). b MYCT1 expression in purified populations of human hematopoietic cells from published datasets (DMAP, Novershtern 2011). c TSNE plots of human 4-5 week AGM from published datasets (Calvanese 2022) with endothelial cells (CDH5), HSC (HLF), megakaryocytes/platelets (PF4), and MYCT1 expression highlighted. d UCSC genome browser tracks showing ChIP-seq of epigenetic marks for MYCT1 and MECOM genomic regions in sorted FL HSPCs before and after 4 weeks in culture with control or MLLT3 overexpression. e Relative gene expression for MYCT1 and MECOM in the same conditions as (d). d,e are from published datasets (Calvanese 2019).

FIG. 3A-F. MYCT1 is essential for human HSC. a Western blot in KG1 cells overexpressing MYCT1-V5 (OE) after additional transduction with control or MYCT1 shRNAs. b,c FACS analysis (b) and quantification (c) of phenotypic HSPCs 2 weeks after transduction and sorting of FL (CD34+CD38−CD90+GPI80+) or CB (CD34+CD90+) HSPCs control or MYCT1 shRNAs (representative plots from n=2 for FL and n=3 for CB independent experiments). d FACS plots of the FL and CB HSPCs sorted for transplantation after transduction with control or MYCT1 shRNA. The red gate represents the sorted HSPCs (CD34+CD38−CD90+ and GPI80+ in case of FL). Equal number or sorted cells were used for the transplantation experiments in FIG. 1. e,f FACS analysis showing hematopoietic reconstitution (human CD45) and multilineage analysis in bone marrow from NSG mice 24 weeks after transplantation of control FL (e) or CB (f) HSPCs (representative plots from (n=5 mice for FL, n=6 for CB).

FIG. 4A-B. MYCT1 is essential for the expansion of human E4EC. a,b Relative MYCT1 expression from qPCR 72 hours after transduction (a) and representative images 6 and 14 days after transduction of E4ECs with control or MYCT1 shRNAs (b).

FIG. 5A-K. MYCT1 governs HSC hallmarks and proliferation. a Experiment outline for scRNAseq in sorted HSPCs uncultured and after 5 days in culture with control, MYCT1 knockdown or MYCT1 overexpression. b,c TSNE plots for all sequenced cells (b) and HLF+ cells in all cultured samples (c). d Proportion of HLF+ cells in each HSPC sample. e-g Dot plots for selected genes (e,f) or module scores (g) in HLF+ cells in control, KD, OE, and uncultured samples. h FACS histograms and quantification of proliferation analysis by dye dilution (CellTrace) in CB HSPCs transduced with control or MYCT1 shRNAs at the time of labelling and after 48 hours (n=3 independent experiments). i-k Percentage of cells that undergo 1 to 4 divisions (i), non-dividing cells (j), and cell death (k) determined by individual monitoring of single sorted HSPCs every 24 hours after transduction with control or MYCT1 shRNAs (n=4 independent experiments).

FIG. 6A-D. MYCT1 governs HSC transcriptomic signatures. a FACS plots of the CB HSPCs sorted for scRNAseq uncultured, or after transduction with control, MYCT1 shRNA or OE. The CD38− cells are shown and the red gate represents the sorted HSPCs (CD34+CD38−CD90+ and GFP+ for the transduced cells). b Number of HSPCs and HLF+ cells sequenced for scRNAseq for each sample. c,d Dot plots for selected genes that become downregulated (c) or upregulated (d) in HLF+ HSC after MYCT1 knockdown, and restored by MYCT1 OE.

FIG. 7A-E. MYCT1 controls HSC proliferation. a FACS analysis and quantification of cell cycle distribution by EdU incorporation and DNA amount (FxCycle) in CB HSPCs (CD34+EPCR+) 72 hours after transduction with control or MYCT1 shRNAs. b-d Percentage of cells that undergo 1 to 4 divisions (b), non-dividing cells (c), and cell death (d) determined by individual monitoring of single sorted HSPCs every 24 hours after transduction with control or MYCT1 OE (n=3 independent experiments). e FACS analysis and quantification of cell cycle distribution by BrdU incorporation and DNA amount (7AAD) in E4ECs 96 hours after transduction with control or MYCT1 shRNA.

FIG. 8A-H. MYCT1 is an endosomal protein interacting with vesicle trafficking and signaling components. a Cell fractionation and western blot of KG1 cells overexpressing MYCT1-V5, and quantification of the relative MYCT1 amount in each fraction (n=3 independent experiments). b Immunofluorescence of KG1 cells overexpressing MYCT1-V5 stained for V5, DAPI, and endosomal markers (Clathrin, Rab5, Rab7, Rab11) and a mitochondrial marker (HSP60) as negative control. Colocalization channel (coloc) shows areas positive for V5 and each marker. Scale bars=10 μm. Representative images of n=3 independent experiments. c Quantification of percentage of MYCT1 colocalization with each marker. Colocalization analysis was performed on 9-17 images per condition, corresponding to n=3 independent experiments. P value determined by Mann Whitney test in relation to the negative control (HSP60). d,e Immunofluorescence and quantification of colocalization as in (b,c) of HSPCs transduced with MYCT1-V5 and cultured for 5 days. f Model of MYCT1 structure, localization, and topology. g Heatmap of MYCT1 interactors detected by immunoprecipitation coupled with high-sensitivity mass spectrometry in KG1 or E4EC with control or MYCT1-V5 overexpression. Scale depicts the number of detected peptides for each protein. Colours indicate protein categories. N=3 independent experiments in duplicates for KG1, n=1 experiment in duplicates for E4EC. h Heatmaps depicting the MYCT1 interactors detected in KG1 and E4EC and their expression from RNAseq in KG1, E4EC and sorted ECs and HSPCs from embryonic tissues (AGM, YS=yolk sac, PL=placenta), second trimester FL, CB, and post-natal bone marrow (BM).

FIG. 9A-I. MYCT1 structure, localization, and interactome. a Schematic depiction of MYCT1 transmembrane domains (TM1 and 2) and nuclear localization signal (NLS). b Histogram of probability of membrane topology for MYCT1 protein. c FACS analysis of HSC surface markers CD34 and CD90 in KG1 and human CB HSPCs. d Gene expression (RNAseq) for selected HSC genes in KG1 and K562 cell lines from the Cancer Cell Line Encyclopaedia. e Immunohistochemistry of MYCT1. f Western blot for immunoprecipitation using anti-V5 antibody in KG1 transduced with control or overexpression of V5-tagged MYCT1. g Venn diagram of the number of interactors detected in KG1 and E4EC. h Interaction network (functional and physical) of MYCT1 interactors identified in KG1 generated using STRING (Szklarczyk et al). i Heatmaps of −Log P values of GO, Reactome (REAC), KEGG, and WikiPathway (WP) terms for the MYCT1 interactome in KG1 and E4EC.

FIG. 10A-N. MYCT1 controls endocytosis and environmental sensing and governs HSC stemness. a Quantification and representative FACS histogram of internalized fluorescent dextran in CB HSPCs cultured for 5 days transduced with control, MYCT1 KD or OE (n=3 or 4 independent experiments, P values determined by two-tailed paired t test). b Predicted relative kinase activity (Kinase-Substrate Enrichment Analysis, KSEA) and c enriched pathways and perturbations (post-translational modification enrichment analysis, PTM-SEA) from global phospho-proteomic profiling of E4ECs 72 hours after transduction with MYCT1 shRNAs compared to control. N=1 experiment in duplicates. d-f Representative western blot of phospho-AKT in CB HSPCs transduced with control or MYCT1 shRNAs starved overnight and after 30 minutes and 3 hours of stimulation with SCF (d) or under normal culture conditions (e), and the respective quantification (f). P calculated by Ratio Paired t test. g Dot plot for signaling module scores in HLF+ HSC transduced with control, MYCT1 KD or OE after 5 days in culture. h Internalized fluorescent dextran in different subpopulations of CB HSPCs at different time points in culture (n=4 independent experiments, P values determined by 2-tailed unpaired t test). i Quantification and representative histogram of internalized fluorescent dextran in CB HSPCs (CD34+CD90+) cultured for 8-12 days transduced with control, MYCT1 KD or OE (n=6 independent experiments). P values determined by two-tailed paired t test. j,k Total number of colonies (j) and number of granulocyte/macrophage (GM), erythroid (CFU/BFU-E) and mixed (granulocyte, erythroid, monocyte and macrophage, GEMM) colonies (k) formed after plating in enriched methylcellulose equal number of CB HSPCs transduced with control or MYCT1 OE, cultured for 5 days, and sorted (CD34+CD38−CD90+GFP+). l,m Quantification of human engraftment (human CD45) in bone marrow (l) and percentage of engrafted mice (m) at 12 weeks after transplantation of equal number of sorted CB HSPCs (CD34+CD38−CD90+GFP+) at day 5 transduced with control of MYCT1 OE into NBSGW mice (n=18 mice per group in total. N=5 for 2500 HSPCs and n=6 for 500 HSPCS transplanted into female mice, n=7 for 500 HSPCs transplanted into male mice. P values determined by two-tailed paired t test. n Calculation of stem cell frequency and P value from limiting dilution assay using ELDA software from the transplantation of 500 and 2500 HSPC into female NBSGW mice.

FIG. 11A-F. MYCT1 controls endocytosis in endothelial cells and human HSPCs. a-d Quantification and representative FACS histogram of internalized fluorescent dextran (a), transferrin (b), or transferrin receptor (TFRC) (c) in E4EC 72-96 hours after transduction with control or MYCT1 shRNAs. d Quantification and representative histogram of internalized fluorescent dextran as in (a) after pre-treatment with 10 μM chlorpromazine (chpz). e,f Quantification and representative FACS histogram of transferrin internalization (e) or dextran internalization after pre-treatment with 10 μM chlorpromazine (chpz) or methyl-β-cyclodextin (MbCD) 1 mM (f) in CB HSPCs cultured for 5 days transduced with control, MYCT1 KD or OE. For a-f n=3 or 4 independent experiments. P values are determined by two-tailed paired (a,b,e) or unpaired (c,d,f) t test.

FIG. 12A-I. MYCT1 governs environmental sensing. (a) Experiment and analysis outline for pospho-proteomic profiling in E4EC 72 h after transduction with control or MYCT1 shRNAs. (b) Log 2 fold change between shRNA and control for all identified phosphorylated protein sites in the duplicate samples. (c) Pearson correlation between the two different shRNAs. d,e −Log P values for selected WikiPathway (WP) (d) and Reactome (REAC) (e) terms for the proteins with increased (red) or decreased (blue) phosphorylation after transduction with MYCT1 shRNA compared to control. (f-i) Western blot and respective quantification of phospho-AKT and phospho-ERK in E4ECs transduced with control or MYCT1 shRNAs after overnight starvation and subsequent stimulation with complete media (containing serum, FGF, EGF and IGF) for 30 minutes, 1 and 3 hours (f,g) or under normal culture conditions (h,i) (n=3 independent experiments).

FIG. 13A-C. MYCT1 in HSPCS. (a,b) MYCT1 is enriched in self-renewing HSPCs. (c) MYCT1 is one of the most downregulated genes in cultured human HSPCs.

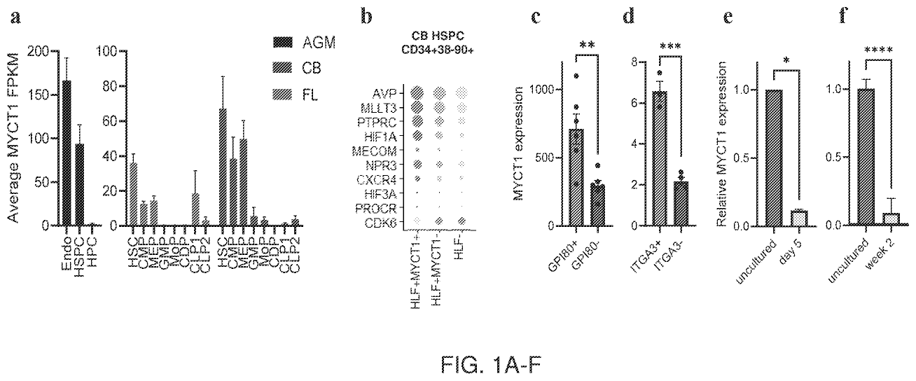

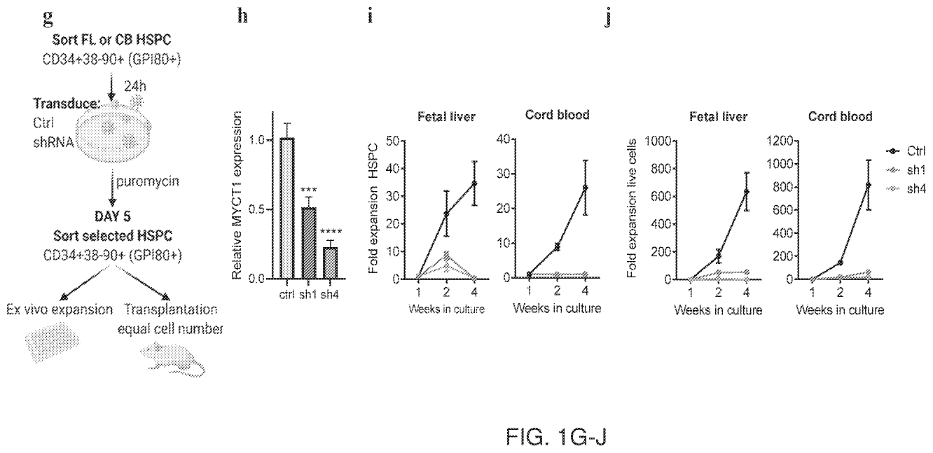

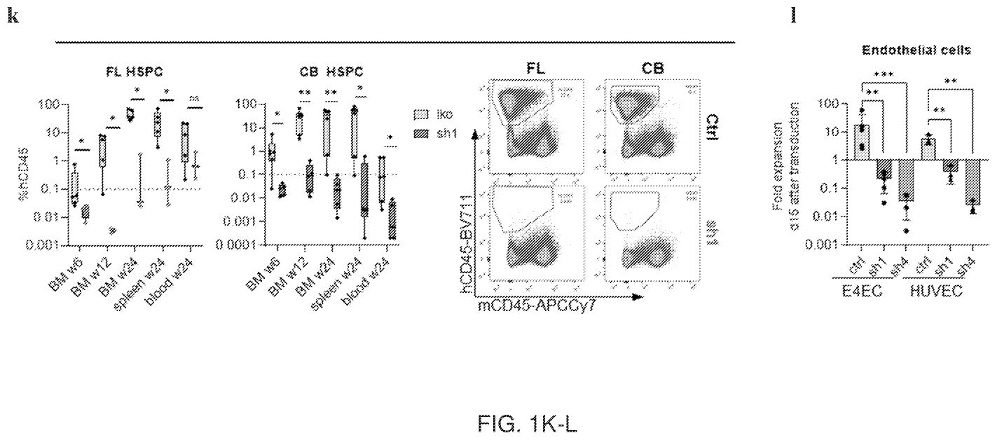

FIG. 1A-B4. MYCT1 is required for human HSC function. (a) MYCT1 knockdown in human CB HSPC impairs the expansion of LT-HSC, and (b) engraftment after transplantation into NSG mice.

FIG. 15. MYCT1 governs HSC programs and hallmarks of HSC functional competence. scRNAseq on sorted HSPC transduced with control, MYCT1 overexpression (OE), or knockdown (KD), and uncultured HSPC. Module score analysis on the HLF+ cells revealed dysregulation of HSC “stemness” programs by MYCT1 KD, which were restored by MYCT1 OE.

FIG. 16. Restoring MYCT1 expression improves phenotypes, including self-renewal and engraftment ability, in culture and transplantability of human HSC. (a) MYCT1 overexpression (OE) increases the number of human LT-HSC during ex vivo culture. (b) Transplantation of MYCT1 OE HSPCs (CD34+CD38−CD90+) into NBSGW mice 96 hours after transduction or (c) their progeny after 15 days results in improved human reconstitution 12 weeks after transplantation compared to control cells.

FIG. 17A-B. MYCT1 is an endosomal protein that interacts with vesicle trafficking and signaling machinery. MYCT1 localizes in the membrane of endosomes and interacts with components of vesicle trafficking and signaling machinery. (a) Immunofluorescence of MYCT1-V5 in KG1 shows that MYCT1 localizes in different endosomal subtypes. (b) Immunoprecipitation coupled with mass spectrometry revealed that MYCT1 interacts with vesicle trafficking proteins, signaling components, cell adhesion proteins and protein degradation machinery. Selected MYCT1 interactors are shown.

FIG. 18A-D. MYCT1 controls endocytosis and environmental sensing. Loss of MYCT1 causes increase dendocytosis and environmental hypersensitivity. (a) MYCT1 KD increases dendocytosis in human CBHSPC, while overexpression (OE) decreased it. (b) Phospho-proteomics analysis of control and MYCT1 KD human endothelial cells shows a widespread activation of signaling pathways, including increased response to the cytokines in the culture media (EGF, IGF, FGF). (c) Western blot shows hyperactivation of AKT signaling in MYCT1 KD endothelial cells and (d) confirmed increased AKT phosphorylation in MYCT1 KD human HSPCs, while MYCT1 OE decreased it.

FIG. 19. Soluplus human HSC expansion culture improves MYCT1 levels. scRNAseq analysis of the Sakurai et al dataset (HLF+).

FIG. 20. Schematic for environmental sensing in human hematopoietic stem cells.

FIG. 21A-G. MYCT1 is critical for human HSPC ex vivo expansion and engraftment ability. (a) MYCT1 gene expression across human hematopoietic ontogeny from RNAseq on sorted populations from human 5-6 weeks aorta-gonad-mesonephros (AGM), placenta (PL), yolk sac (YS), second trimester foetal liver (FL), cord blood (CB), and adult bone marrow (ABM). n=6 (AGM), n=4 (PL), n=3 (YS, FL, CB, ABM) donors per tissue, mean±s.e.m. (b) MYCT1 expression in highly purified human FL GPI80+ and − HSPC (CD34+CD38−CD90+) subsets²⁹, and UM171-expanded cord blood ITGA3+ and − HSPC (CD90+EPCR+CD133+CD34+CD45RA−) subsets¹⁸. n=3 and 4 replicates respectively, mean±s.e.m, two-tailed paired t-test. P values from left to right: 0.0096, 0.0002. (c) qRT-PCR quantification of relative MYCT1 expression in CB HSPCs (CD34+CD38−CD90+) sorted before and after (2, 5 and 15 days) culture. n=3 experiments, mean±s.e.m, two-tailed paired t-test. P values from left to right: 0.001, 0.0006, 0.0004. (d) Experimental outline to evaluate the effects of MYCT1 knockdown (KD) on human CB HSPC ex vivo expansion and in vivo engraftment ability. (e) Fold expansion of all live cells, total HSPC fraction (CD34+CD38−CD90+CD45RA−CD90+), and immunophenotypical long-term HSC (LT-HSC, CD34+CD38−CD90+CD45RA−CD90+EPCR+ITGA3+) from CB transduced with control or MYCT1 KD. n=4 experiments, mean±s.e.m., two-tailed Mann-Whitney test. P values from left to right KD1 all cells: 0.041, <0.0001, <0.0001, <0.0001; KD2 all cells: 0.0016, 0.0016, 0.0007, 0.0007; KD1 total HSPC: 0.0055, 0.0022, 0.0044, 0.0004; KD2 total HSPC: 0.0062, 0.0031, 0.0027, 0.0027; KD1 LT-HSC: 0.0252, 0.0002, <0.0001, 0.0008; KD2 LT-HSC: 0.0186, 0.0016, 0.0007, 0.0083. (f) FACS plots showing control or MYCT1 KD CB HSPCs sorted for transplantation 72 hours after transduction. The black gate indicates the population of CB HSPCs (CD34+CD38−CD90+) that were sorted and transplanted. 5,000 sorted control or KD HSPCs were transplanted per mouse. (g) Representative FACS plots and quantification of human hematopoietic engraftment (human CD45+) and differentiated populations in NSG mice transplanted with equal number of sorted HSPCs transduced with control or MYCT1 KD. In FACS plots, differentiated cells are quantified as percentage within total hCD45+ cells. Quantification shows bone marrow at 6, 12 and 24 weeks after transplantation, and spleen and blood 24 weeks after transplantation. n=6 (control) and n=5 (MYCT1 KD) mice per group, mean±s.e.m., two-tailed Mann-Whitney test.

FIG. 22A-G. MYCT1 governs regulatory programs associated with human HSC functional competence. (a) Experimental outline for scRNAseq analysis evaluating the effects of MYCT1 knockdown (KD) and MYCT1 overexpression (OE) in CB HSPCs 72 hours after transduction (4 days in culture). CB HSPCs were isolated freshly or 72 hours after transduction. (b) Single cell RNAseq dot plot documenting the expression of selected human HSC signature genes from multiple datasets^{17,31-34}, as well as CDK6, that are significantly differentially expressed in uncultured CB HSPCs (CD34+CD38−CD90+) selected based on HLF and MYCT1 expression. (c) Dot plot depicting gene expression of selected human HSC signature genes^{17,31-34}, that are significantly differentially expressed in HLF+ HSCs from MYCT1 KD and/or MYCT1 OE compared to control. Uncultured and cultured HLF+ HSCs are also shown. (d) Single cell RNAseq dot plots depicting the scores for the dysregulated functional categories comparing HLF+ HSCs from cultured control, MYCT1 KD and MYCT1 OE HSPC. Uncultured and cultured HLF+ HSCs are also shown. (e) Dot plot depicting gene expression of selected mitochondrial and oxidative phosphorylation (OXPHOS) genes. (f,g) Quantification and representative FACS plots of mitochondrial membrane potential (TMRE) (f) and mitochondrial reactive oxygen species (MitoSOX) (g) in control, MYCT1 KD and MYCT1 OE HSPCs 72 h after transduction. The TMRE and MitoSOX signals are calculated as delta median fluorescence intensity (deltaMFI) between the GFP positive and the GFP negative cells within the same sample. n=3 experiments, mean±s.e.m., two-tailed unpaired t-test. P values for TMRE (f) KD: 0.002, OE: 0.0026, KD vs OE: 0.0002. P values for MitoSOX (g) KD: 0.0091, OE: 0.0034, KD vs OE: 0.0004.

FIG. 23A-I. Restoring MYCT1 expression in cultured human HSPCs improves self-renewal and engraftment ability. (a) Experimental outline for evaluating the effects of rescuing MYCT1 expression in culture on CB HSPC ex vivo expansion and engraftment ability before and after culture expansion. (b) Fold expansion of total live cells, total CD34+CD38− cells, total HSPCs (CD34+CD38−CD90+CD45RA−), and immunophenotypic LT-HSC (CD34+CD38−CD90+CD45RA−EPCR+ITGA3+) from CB transduced with control or MYCT1 OE lentiviral vectors. n=3 experiments, mean±s.e.m, ratio-paired two-tailed t-test. P values from left to right for CD34+CD38−: 0.0221, 0.0255; for total HSPC: 0.0125, 0.0068, 0.0273; for LT-HSC: 0.0180, 0.0176, 0.0071. (c-f) 500 or 2500 HSPC transduced with control or MYCT1 OE vectors were sorted (CD34+CD38−CD90+GFP+) and transplanted 96 hours after transduction into immunodeficient NBSGW mice. c Percentage of mice with human hematopoietic engraftment (≥0.1% hCD45+), multilineage (myeloid, B-lymphoid, and T-lymphoid/other) engraftment, or detectable human erythroid engraftment (hCD71+hGlyA+) in the bone marrow 12 weeks after transplantation. Two-tailed paired t-test. P values from left to right: 0.0024, <0.0001, 0.0056. (d,e) Percentage of total human CD45 cells (d) or undifferentiated human hCD45+CD34+CD38− cells (e) in BM from NBSGW mice 12 weeks after transplantation. Median and all individual values are shown, two-tailed Mann-Whitney test. P values from left to right: 0.0430, ns, 0.0100. (f) Estimation of RU (repopulating unit) frequency within the population of transplanted cells. (g-i) The progeny of 50, 250, 500, or 2500 HSPC transduced with control of MYCT1 OE and sorted for GFP+ were transplanted into immunodeficient NBSGW mice after an additional 10 days in culture (15 days total). (g) Graphs show percentage of mice with human hematopoietic engraftment, multilineage engraftment, or detectable human erythroid engraftment in the bone marrow 12 weeks after transplantation. two-tailed paired t-test. *P value=0.0130. (h) Percentage of human CD45 cells in BM from NBSGW mice 12 weeks after transplantation for all the different doses. Median and all individual values are shown. (i) Estimation of RU frequency within the population of transplanted cells. In f and i the frequency of reconstituting units and P value were calculated using ELDA software⁴¹. (c-f) Transplantations of HSPCs sorted 96 hours after transduction are a total of n=3 donors divided in n=5 transplantations with a total of n=30 mice for each control and MYCT1 OE. n=19 (transplanted with 500 cells) male and female NBSGW mice or n=11 (2500 cells) female NBSGW were transplanted for each control and MYCT1 OE. (g-i) Transplantations of HSPC progeny after 15 days in culture are a total of n=2 donor pools divided in n=4 transplantations, n=1 per dose, with a total of n=28 NBSGW mice for each control and MYCT1 OE. n=5 (transplanted with 50 cells for control), n=6 (50 cells, MYCT1 OE), n=6 (500 cells, control), and n=6 (500 cells, MYCT1 OE) female NBSGW mice, and n=9 (250 cells, control), n=9 (250 cells, MYCT1 OE), n=8 (2500 cells, control), and n=7 (2500 cells, MYCT1 OE) male NBSGW mice were transplanted.

FIG. 24A-H. MYCT1 is located in endosomes and interacts with the vesicle trafficking and receptor signaling machinery. (a-f) Determining the localization of MYCT1 in hematopoietic cells. (a) Transmembrane topology of MYCT1 was predicted from its aminoacid sequence using Phobius⁴⁵ and is depicted as a schematic and histogram of probability. (b) MYCT1 subcellular localization in KG1 hematopoietic cells overexpressing V5-tagged MYCT1 protein, quantified by western blot after cell fractionation. Representative image and relative quantification from n=3 experiments. (c,d) Immunofluorescence localizing overexpressed MYCT1-V5 protein in KG1 cells (c) or human CB HSPCs after 4 day culture (d). MYCT1-V5 was visualized by staining for V5, and co-localization with endosomal markers (Clathrin, Rab5, Rab7, Rab11) and mitochondrial marker HSP60 was evaluated. DAPI indicates nuclei. Colocalization channel (coloc) shows areas positive for V5 and each marker. Scale bars=10 μm. Representative images of n=3 independent experiments for KG1 and n=2 for CB HSPC. (e) Quantification of percentage of MYCT1 colocalization with each marker for KG1 and HSPC. Colocalization analysis was performed with Imaris v9.7.2 software on 9-17.

FIG. 25A-J. MYCT1 controls endocytosis and environmental sensing in human HSPCs and endothelial cells. (a-d) Evaluating the effects of MYCT1 KD and OE on endocytosis using internalization of fluorescent dextran. (a,b) Quantification and representative FACS histogram of internalized fluorescent dextran in CB HSPCs (a) or E4ECs (b) 72 hours after transduction with control, MYCT1 KD or MYCT1 OE lentiviral vectors. n=3 experiments, mean±s.e.m, two-tailed t-test. P values for HSPC KD: 0.0343, OE: 0.0302, KD vs OE: 0.018; for E4EC KD1: 0.0199, KD2: 0.0314. (c) Quantification of internalized fluorescent dextran in different CB HSPC/HPC subsets at different time points in culture. n=4 experiments, mean±s.e.m, two-tailed Mann-Whitney test. P values from left to right: 0.0095, 0.0238, 0.0411. (d) Quantification and representative histogram of internalized fluorescent dextran in CB HSPCs (CD34+CD90+) transduced with control or MYCT1 OE vectors and cultured for 10-12 days. n=4 experiments, mean±s.e.m, two-tailed t-test. P value: 0.0148. (e-g) Evaluating the effects of MYCT1 KD on phosphoproteome in E4EC. Protein centric (e) and phospho-site centric (f,g) analysis of differentially phosphorylated proteins from phospho-mass spectrometry in E4EC 72 hours after transduction with control or two different MYCT1 shRNAs (KD1 and KD2). n=1 experiment in technical duplicates. (e) −Log P values for selected KEGG, Reactome (REAC) and WikiPathway (WP) terms for the proteins with increased (red) or decreased (blue) phosphorylation after MYCT1 KD compared to control. (f) Predicted relative kinase activity (Kinase-Substrate Enrichment Analysis, KSEA⁵¹) and (g) enriched pathways and perturbations (post-translational modification enrichment analysis, PTM-SEA⁵²). (h,i) Evaluating the effects of MYCT1 KD and OE on signaling in HSPCs. Western blot of phospho-AKT in CB HSPCs 72 hours after transduction with control, MYCT1 KD or MYCT1 OE vectors (h), and the respective quantification, relative to control (i). n=3 experiments, mean±s.e.m, two-tailed paired t-test. P values for KD1: 0.0484, for KD2: 0.0014. (j) Single cell RNAseq dot plots for signaling module scores in HSC (HLF+) 72 hours after transduction with control, MYCT1 KD or MYCT1 OE vectors, as well as uncultured and day 4 cultured CB HSC. For (a) and (d), the dextran signal is calculated as delta median fluorescence intensity (deltaMFI) between the GFP positive and the GFP negative cells within the same sample.

FIG. 26A-E. Silencing of MYCT1 expression in cultured human HSPCs. (a-e) Relative expression of MYCT1 and other HSC regulatory genes in sorted FL or CB HSPCs that were isolated freshly and after culture in different conditions. (a) Microarray analysis of CD34+CD38−CD90+ FL HSPCs co-cultured with OP9 stroma supplemented with cytokines as indicated⁸. (b) RNAseq analysis of CD34+CD38−CD90+CD45RA−CD49f+ CB LT-HSPC cultured without stroma and supplemented with LDL and cytokines as indicated²⁷. (c) RT-qPCR of CD34+CD38−CD90+ CB HSPCs co-cultured with E4EC¹⁰ supplemented with LDL, UM171, SR1 and cytokines as indicated. n=2 experiments, mean±s.e.m. (d,e) Gene expression (RNAseq) and UCSC genome browser tracks of MYCT1 and MECOM genomic regions showing RNA seq and Chip-seq of histone marks in CD34+CD38−CD90+ FL HSPCs that were isolated freshly or co-cultured with OP9 stroma supplemented with LDL, UM171, SR1 and cytokines, as indicated¹⁵.

FIG. 27A-C Validation of MYCT1 knockdown and its effects in cord blood HSPCs. (a,b) Validating MYCT1 knockdown at mRNA and protein level. (a) qPCR evaluating relative MYCT1 expression in sorted CB HSPCs (CD34+CD38−CD90+) 72 hours after transduction with control or MYCT1 knockdown (KD) lentiviral vectors. n=3 (KD1) or n=2 (KD2) experiments, mean±s.e.m, two-tailed t-test. P values for KD1: 0.0007, for KD2: <0.0001. (b) Western blot evaluating MYCT1 protein in MYCT1-V5 (OE) KG1 cells after transduction with control or MYCT1 KD lentiviral vectors. n=1 experiment. (c) Representative FACS plots, gating strategy, and quantification of immunophenotypical HSPC/HPC fractions from CB HSPCs transduced with control or MYCT1 KD lentiviral vectors, after 15 days in culture. Percentage of cells in each population within the total cells is indicated. Corresponds to quantifications in FIG. 1e. n=4 experiments, mean±s.e.m., two-tailed Mann-Whitney test. P values from left to right for KD1 CD34+CD38−: 0.0039,0.0028; for KD2 CD34+CD38−: 0.0451, 0.0008; for KD1 total HSPC: 0.0387, 0.0089; for KD2 total HSPC: 0.0186; for KD2 LT-HSC: 0.042, 0.0163.

FIG. 28A-B. Effects of MYCT1 knockdown on HSPC proliferation. (a-b) Quantifying proliferation in HSPCs after MYCT1 KD. (a) Percentage of cells that have undergone at least 1 to 4 divisions, or no divisions, for every 24 hours elapsed since plating. HSPCs (CD34+CD38−CD90+) were sorted 72-96 hours after transduction (day 4 in culture), plated as single cells in 96-well plates, and monitored individually every 24 hours. n=4 experiments, mean±s.e.m, two-tailed t-test. P values at the 96 hours timepoint, from left to right for KD1: 0.0094, 0.0025, 0.0243, 0.0094, 0.0027; for KD2: 0.0001, 0.0002, 0.004, 0.0001, 0.0008. (b) Representative FACS histograms and quantification of proliferation by dye dilution (CellTrace) in control or MYCT1 KD CB HSPCs (CD34+CD38−CD90+) at the time of labelling (72-96 hours after transduction, indicated as 0 h) and after an additional 48 hours. n=4 (KD1) and n=3 (KD2) experiments, mean±s.e.m, two-tailed Mann-Whitney t-test. For KD1 *P=0.0317, for KD2 *P=0.0159.

FIG. 29A-D. Documentation of the effects of MYCT1 knockdown on human foetal liver HSPC expansion and engraftment. (a,b) Quantifying expansion of foetal liver (FL) HSPC after MYCT1 KD. Fold expansion (a), percentage and representative FACS plots (b) of all live cells and immunophenotypical self-renewing HSCs (CD34+CD38−CD90+GPI80+) from control or MYCT1 KD FL. n=2 experiments, mean±s.e.m. (c,d) Determining the transplantation ability of MYCT1 KD FL HSPCs. (c) FACS plots showing control or MYCT1 KD CB HSPCs sorted for transplantation 72 hours after transduction. The black gate indicates the population of FL HSPCs (CD34+CD38−CD90+GPI80+) that were sorted and transplanted. 10,000 sorted control or KD HSPCs were transplanted per mouse. (d) Representative FACS plots and quantification of human hematopoietic engraftment (human CD45+) and multilineage differentiation in NSG mice 24 weeks after transplantation. In FACS plots, differentiated cells are quantified as percentage of total hCD45+ cells. Quantification shows bone marrow at 6, 12 and 24 weeks after transplantation, and spleen and blood 24 weeks after transplantation. n=5 (control) and n=4 (MYCT1 KD) mice per group, mean±s.e.m., two-tailed Mann-Whitney test. *P values from left to right: 0.0238, 0.0357, 0.0357, 0.0357.

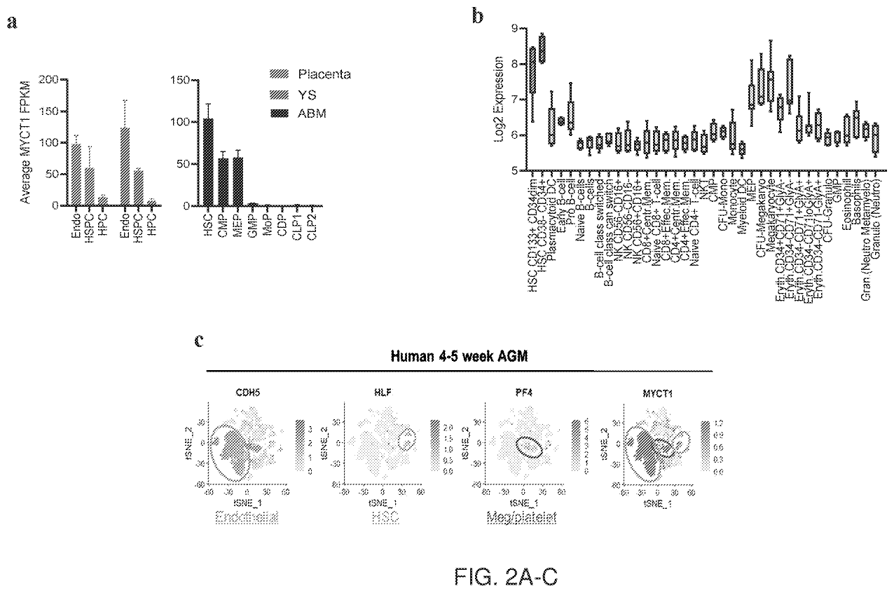

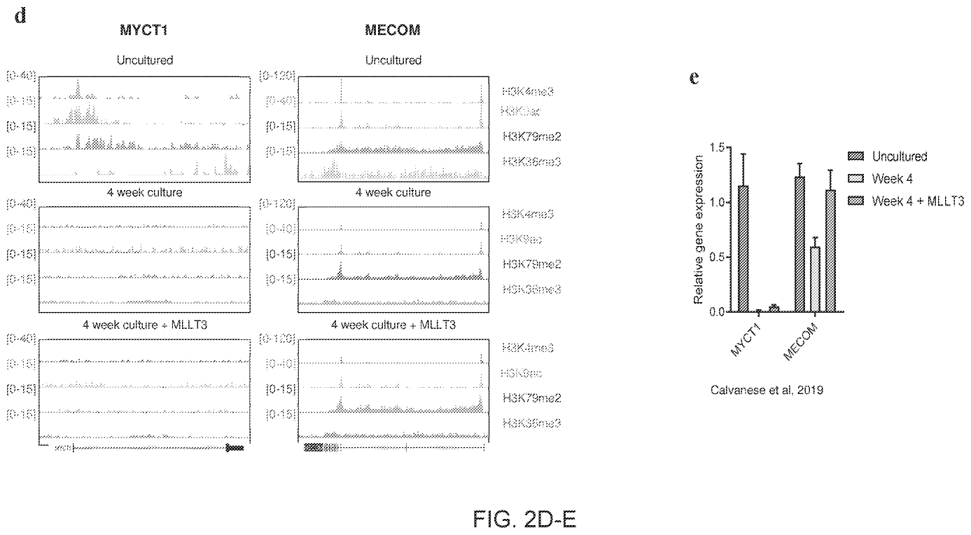

FIG. 30A-J. Single cell analysis of MYCT1 dependent programs in cultured human HSCs. (a,b) Number of total HSPCs and HLF+ cells sequenced for scRNAseq for each sample (a) and percentage of HLF+ cells within each sequenced sample (b). c,d TSNE plots showing all the sequenced HSPCs (d), or the selected HLF+ cells 72 hours after transduction (e). (e-j) scRNAseq dot plots depicting expression for genes in the programs found to be differentially regulated by MYCT1 KD or OE in HLF+ HSC (see FIG. 2d). n=1 experiment with the indicated number of single cells sequenced.

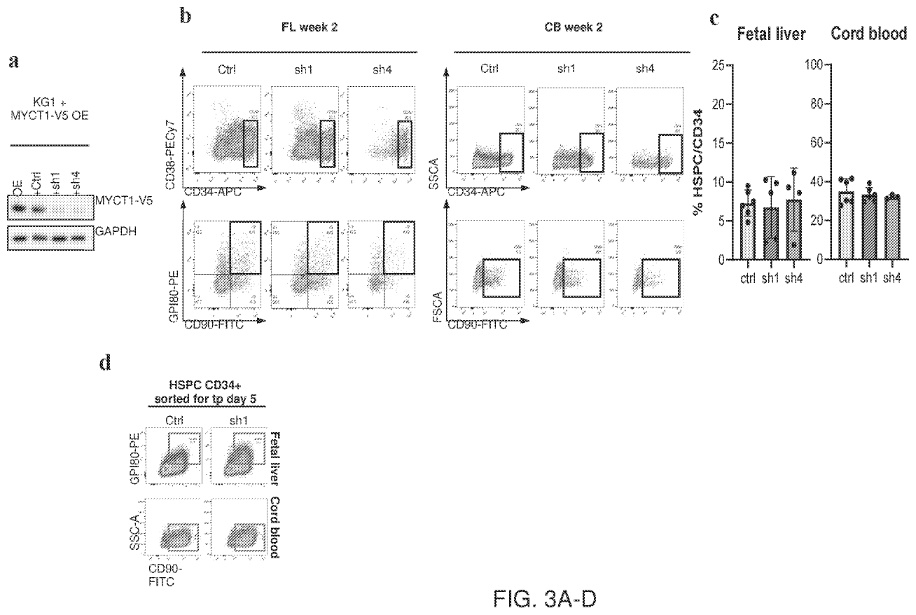

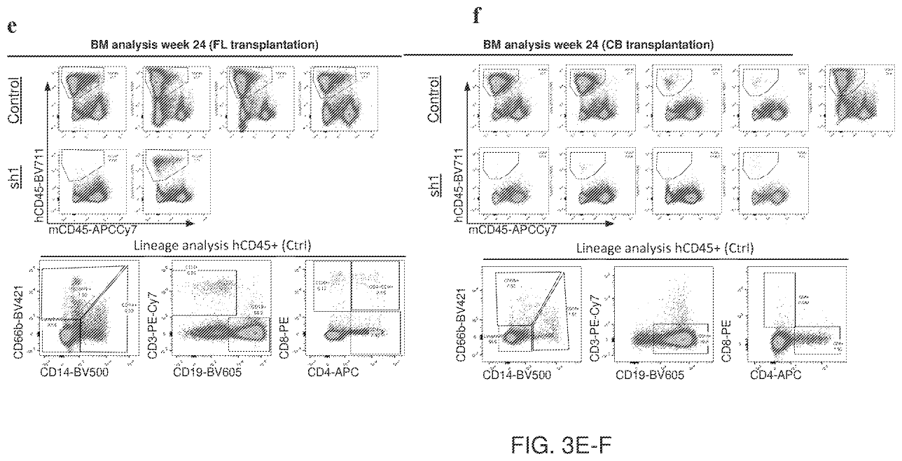

FIG. 31A-E. Effects of restoring MYCT1 expression on human HSPCs in culture. (a) qPCR evaluating relative MYCT1 expression in sorted CB HSPCs 72 hours after transduction with control or MYCT1 OE lentiviral vectors (CD34+CD38−CD90+GFP+). n=3 experiments, mean±s.e.m. (b) Representative FACS plots and gating strategy (b), and percentage (c) of undifferentiated CD34+CD38− cells, HSPCs (CD34+CD38−CD90+CD45RA−), and immunophenotypic LT-HSC (CD34+CD38−CD90+CD45RA−EPCR+ITGA3+) expanded from control or MYCT1 OE CB HSPCs after 10, 15 and 20 days in culture. Corresponds to quantifications in FIG. 3b. Percentage of cells in each population within the total cells is indicated in the FACS plots. n=3 experiments, mean±s.e.m, ratio-paired two-tailed t test. P values from left to right for CD34+CD38−: 0.004, 0.0031, 0.0026; for total HSPC: 0.0159, 0.0018, 0.0233; for LT-HSC: 0.0197, 0.0049, 0.0069. (d) Number and type of colonies formed in enriched methylcellulose (see Methods) after plating equal number of CB HSPCs transduced with control or MYCT1 OE and sorted (CD34+CD38−CD90+GFP+) 72-96 hours after transduction. CFU/BFU-E: Colony-forming unit-erythroid or burst-forming unit-erythroid erythroid, CFU-GM: granulocyte and/or macrophage, CFU-Mixed: granulocyte, erythroid, macrophage. n=4 experiments, mean±s.e.m, paired two-tailed t test. P values: 0.00177 for total colonies, 0.0446 for CFU-Mixed. (e) Percentage of cells that undergo 1 to 4 divisions, or no divisions observed for every 24 hours elapsed since plating. Determined by individual monitoring every 24 hours of single sorted control and MYCT1 OE HSPCs starting 72 hours after transduction (day 4 in culture). n=3 experiments, mean±s.e.m.

FIG. 32A-D. Multilineage differentiation after transplantation of MYCT1 overexpressing HSPCs. (a) Table indicating the number of mice transplanted with control or MYCT1 OE HSPC for each condition per replicate, for the transplantations performed 96 hours after transduction. (b) Gating strategy and representative FACS plots showing human myeloid (CD14+ or CD66b+), B-lymphoid (CD19+), T-lymphoid/other (CD3, CD4 or CD8+), erythroid (GlyA+CD71+), and undifferentiated (CD34+CD38−) populations of mice transplanted with 500 or 2500 HSPCs 96 hours after transduction. (c) Table indicating the number of mice transplanted with the progeny of control or MYCT1 OE HSPC after 15 days in culture for each condition and cord blood sample. (d) Representative FACS plots showing the human differentiated populations as above of mice transplanted with the progeny of 50, 250, 500 or 2500 HSPCs after 15 days in culture. (a,b) correspond to the experiments shown in FIG. 23c-f, (c,e) correspond to the experiments shown in FIG. 23g-i.

FIG. 33A-K. Restoration of MYCT1 associated programs in Soluplus expanded HSCs. (a-c) Quantifying the maintenance of MYCT1+ HSC in standard culture conditions supplemented with cytokines and small molecules (SR1 or UM171), or the novel Soluplus-based HSC expansion culture from the Sakurai et al scRNAseq dataset¹⁴. (a) Percentage of HLF+ HSCs cells among the total CD34+ cells in day 10 expansion culture. (b) Percentage of MYCT1+ cells within the expanded HLF+ HSC from the Sakurai dataset¹⁴, compared to uncultured CB HSC (from FIG. 2). (c) scRNAseq dot plot depicting MYCT1 expression in HLF+ HSC after 10 days in culture with different conditions. (d-k) Evaluating MYCT1 associated programs in the standard culture conditions and the novel human HSC expansion culture from Sakurai et al scRNAseq dataset¹⁴. Single cell RNAseq dot plots depicting module scores (d) and gene expression (e-k) for the programs that were found to be governed by MYCT1. HLF+ HSC cultured for 10 days in SR1, UM171 or Soluplus-based conditions¹⁴ (green box) are compared to control, MYCT1 KD and MYCT1 OE HLF+ (grey box) (from FIG. 22 and Extended Data FIG. 27).

FIG. 34A-G. Dissecting the MYCT1 interactome in KG1 hematopoietic cells and endothelial cells. (a-c) Validating KG1 and E4EC as suitable models for studying MYCT1 molecular function in HSC. (a) FACS analysis of human HSC surface markers CD34 and CD90 in KG1 and human CB HSPCs. n=1 experiment. (b) Gene expression (RNAseq) for selected HSC genes in KG1 and K562 cell lines from the Cancer Cell Line Encyclopaedia⁷⁷. (c) Fold expansion of E4EC and HUVEC cells 15 days after transduction with control or MYCT1 KD vectors. n=4 (E4EC) or n=3 (HUVEC) experiments, mean±s.e.m., two-tailed ratio paired t-test. P values from left to right for KD1: 0.0175, 0.00053; for KD2: 0.0006, 0.0071. (d-g) Dissecting the MYCT1 interactome. (d) Western blot for immunoprecipitation using anti-V5 antibody in KG1 transduced with control or overexpression of V5-tagged MYCT1. (e) Identification of MYCT1 interacting proteins in KG1 cells and E4EC using high-sensitivity mass spectrometry. Heatmap of all the specific MYCT1 interactors detected by immunoprecipitation of V5 tag in control or MYCT1-V5 overexpressing KG1 and E4EC cells. Scale depicts the number of detected peptides for each protein. Colours indicate protein categories. All technical duplicates from n=3 (KG1) or n=1 (E4EC) experiments are shown. (f) Heatmaps of −Log P values of GO, Reactome (REAC), KEGG, and WikiPathway (WP) terms for the MYCT1 interactome in KG1 cells and E4EC. (g) Heatmaps depicting the MYCT1 interactors detected in KG1 and E4EC by number of peptides, and their average expression from RNAseq in KG1, E4EC (in RPKM) and sorted ECs and HSPCs from embryonic tissues (5-6 week AGM, YS=yolk sac, PL=placenta), second trimester FL, CB and adult bone marrow (ABM) (in FPKM).

FIG. 35A-K. Evaluation of MYCT1 moderated signaling responses. (a) Experimental outline for phospho-proteomic profiling and analysis in E4EC 72 hours after transduction with control or MYCT1 KD vectors. (b) Volcano plots depicting Log 2 fold change and −Log 10 P value for all identified phosphorylated protein sites in E4EC transduced with control or two different KD vectors. (c) Heatmap depicting the Log 2FC in the same samples as (b) to compare the effect of both KDs. The Pearson correlation between the two different MYCT1 KD vectors calculated using Excel is included. n=1 experiment in technical duplicates. (e-k) Determining the effect of MYCT1 KD on the signaling responses to cytokines by western blot. e Experimental outline for cytokine stimulation time course experiments in control or MYCT1 KD E4EC 72 hours after transduction. (f-h) Representative western blot (f) and quantification of phospho-AKT and phospho-ERK in control and MYCT1 KD E4ECs after overnight starvation and subsequent stimulation with complete media (containing serum, FGF, EGF and IGF) for 30 minutes, 1 and 3 hours (g). The quantifications under starvation conditions are also shown zoomed in (h). n=4 (KD1) or n=5 (KD2) experiments, mean±s.e.m, two-tailed ratio-paired t-test. P values in g from left to right for KD1: 0.0152 (pAKT), 0.0491, 0.0288 (pERK1); for KD2: 0.0211, 0.0359 (pAKT), 0.0442, 0.0045, 0.00515 (pERK1), 0.0095, 0.0267 (pERK2). P values in h from left to right for KD1: 0.009 (pERK1), 0.0305 (PERK2); for KD2: 0.0281 (pAKT), 0.0031 (PERK1), 0.0028 (PERK2). (i) Experimental outline to quantify the basal signaling responses in control and MYCT1 KD E4EC cultured with continuous presence of cytokines. j,k Representative western blot (j) and quantification (k) of phospho-AKT and phospho-ERK in control and MYCT1 KD E4ECs under regular culture conditions with complete media. n=3 experiments, mean±s.e.m, two-tailed ratio-paired t-test. P values from left to right for KD1: 0.0093 (pERK1), 0.0132 (pERK2); for KD2: 0.0080 (pAKT), 0.0067 (pERK1), 0.0218 (pERK2).

### DETAILED DESCRIPTION

As disclosed herein in certain aspects, MYCT1-moderated environmental sensing through the control of endocytosis is an essential mechanism required to preserve human HSC stemness and pinpoints MYCT1 downregulation as a critical contributor to the dysfunction of cultured HSCs. Accordingly, aspects of the present disclosure include methods and compositions for improving HSC health and function comprising increasing expression and/or activity of MYCT1 in cultured HSCs. Such improved HSCs may provide better in vitro models for PSC-derived hematopoiesis and allow for enhanced engraftment and viability of transplanted HSCs for treatment of various conditions.

Hematopoietic stem cells (HSC) rely on a unique regulatory machinery to facilitate lifelong blood production and reconstitute the hematopoietic system upon transplantation. However, the processes governing human HSC self-renewal and engraftment ability are poorly understood and challenging to recapitulate ex vivo to achieve robust expansion of functional HSCs. Aspects herein show a novel HSC regulatory protein, MYCT1 (MYCT target 1), that is selectively expressed in endothelial cells (EC) and undifferentiated human HSPCs but becomes drastically downregulated during HSC culture. Lentiviral knockdown of MYCT1 in human foetal liver and cord blood HSPCs uncovered a critical function for MYCT1 in human HSPC expansion and engraftment. Single cell RNAseq of MYCT1 knockdown and overexpressing human CB HSPCs revealed that MYCT1 governs critical HSC regulatory programs and maintains cellular properties essential for HSC stemness, such as low mitochondrial metabolic activity. Restoring the compromised MYCT1 expression in cultured human CB HSPCs improved expansion of undifferentiated human HSPCs and enhanced their engraftment ability. Aspects herein show MYCT1 is localized in the endosomal membrane where it interacts with vesicle trafficking regulators and signaling machinery essential for HSC and EC function. Aspects herein show MYCT1 loss leads to excessive endocytosis and hyperactive signaling responses to cytokines, whereas restoring MYCT1 expression in cultured CB HSPCs balanced the abnormal endocytosis associated with prolonged culture and fine-tuned signaling responses. Aspects herein identify MYCT1-moderated endocytosis and environmental sensing as an essential regulatory mechanism required to preserve human HSC stemness, and pinpoints silencing of MYCT1 as a critical contributor to the dysfunction of cultured human HSCs that needs to be addressed to optimize human HSC ex vivo expansion.

### I. Administration of Therapeutic Compositions

Aspects of the disclosure relate to compositions and methods comprising therapeutic compositions, which can include nucleic acids (such as those capable of introducing MYCT1 to a cell), gene editing systems (such as those capable of introducing MYCT1 to a cell), and/or cellular therapies (such as modified hematopoietic stem cell having increased expression or activity of MYCT1). The different therapies may be administered in one composition or in more than one composition, such as 2 compositions, 3 compositions, or 4 compositions. Various combinations of the agents may be employed.

The therapeutic compositions of the disclosure may be administered by the same route of administration or by different routes of administration. In some aspects, the therapeutic composition is administered intratumorally, intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, a single dose of the therapeutic composition is administered. In some aspects, multiple doses of the therapeutic composition are administered. In some aspects, the therapeutic composition is administered at a dose of between 1 mg/kg and 5000 mg/kg. In some aspects, the BAMBI composition is administered at a dose of between 1 mg/kg and 5000 mg/kg. In some aspects, the therapeutic composition is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

In some aspects, the therapeutic composition is administered at a dose of between 1×10³ to 9×10¹² cells (or any range derivable therein), including when the therapeutic composition comprises cells disclosed herein, such as a population of immune cells disclosed herein. In some aspects, the therapeutic composition is administered at a dose of approximately 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹² cells (or any range derivable therein).

### A. Nucleic Acids

In certain embodiments, there are recombinant nucleic acids encoding the proteins, polypeptides, or peptides described herein. Polynucleotides contemplated for use in methods and compositions include those encoding MYCT1. Also contemplated are polynucleotides encoding MLLT3.

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

The quantity to be administered, both according to number of treatments and unit dose, depends on the treatment effect desired. The term “therapeutic benefit” or “therapeutically effective” as used throughout this application refers to anything that promotes or enhances the well-being of the subject with respect to the medical treatment of cancer. This includes, but is not limited to, a reduction in the frequency or severity of the signs or symptoms of a disease. For example, treatment of cancer may include but is not limited to total or partial remission of the cancer. Treatment of cancer may also refer to prolonging survival of a subject with a cancer. The term “therapeutically effective amount” refers to an amount sufficient to produce a desired therapeutic result, for example an amount of a BAMBI composition and/or a cancer therapy or a composition comprising such a BAMBI composition and/or a cancer therapy sufficient to improve at least one symptom of a medical condition in a subject to whom the BAMBI composition and/or a cancer therapy or composition thereof are administered.

In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### B. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 1. Host Cells

As used herein, the terms “cell” and “cell culture” may, in some cases, be used interchangeably. All of these terms also include their progeny, which is any and all subsequent generations. It is understood that all progeny may not be identical due to deliberate or inadvertent mutations. In the context of expressing a heterologous nucleic acid sequence, “host cell” or target cell includes any transducable or otherwise engineerable organism that is capable of replicating a vector or plasmid, expressing a heterologous gene encoded by a vector or plasmid, and/or otherwise expressing an exogenous nucleic acid molecule. A host or target cell may, in certain cases, be “transfected” or “transduced,” which refers to a process by which exogenous nucleic acid, such as a recombinant protein-encoding sequence, is transferred or introduced into the host or target cell. A transduced or otherwise engineered cell includes the primary subject cell and its progeny. In some aspects, a target cell is a hematopoietic cell or endothelial cell. In some aspects, the target cell is a hematopoietic stem cell.

In another aspect, contemplated are the use of host cells into which a recombinant expression vector has been introduced. An expression construct can be transfected into cells according to a variety of methods known in the art. Vector DNA can be introduced into cells via conventional transduction or transfection techniques. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics or expression of a fluorescent protein) is generally introduced into the host cells along with the gene of interest. Cells stably transfected with the introduced nucleic acid can be identified by drug selection (e.g., cells that have incorporated the selectable marker gene will survive, while the other cells die) or expression of a fluorescent protein, among other methods known in the arts.

In some aspects, host cells are transiently transfected with a nucleic acid molecule encoding a polypeptide of interest. For example, in certain cases, a host cell is transfected with an mRNA encoding a polypeptide (e.g., MYCT1 and/or MLLT3).

### 2. Vectors

Polypeptides may be encoded by a nucleic acid molecule. The nucleic acid molecule can be in the form of a nucleic acid vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Expression vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). Vectors may be used to target hematopoietic cells and hematopoietic stem cells to allow the maintenance and expansion of HSCs, HSPCs, iPSC-HSPCs and hESC-HSPCs in culture.

In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

a. Viral Vectors

### (1) Adenoviral Infection

One method for delivery of the recombinant DNA involves the use of an adenovirus expression vector. Although adenovirus vectors arc known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a recombinant gene construct that has been cloned therein.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the some starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells.

### (2) Retroviral Infection

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

In some aspects, a retroviral vector of the present disclosure is a lentiviral vector.

### (3) AAV Infection

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin et al., 1984; Laughlin et al., 1986; Lebkowski et al., 1988; Mclaughlin et al., 1988), which means it is applicable for use with the present methods and compositions. Details concerning the generation and use of rAAV vectors are described in U.S. Pat. Nos. 5,139,941 and 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; Mclaughlin et al., 1988) and genes involved in human diseases (Flotte et al., 1992; Ohi et al., 1990; Walsh et al., 1994; Wei et al., 1994). Recently, an AAV vector has been approved for phase I human trials for the treatment of cystic fibrosis.

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994a; Clark et al., 1995). Cell lines carrying the rAAV DNA as an integrated provirus can also be used (Flotte et al., 1995).

### B. Non-Viral Delivery

In addition to viral delivery of the expression vectors encoding transcription factors, the following are additional methods of recombinant gene delivery to a given host cell and are thus considered in the present invention.

### (1) Lipid Mediated Transfection

In a further embodiment of the invention, an expression vector may be entrapped in a liposome or lipid formulation. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Advances in lipid formulations have improved the efficiency of gene transfer in vivo (Smyth-Templeton et al., 1997; WO 98/07408). A novel lipid formulation composed of an equimolar ratio of 1,2-bis(oleoyloxy)-3-(trimethyl ammonio)propane (DOTAP) and cholesterol significantly enhances systemic in vivo gene transfer, approximately 150-fold. The DOTAP:cholesterol lipid formulation is said to form a unique structure termed a “sandwich liposome”. This formulation is reported to “sandwich” DNA between an invaginated bi-layer or ‘vase’ structure. Beneficial characteristics of these lipid structures include a positive colloidal stabilization by cholesterol, two dimensional DNA packing and increased serum stability.

In further embodiments, the liposome is further defined as a nanoparticle. A “nanoparticle” is defined herein to refer to a submicron particle. The submicron particle can be of any size. For example, the nanoparticle may have a diameter of from about 0.1, 1, 10, 100, 300, 500, 700, 1000 nanometers or greater. The nanoparticles that are administered to a subject may be of more than one size.

Any method known to those of ordinary skill in the art can be used to produce nanoparticles. In some embodiments, the nanoparticles are extruded during the production process. Exemplary information pertaining to the production of nanoparticles can be found in U.S. Patent App. Pub. No. 20050143336, U.S. Patent App. Pub. No. 20030223938, and U.S. Patent App. Pub. No. 20030147966, each of which is herein specifically incorporated by reference into this section.

One of ordinary skill in the art would be familiar with use of liposomes or lipid formulation to entrap nucleic acid sequences. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

In some embodiments, instead of introducing MYCT1 into a hematopoietic stem cell, cells are exposed to or incubated with an endocytosis inhibitor. Any combination of these (or other known) endocytosis inhibitors may be used. Moreover, it is contemplated in some embodiments that MYCT1 may be introduced into a cell that is or was also exposed to an endocytosis inhibitor. In some embodiments, one or more of these (or other known) endocytosis inhibitors may be excluded. Various endocytosis inhibitors are recognized in the art and contemplated herein. Example endocytosis inhibitors which may be used in methods and compositions of the present disclosure include, but are not limited to, Chlorpromazine, Methyl-β-cyclodextrin, and dynamin inhibitors (e.g., Dynasore, Dyngo®-4A). In some aspects, an endocytosis inhibitor of the present disclosure may specifically inhibit clathrin-mediated endocytosis.

### 3. Expression Systems

Numerous expression systems exist that comprise at least a part or all of the compositions discussed above. Prokaryote- and/or eukaryote-based systems can be employed for use with an embodiment to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems, other examples of expression systems include STRATAGENE®'s COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### II. Formulations and Culture of Cells

In particular embodiments, the cells (e.g., HSCs, modified HSCs) of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific embodiments, one or more of these may be explicitly excluded.

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21™ supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific embodiments, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific embodiments, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

### III. Gene Editing Systems

Certain aspects of the present disclosure relate to methods and compositions for gene editing (also “genetic engineering”), useful in the generation of one or more genetic modifications in a cell (e.g., genetic modifications which increase MYCT1 expression or activity). As used herein, a “genetic modification,” describes a region of a genome of a cell that has been altered from its native (i.e., endogenous) sequence. A genetic modification may be developed via artificial editing of a gene or other genetic material. In some embodiments, a genetic modification is a mutation of a gene. In some embodiments, a mutation is an insertion, a deletion, a point mutation, a frameshift mutation, or a nonsense mutation. One or more of these mutations may be excluded from embodiments of the disclosure. In some embodiments, a mutation prevents expression of a gene (i.e., is a knockout mutation). In some embodiments, a mutation causes production of a mutant form of a protein. In some embodiments, a genetic modification of the disclosure is a mutation of MYCT1.

Certain embodiments of the disclosure are directed to the use of gene editing techniques to generate a knockout mutation in a gene in a population of cells. The disclosed techniques may eliminate expression of the gene in some or all of the cells in the population. In some embodiments, expression of the gene is not detectable in the population of cells (e.g., measured by mRNA and/or protein expression). In some embodiments, expression of the gene is substantially decreased in the population of cells compared with cells not having the knockout mutation. In some embodiments, expression of the gene (e.g., measured by mRNA and/or protein expression) is decreased by at least, at most, or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, 99.9%, or any range or value derivable therein. In some embodiments, expression of the gene is decreased by at least 80%, 90%, 95%, or 99%. In some embodiments, expression of the gene is decreased by at least 90%.

Various methods and systems for gene editing are known in the art and include, for example, zinc finger nuclease (ZFN)-based gene editing, transcription activator-like effector nuclease (TALEN)-based gene editing, and CRISPR/Cas-based gene editing. In some embodiments, methods of the present disclosure comprise CRISPR/Cas-based gene editing, which comprises the use of components of a CRISPR system, for example a guide RNA (gRNA) and a Cas nuclease.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. A Cas nuclease and a gRNA can be introduced into the cell indirectly via introduction of one or more nucleic acids (e.g., vectors) encoding for the Cas nuclease and/or the gRNA. A Cas nuclease and a gRNA can be introduced into the cell directly by introduction of a Cas nuclease protein and a gRNA molecule. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA may be targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% sequence complementarity along the length of the tracr mate sequence when optimally aligned.

One or more vectors driving expression of one or more elements of a CRISPR system can be introduced into a cell such that expression of the elements of the CRISPR system direct formation of a CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding a Cas protein (also “Cas nuclease”). Non-limiting examples of Cas proteins include Cas1, Cas1B, Cas2, Cas3, Cas4, Cas5, Cas6, Cas7, Cas8, Cas9 (also known as Csn1 and Csx12), Cas10, Cas12a (Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1,Csx15,Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The Cas nuclease can be Cas9 (e.g., from S. pyogenes or S. pneumonia). The Cas nuclease can be Cas12a. The Cas nuclease can direct cleavage of one or both strands at the location of a target sequence, such as within the target sequence and/or within the complement of the target sequence. The vector can encode a Cas nuclease that is mutated with respect to a corresponding wild-type enzyme such that the mutated Cas nuclease lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is or is more than 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The Cas nuclease may be part of a fusion protein comprising one or more heterologous protein domains. A Cas nuclease fusion protein may comprise any additional protein sequence, and optionally a linker sequence between any two domains. Examples of protein domains that may be fused to a Cas nuclease, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A Cas nuclease may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a Cas nuclease are described in US 20110059502, incorporated herein by reference.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—The Endosomal Adaptor Protein MYCT1 Controls Environmental Sensing in Human Hematopoietic Stem Cells

Human HSCs sustain life-long blood production and can reconstitute the entire hematopoietic system after transplantation. However, the biological processes governing HSC self-renewal and engraftment ability are still poorly understood, and cannot be recapitulated ex vivo to facilitate robust human HSC expansion. The inventors discovered that MYCT1 (MYCT target 1), a gene selectively expressed in endothelial cells (EC) and undifferentiated human HSPCs, becomes drastically downregulated during HSC culture, concomitantly with the loss of engraftment potential. Knockdown experiments in human foetal liver and cord blood HSPCs revealed that MYCT1 is critical for their expansion and engraftment. Single cell RNAseq in human HSPCs after MYCT1 knockdown and overexpression revealed that MYCT1 governs critical HSC hallmarks and modulates multiple cellular functions essential for HSC stemness. The inventors discovered that MYCT1 is an endosomal membrane protein that interacts with vesicle trafficking regulators and signaling components essential for HSC biology. The inventors further showed that MYCT1 controls endocytosis, and thus the signaling responses from surface receptors. Loss of MYCT1 led to excessive endocytosis and hyperactive signaling responses to cytokines in the culture microenvironment. The inventors also discovered that endocytosis becomes gradually hyperactivated in human HSPCs during ex vivo culture, concomitantly with the silencing of MYCT1 and loss of HSC transplantability. Restoring MYCT1 expression in cultured HSPCs was able to balance the abnormal endocytosis and signaling, and improve engraftment of human HSPCs in immunodeficient mice. The inventors' study identifies MYCT1-moderated environmental sensing through the control of endocytosis as an essential mechanism required to preserve human HSC stemness and pinpoints MYCT1 downregulation as a critical contributor to the dysfunction of cultured HSCs, thereby opening up new avenues to improve human HSC culture strategics.

### MYCT1 Expression is Highly Enriched in Human HSC but Lost in Culture

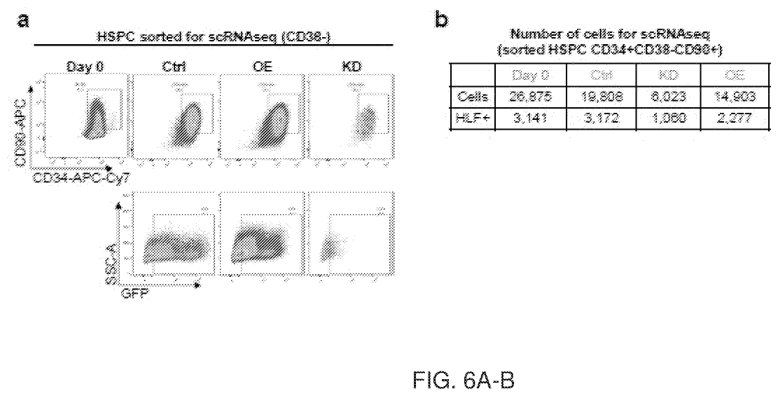

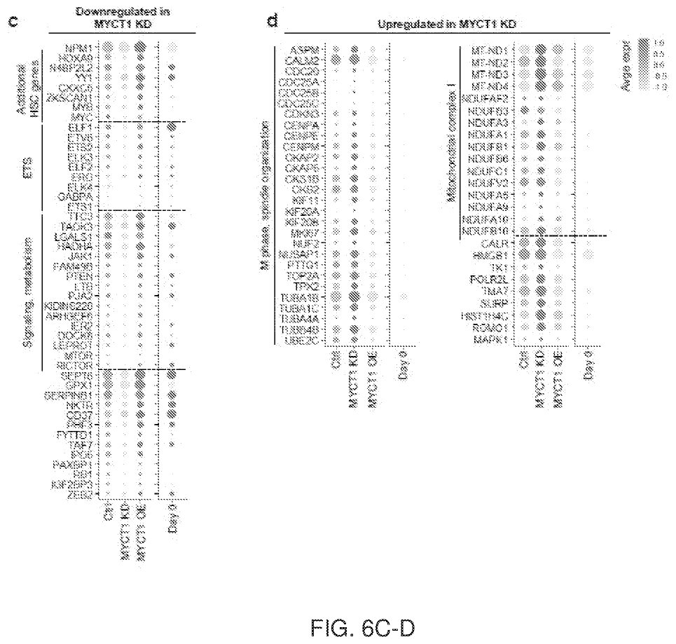

To identify regulators of human HSC self-renewal that become dysregulated in cultured HSCs, the inventors analysed multiple bulk and single cell RNA sequencing (RNAseq) datasets of human developmental and postnatal hematopoietic tissues, and searched for genes that are enriched in undifferentiated human HSPCs but become downregulated during culture. The inventors found that MYCT1 (Myc target 1, also known as MTLC) is selectively expressed in the most undifferentiated HSPC compartment in all tissues and developmental stages (AGM, placenta, yolk sac, fetal liver, cord blood, adult bone marrow), as well as in the megakaryocytic lineage and endothelial cells (ECs) (FIG. 1A, FIGS. 2A-2C). scRNAseq of uncultured CB HSPCs sorted for CD34+CD38−CD90+ (FIGS. 6A-C) revealed that expression of HSC hallmark genes (e.g. MLLT3, AVP) in HSCs identified by HLF expression was further enriched in MYCT1+ vs, MYCT1− HSCs (HLF+) and HPCs (HLF−), while CDK6 expression, which is associated with short-term repopulating HSCs (ST-HSC) and is lower in long-term (LT) HSC, was restricted to MYCT1-HSPC (FIG. 1B). The inventors also found MYCT1 expression to be most highly enriched within the fractions that were most enriched for the transplantable, self-renewing HSCs in FL (GPI80+ fraction) and ex-vivo expanded CB HSPCs (ITGA3+ fraction) (FIGS. 1C and 1D). Importantly, MYCT1 becomes drastically downregulated and epigenetically silenced in cultured human HSPCs (FIGS. 1E and 1F, FIG. 2D), concomitant to the loss of engraftment ability. These data show that MYCT1 is associated with self-renewing human HSCs and suggest that MYCT1 may perform essential functions specific for HSCs.

Furthermore, MYCT1 expression was minimally rescued by overexpression (OE) of MLLT3 (FIG. 2E), a critical regulator of human HSC identity and self-renewal, that maintains the expression of multiple HSC regulatory genes (e.g. MECOM) in cultured HSPCs. This suggests that MYCT1 may be acting independently rather than downstream of MLLT3, and its loss in culture may contribute to the compromised engraftment ability of most of the ex vivo expanded immunophenotypic HSPCs.

### MYCT1 is Functionally Required for Human HSC Expansion and Engraftment

To determine if MYCT1 is required for human HSC function, the inventors performed lentiviral shRNA-mediated knockdown (KD) on sorted human fetal liver (FL) or cord blood (CB) HSPCs and quantified their culture expansion and engraftment ability (FIGS. 1G-1K, FIGS. 3A-3F). MYCT1 KD prevented the expansion of HSPCs (CD34+CD38−CD90+ and GPI80+ in the case of FL), and the generation of differentiated progeny in culture (FIGS. 1I and 1J, FIGS. 3A and 3B). Moreover, MYCT1 KD abrogated HSC engraftment upon transplantation into NSG mice, even when equal number of HSPCs sorted for the same surface phenotype transplanted, while control cells were able to generate multilineage engraftment (FIG. 1K, FIGS. 3D-3F). Similar to human HSPCs, MYCT1 KD also impaired the growth of human endothelial cell line E4EC and primary HUVEC cells (FIG. 1L and FIGS. 3A and 3B). These data identify MYCT1 as a novel human HSC gene that is critical for their expansion and function.

### MYCT1 Governs HSC Hallmarks and Proliferation

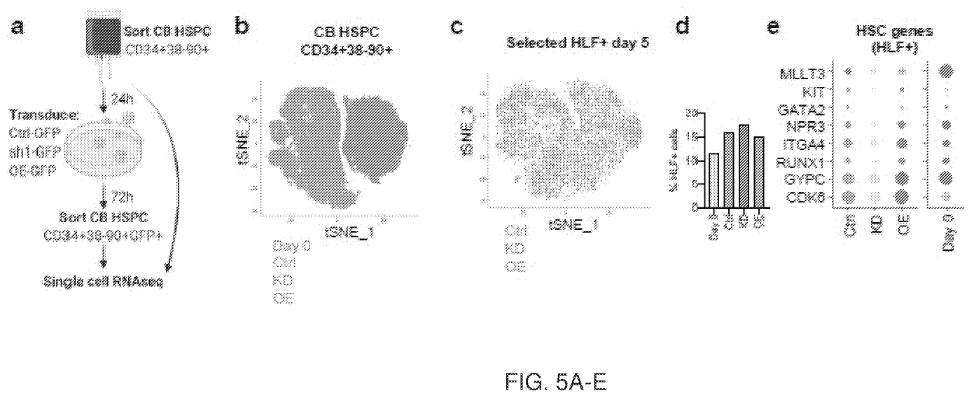

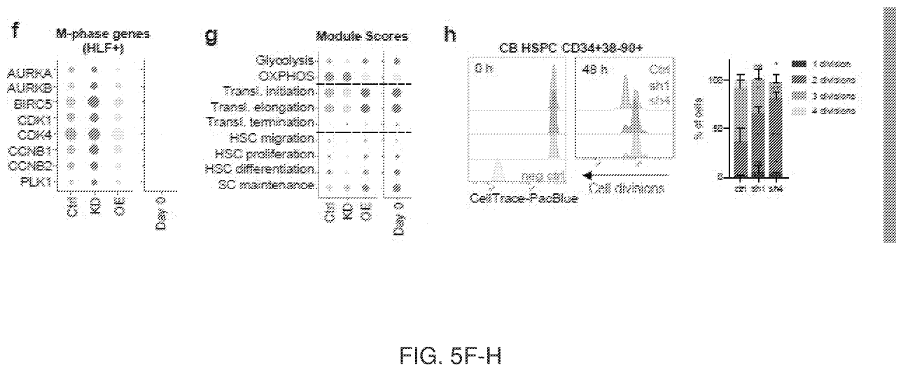

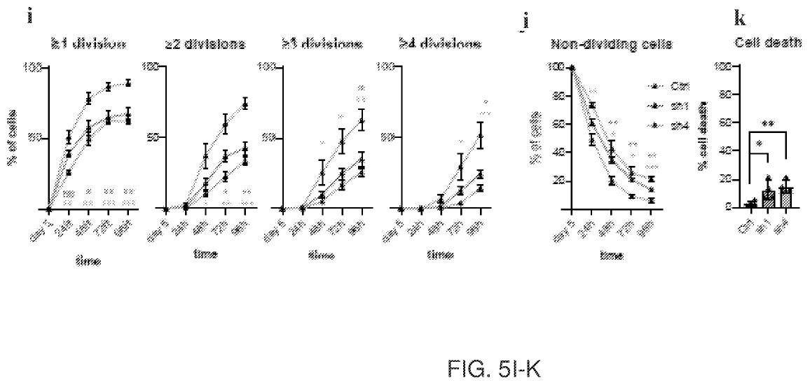

To dissect why MYCT1 is required for human HSC function, the inventors performed scRNAseq in control, MYCT1 KD and OE HSPCs that were sorted (CD34+CD38−CD90+) and sequenced after 5 days in culture in parallel with uncultured HSPCs from the same donor (FIG. 1B, FIGS. 5A and 5B, FIGS. 6A and 6B). Despite being unable to engraft upon transplantation, MYCT1 KD showed a similar proportion of HLF+ cells within the sorted HSPC population as control or MYCT1 OE samples (FIGS. 5C and 5D). However, loss of MYCT1 in HSC led to a profound dysregulation of multiple programs including downregulation of HSC “stemness” genes and ETS transcription factors, and upregulation of M-phase genes and genes involved in the mitochondrial oxidative phosphorylation (Oxphos) system (mitochondrial complex I), among others. Module score analysis revealed that MYCT1 KD HSCs show a metabolic switch form glycolysis to Oxphos, which has been linked to decreased HSC function,. Moreover, MYCT1 KD HSCs showed lower scores for translation, HSC migration, proliferation, differentiation and stem cell maintenance. Importantly, overexpression (OE) of MYCT1 had the opposite effect on those transcriptional signatures and module scores, and showed expression signatures that were more similar to uncultured cells (FIGS. 5E-5G, FIGS. 6C and 6D).

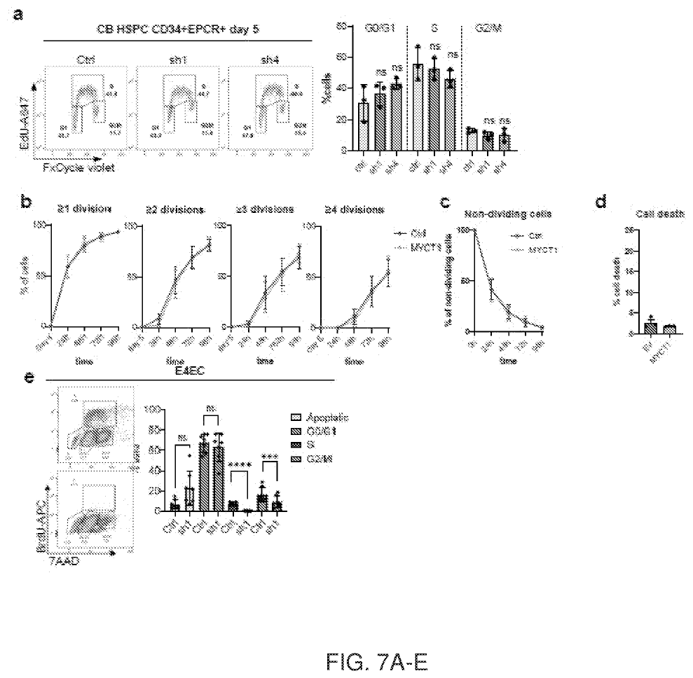

Since MYCT1 KD disrupted the expression of cell division and HSC proliferation genes and prevented HSPC expansion and engraftment, the inventors investigated if MYCT1 KD HSPCs show defective proliferation. CFSE proliferation experiments using sorted HSPC and daily monitoring of single cell divisions revealed slower proliferation kinetics an increased proportion of non-dividing cells, and a slight increase in cell death after MYCT1 KD, without significant effects on cell cycle distribution at day 5, while MYCT1 OE HSPC proliferated normally (FIGS. 5H-5K, FIGS. 7B-7D). MYCT1 KD in endothelial cells, which also impairs their expansion in culture, disrupted cell cycle progression (FIG. 7E).

### MYCT1 is an Endosomal Protein

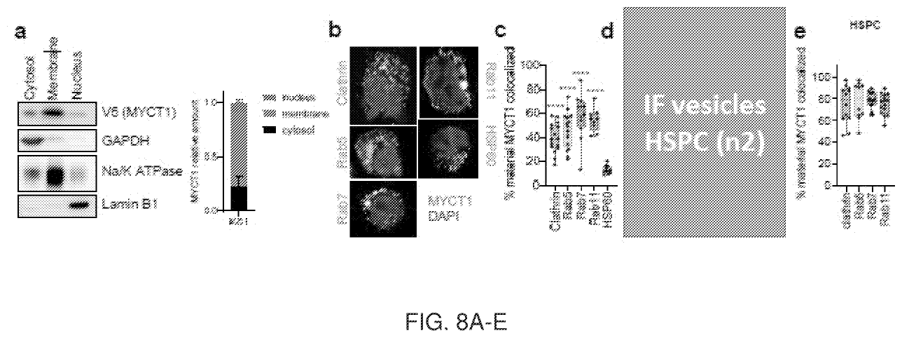

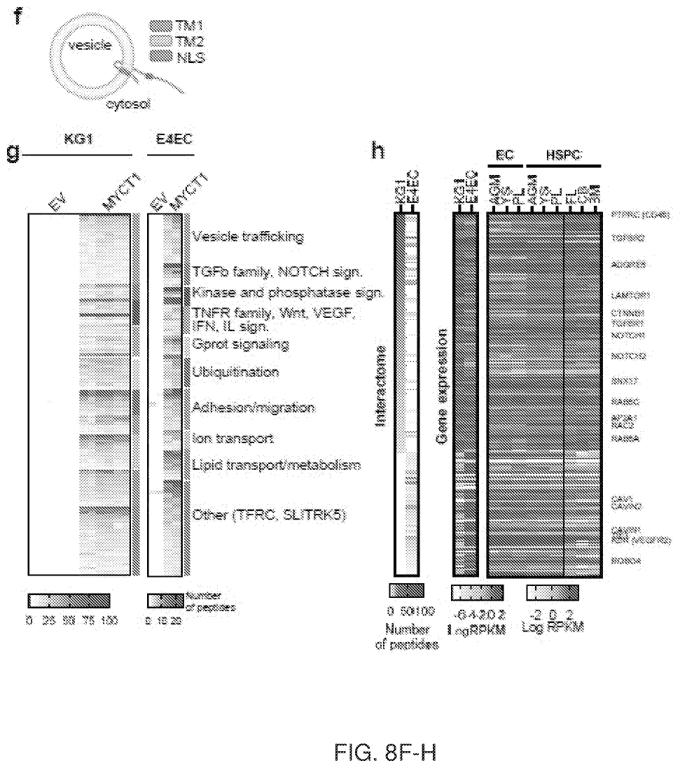

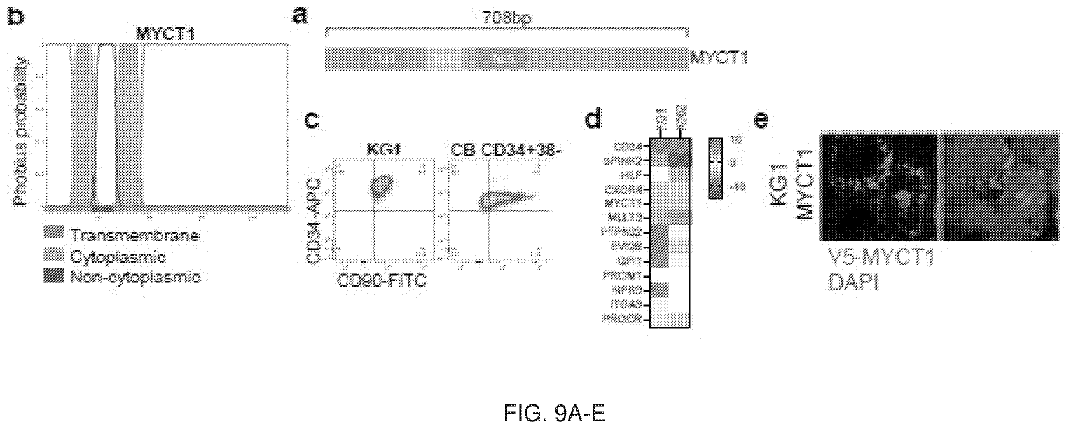

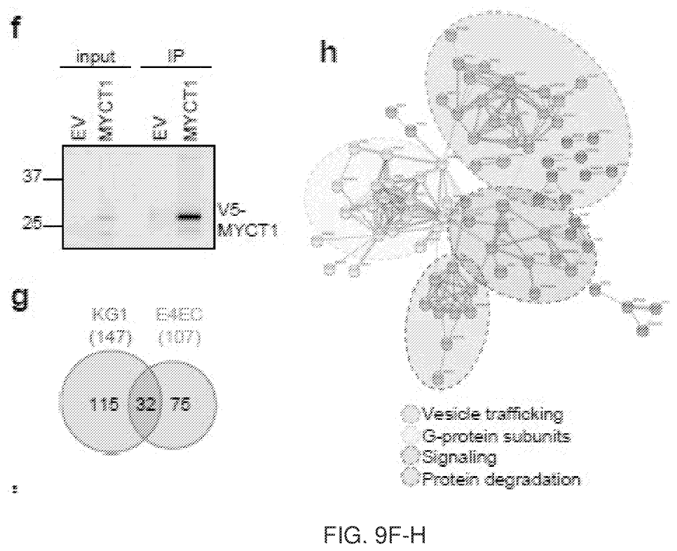

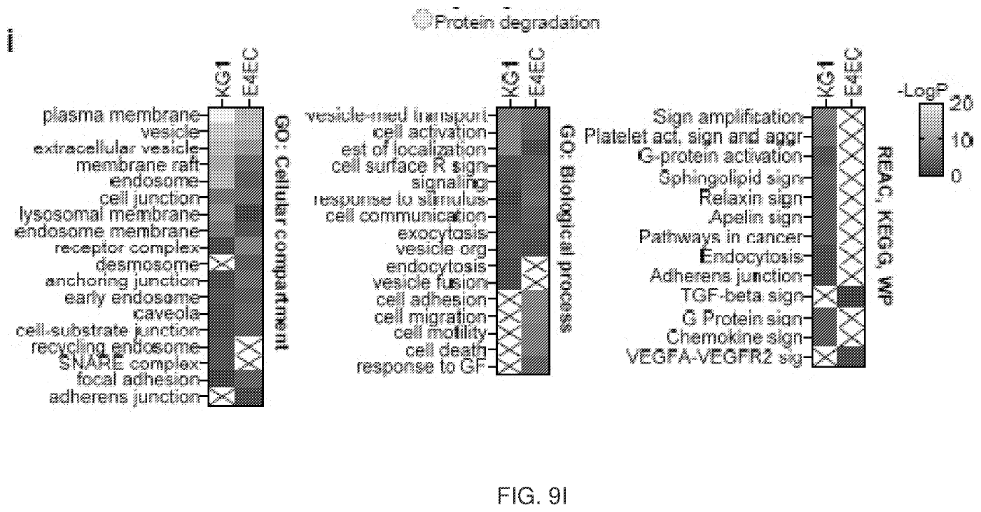

MYCT1 has been described both as a nuclear transcription factor and as a membrane protein, but its structure and molecular function have not been defined in hematopoietic cells. MYCT1 has two transmembrane domains and a putative nuclear localization signal, and topology prediction predicted the N- and C-terminal ends to be cytoplasmic, with a short non-cytoplasmatic region between the two TM domains (FIGS. 9A and 9B). The inventors characterized the localization of V5-tagged MYCT1 in KG1 cells, which show HSC-like surface phenotype (CD34+CD90+) and gene expression (FIGS. 9C and 9D), and found that MYCT1 is enriched in the membrane fraction and localizes in vesicle-like structures in the cytoplasm. Co-staining with endosomal markers in KG1 cells and human CB HSPCs revealed MYCT1 co-localization with clathrin (structurally responsible for the formation of coated vesicles in the initial steps of endocytosis) and the endosomal proteins RAB5 (early endosomes), RAB7 (late endosomes), and RAB11 (late endosomes). No colocalization was found with HSP60, a mitochondrial marker used as a negative control (FIGS. 8A-8F, FIG. 9E).

### MYCT1 Interacts With Vesicle Trafficking and Signaling Components

To elucidate the molecular function of MYCT1 the inventors performed immunoprecipitation coupled with high sensitivity mass spectrometry (IP-MS) of V5-tagged MYCT1 in KG1 hematopoietic cells and E4ECs (FIG. 9F). The inventors found that MYCT1 interacts with numerous vesicle trafficking components (e.g. Rab5, AP2A1) and signaling receptors (e.g. TGFBR1-2, NOTCH1-2, CTNNB1), as well as G-protein subunits, protein degradation machinery, adhesion and migration proteins (e.g. RAC2), lipid and ion transporters (FIG. 8G). STRING analysis showed that MYCT1 interactors are part of shared functional and/or physical networks (FIG. 9H), and the MYCT1 interacting proteins identified in KG1 cells and E4ECs belong to same protein families, although only around 20% of the interactors were detected in both cell types (FIG. 8G and FIG. 9G). GO term analysis of MYCT1 interactors revealed a significant enrichment for plasma membrane and endosomal proteins, and for proteins involved in vesicle-mediated transport and cell surface receptor signaling. Pathway analysis showed a significant association with signal amplification, pathways in cancer, endocytosis and chemokine signaling pathways in KG1 cells, and TGF-β and VEGFR2 signaling in E4EC (FIG. 9H). To evaluate if the MYCT1 interactors identified in the cell lines are relevant to human HSPC biology, the inventors analysed the gene expression of the MYCT1 interactors in KG1 and E4EC cell lines, as well as in endothelial cells and HSPCs from 5 week human AGM, yolk sack (YS), and placenta (PL), and HSPCs from 2^{nd} trimester FL, neonatal CB and adult bone marrow (BM). The interactome genes from KG1 cells were expressed not only in KG1 cells, but also in FL, CB and BM HSPCs, while the MYCT1 interactors specifically detected in E4EC were mostly absent in KG1 cells, but expressed in E4EC and the ECs from 5 week AGM, YS and PL. HSPCs from the 1st trimester tissues, which are less mature. These tissues expressed many of the EC-specific interactors and showed an expression pattern between the KG1 and mature HSPCs, and E4EC and embryonic ECs (FIG. 8H). These data show that MYCT1 has a function in endosomes and interacts with the vesicle trafficking network and signaling components relevant for different stages of HSC and EC biology, and confirm KG1 and E4EC as suitable models for studying MYCT1 function in human HSPCs throughout development.

### MYCT1 Controls Endocytosis

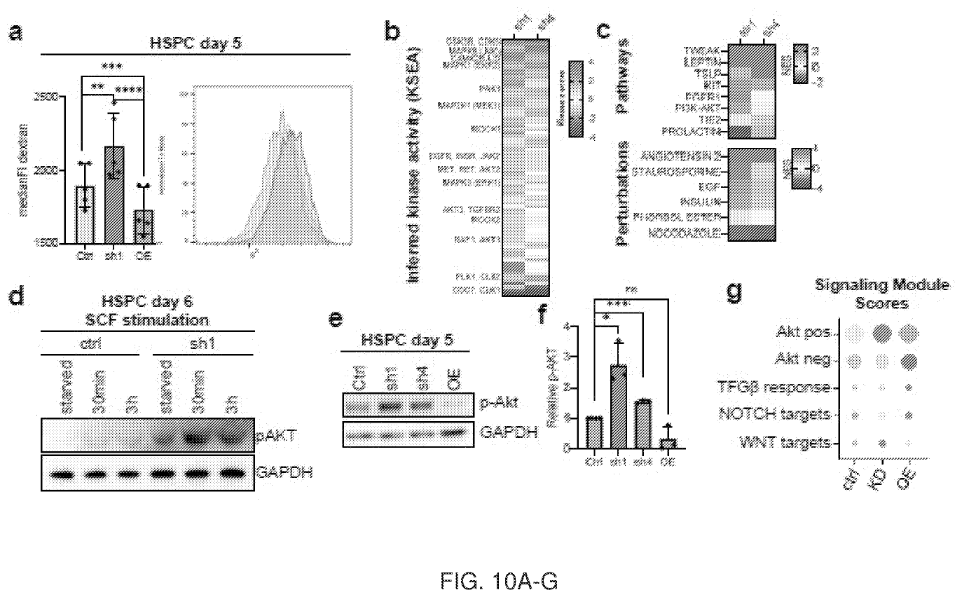

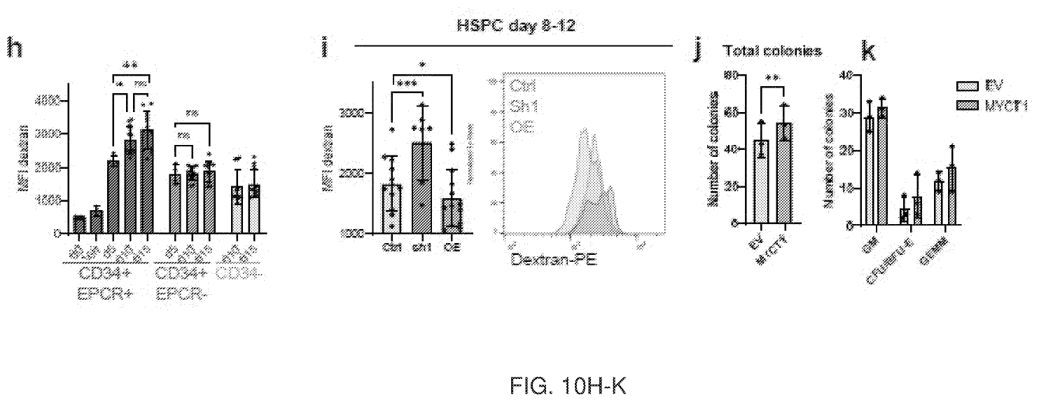

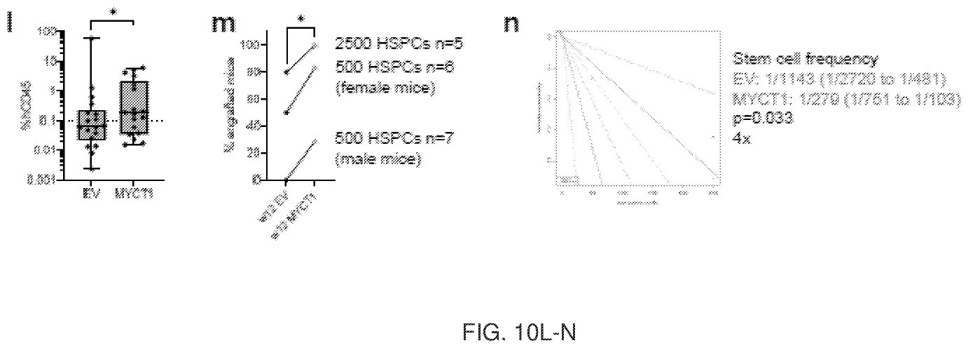

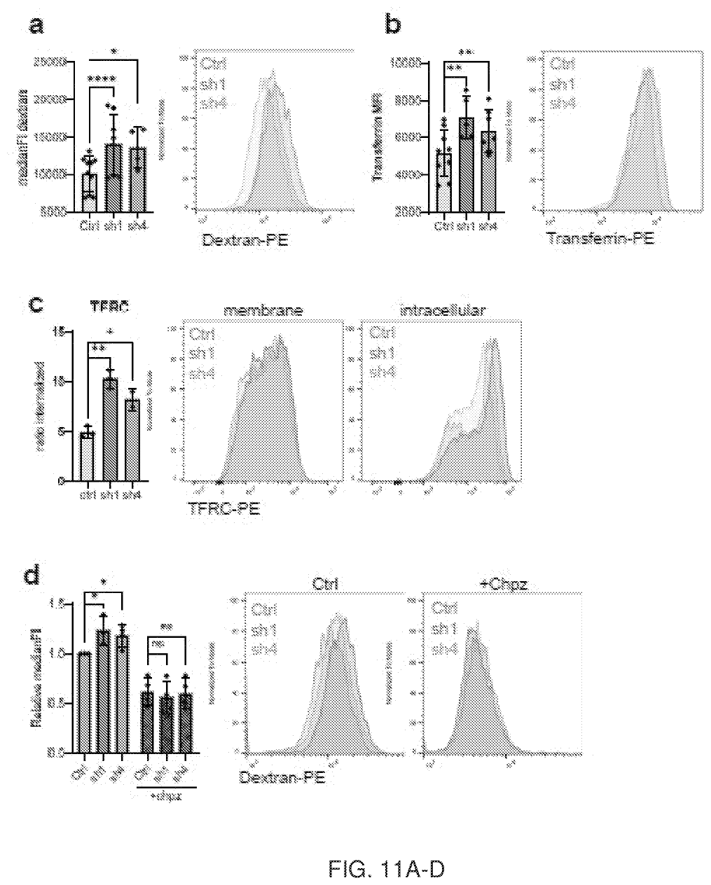

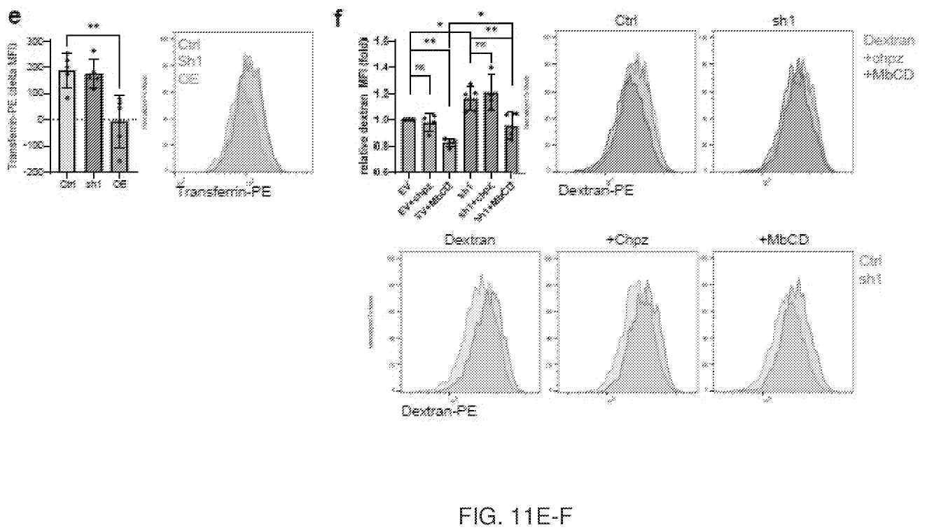

Since the inventors observed that MYCT1 localizes in endosomes and interacts with endosomal and vesicle trafficking proteins, the inventors asked if MYCT1 has a function in the regulation of endocytosis. Quantification of internalization of fluorescent dextran in E4ECs showed an increase in endocytosis after MYCT1 KD. MYCT1 KD also increased the internalization of fluorescently-labelled transferrin, and the transferrin receptor (TFRC), which are known to be internalized through the clathrin-mediated endocytosis (CME) pathway, the major route of internalization. Pre-treatment of E4EC with chlorpromazine, an inhibitor of CME, dampened the increased endocytosis caused by MYCT1 loss, confirming that MYCT1 regulates CME in endothelial cells (FIGS. 11A-11D). Similarly, MYCT1 KD in human CB HSPCs also led to increased internalization of dextran, while MYCT1 OE decreased it (FIG. 10A). MYCT1 OE in CB HSPCs also dampened transferrin internalization, suggesting an effect on CME. However, MYCT1 KD had no effect on transferrin internalization, and pre-treatment with chlorpromazine, nor methyl-beta-cyclodextrin (MbCD), a cholesterol-depleting drug used to inhibit caveolin-mediated endocytosis, were not sufficient to restore hyperactive endocytosis due to MYCT1 loss (FIGS. 11E and 11F). This could suggest that hyperactive internalization induced by MYCT1 KD is not reversible or can take place through chlorpromazine-resistant mechanisms in HSPCs.

### MYCT1 Loss Causes Hypersensitivity to Microenvironmental Signals

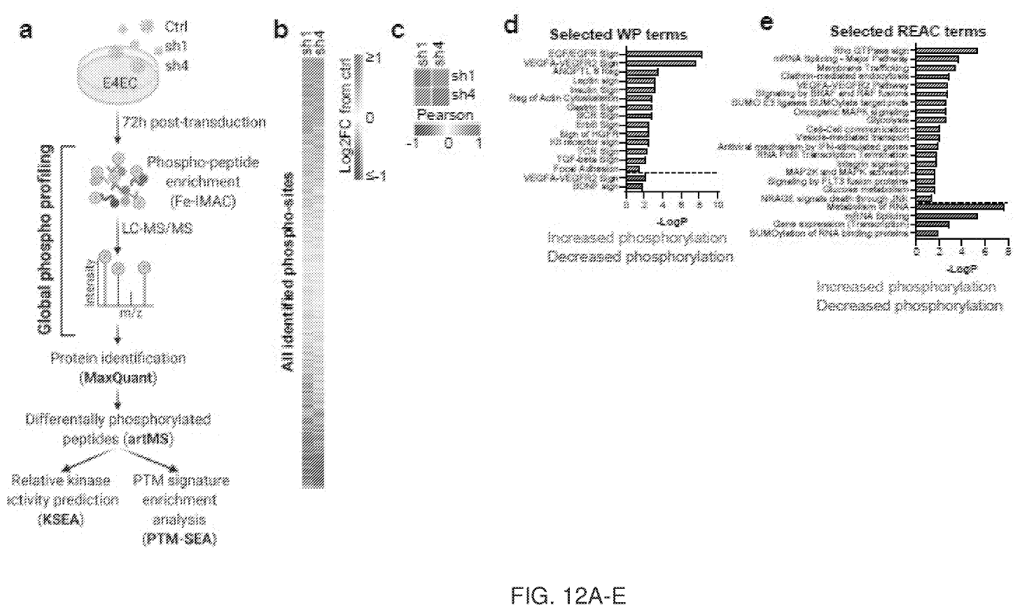

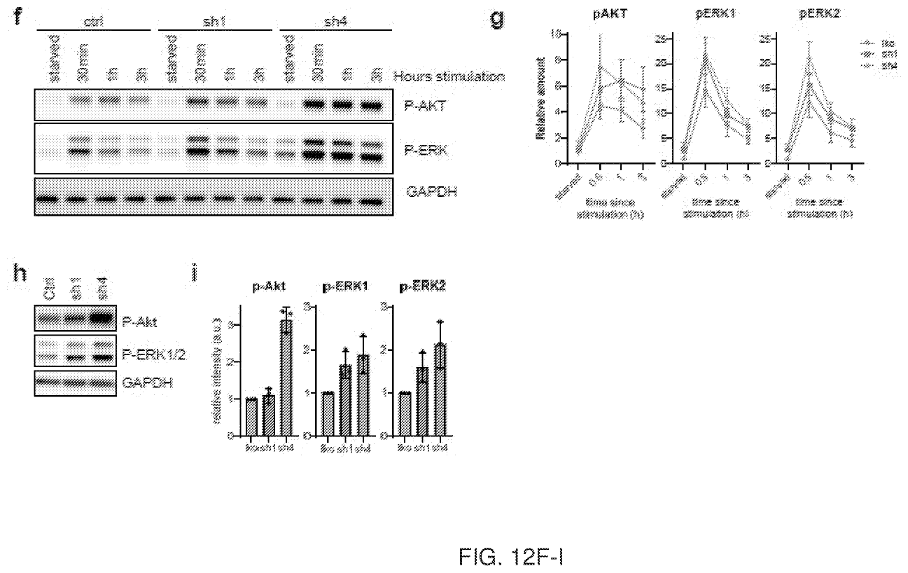

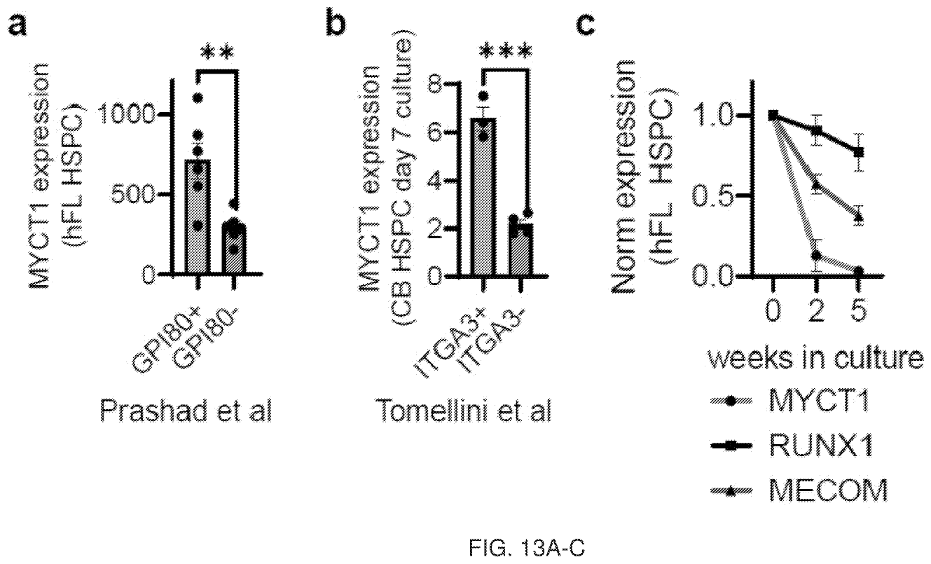

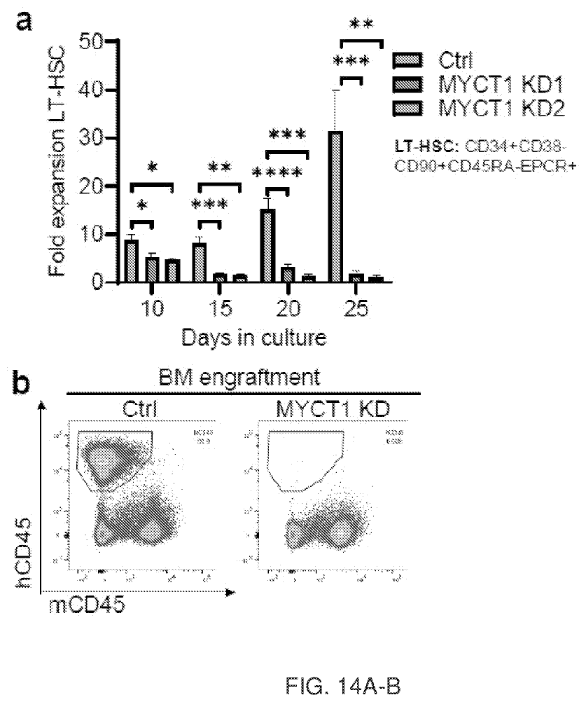

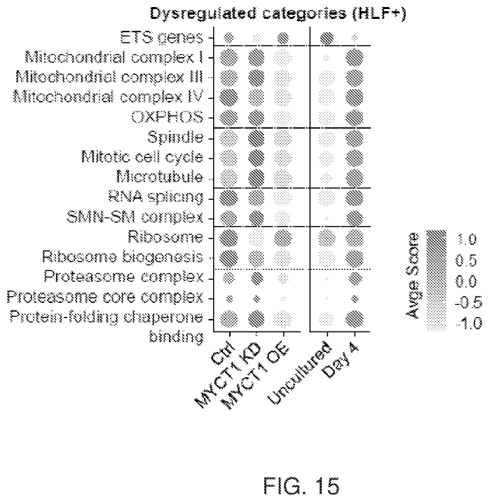

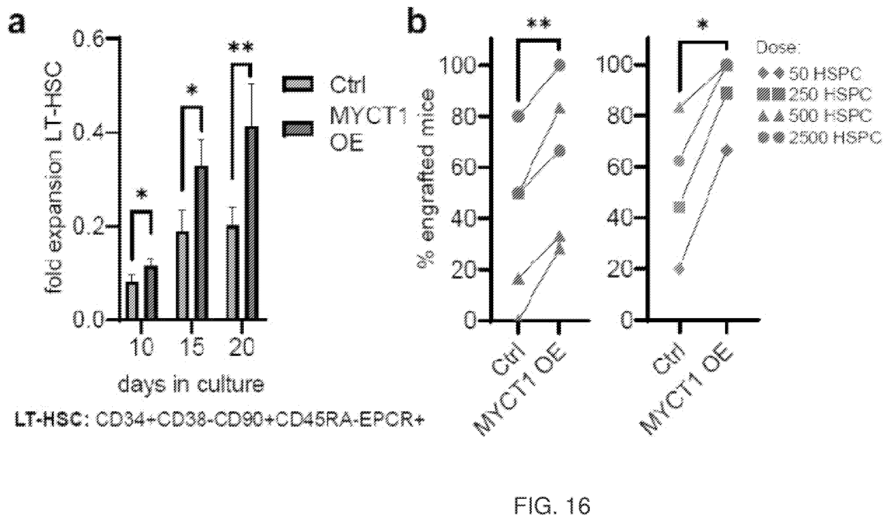

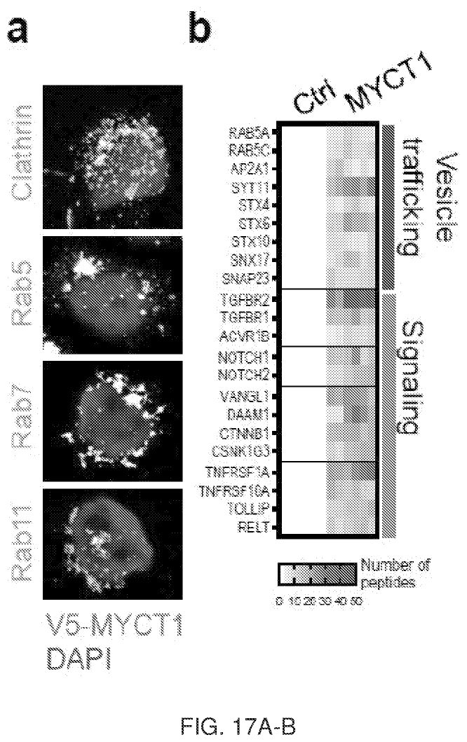

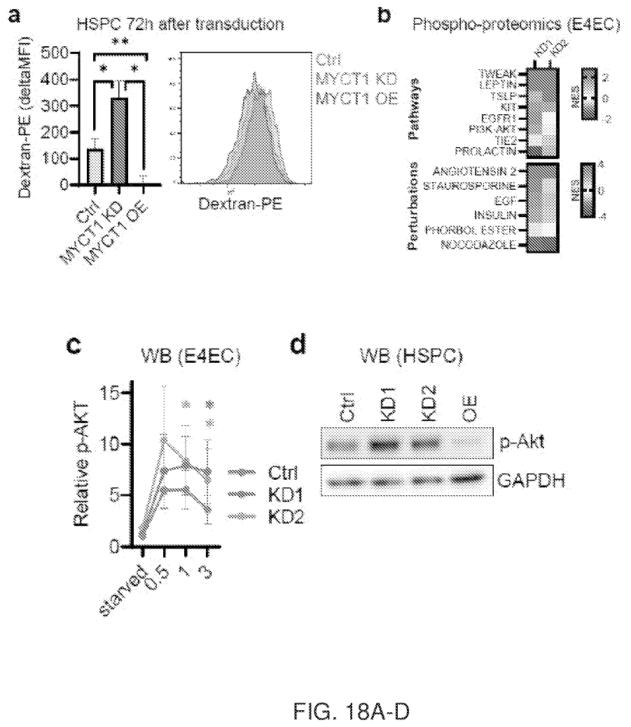

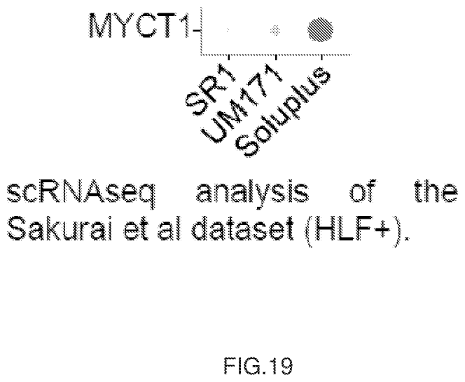

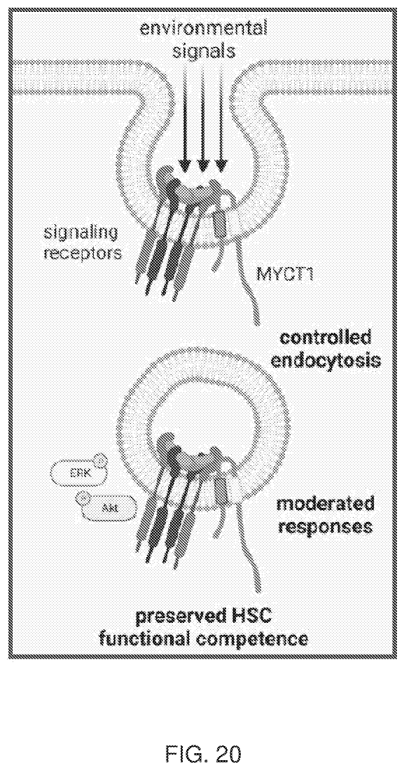

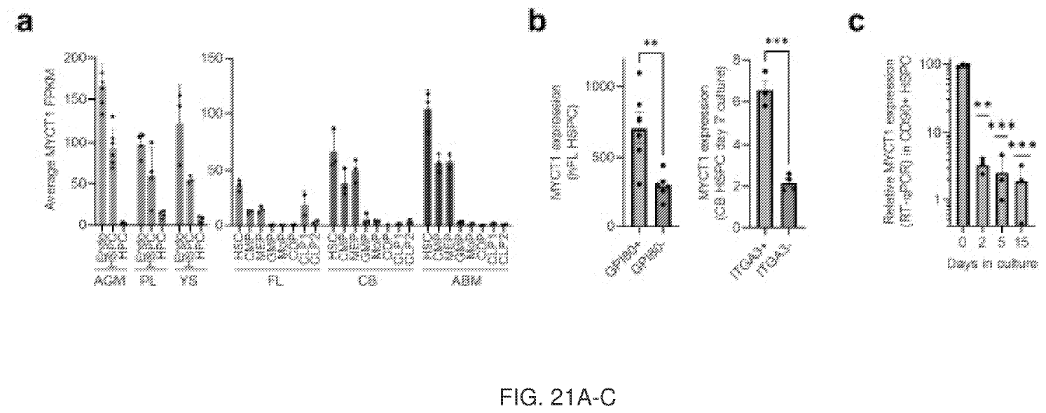

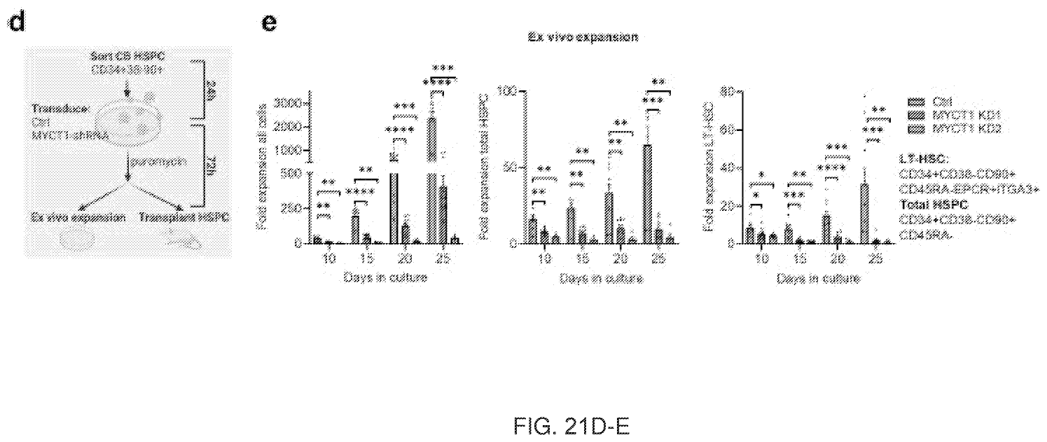

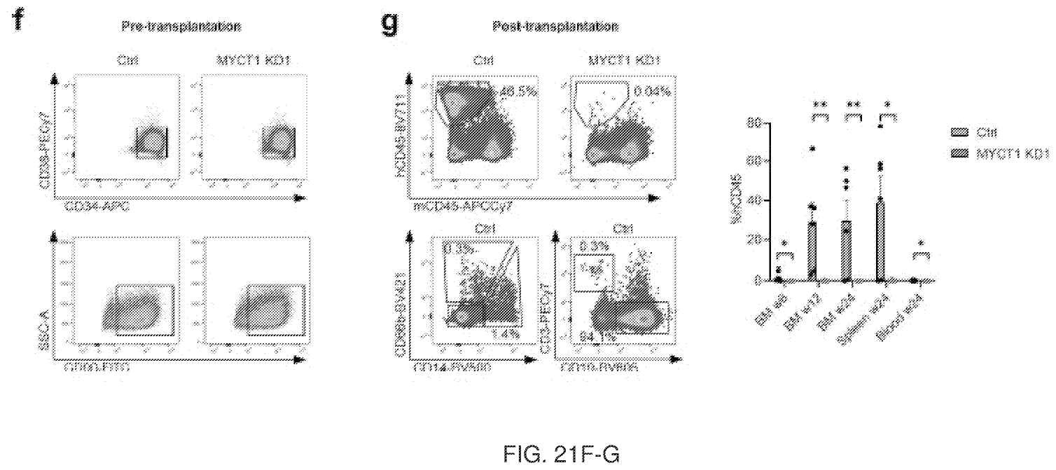

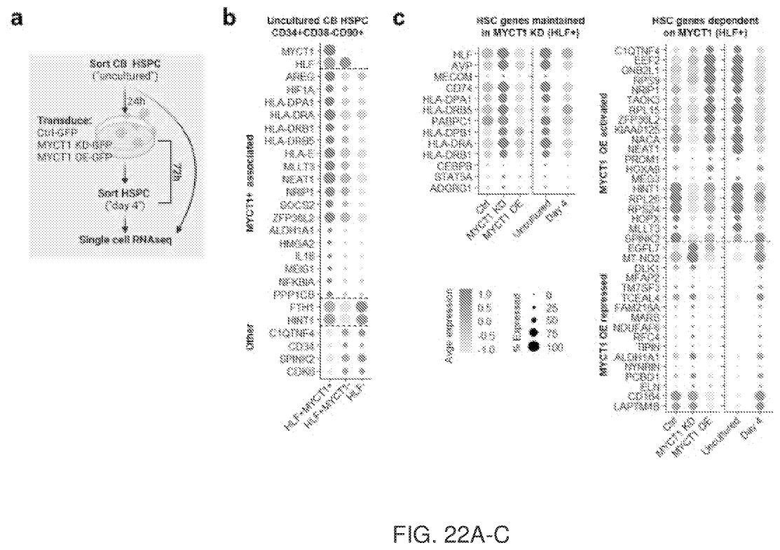

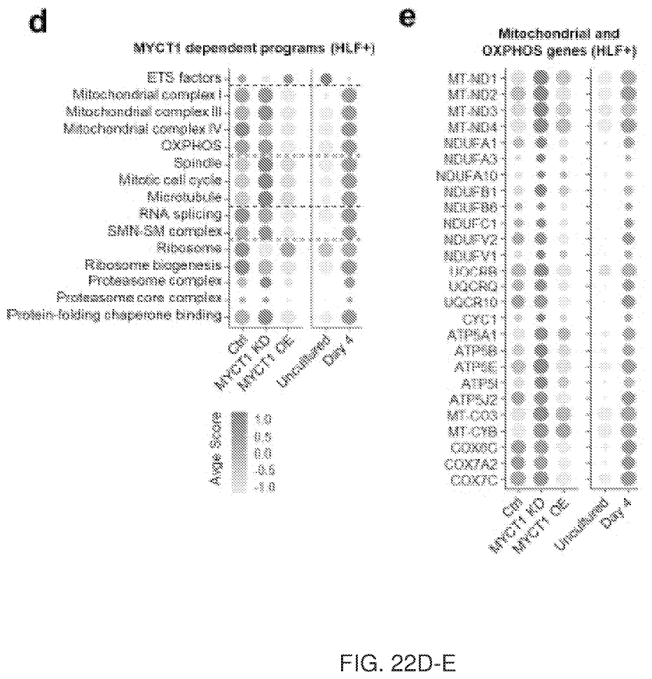

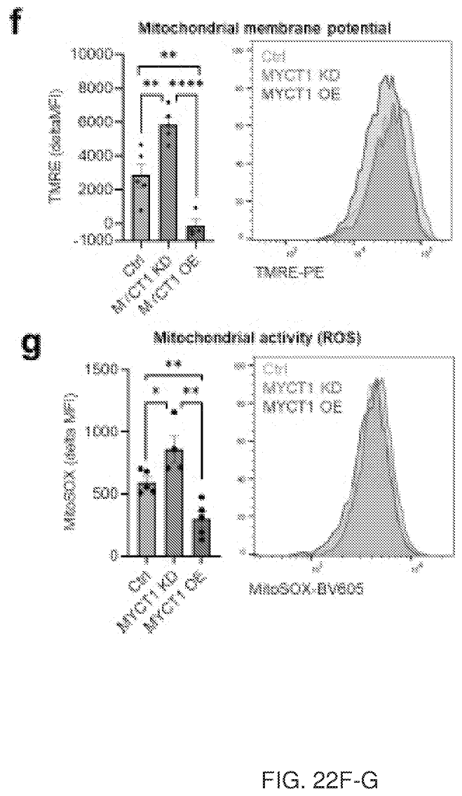

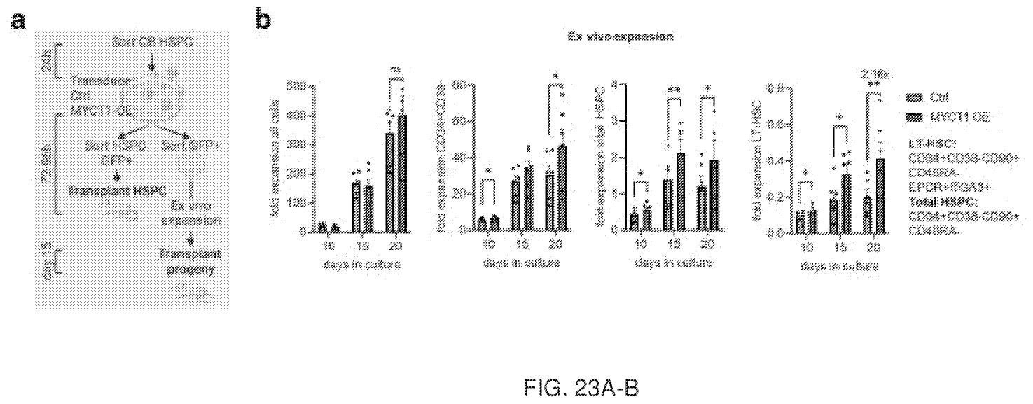

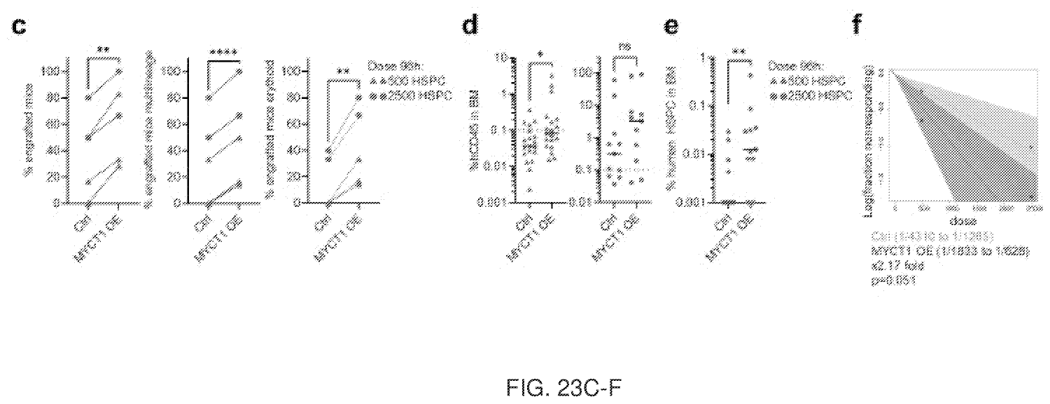

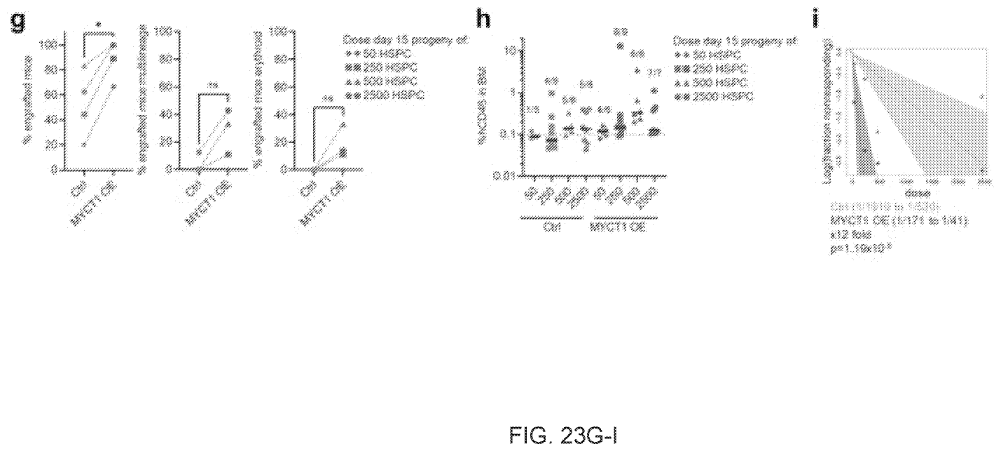

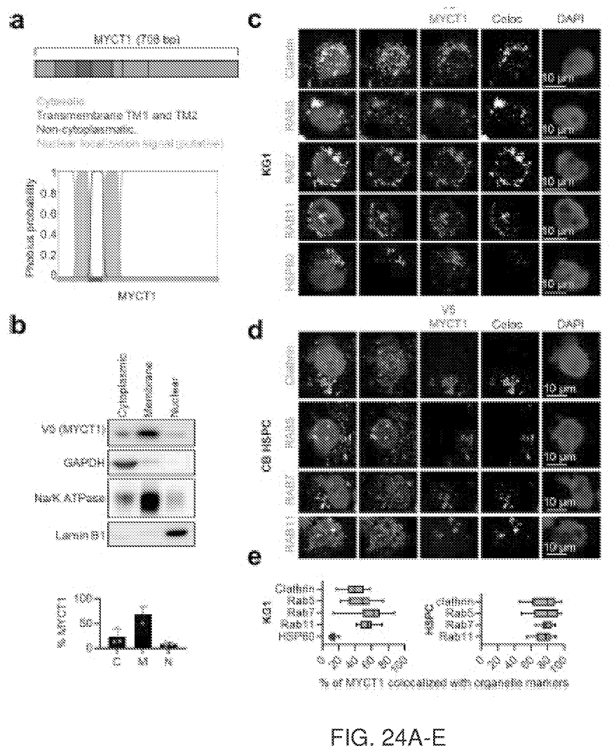

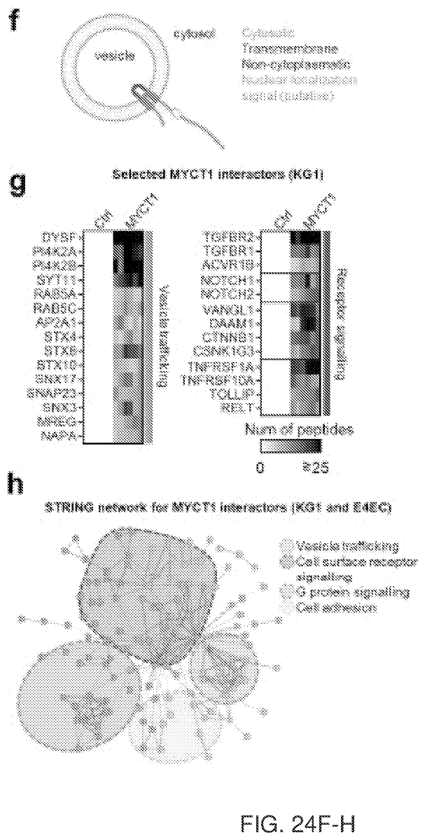

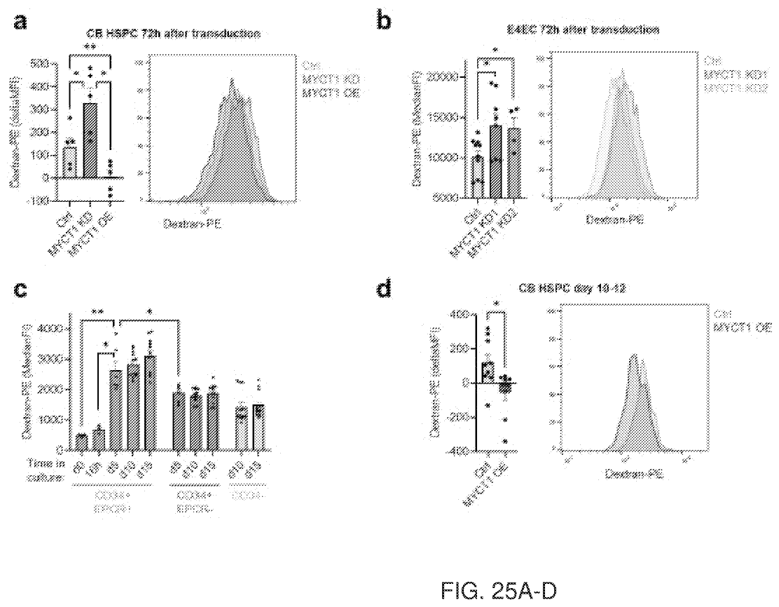

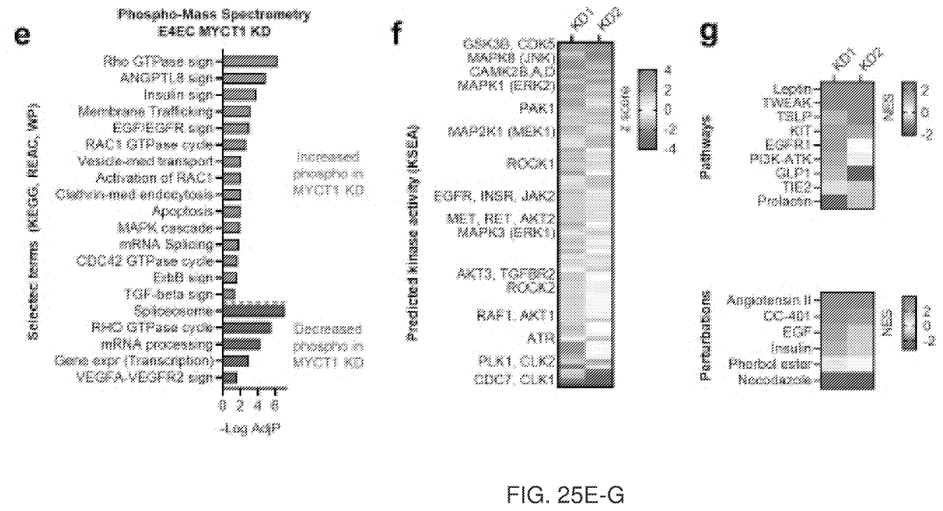

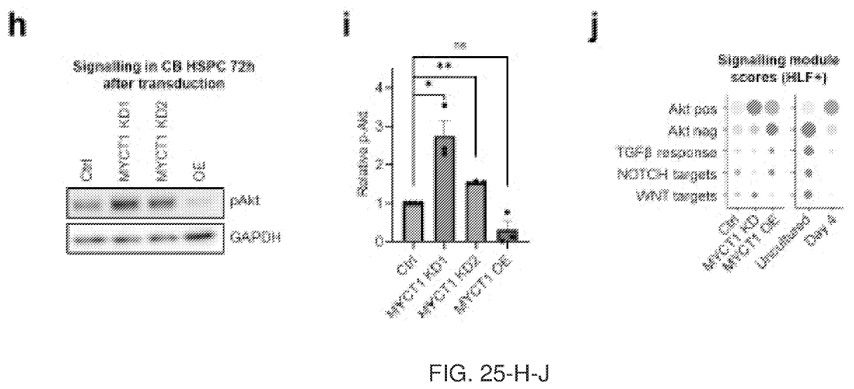

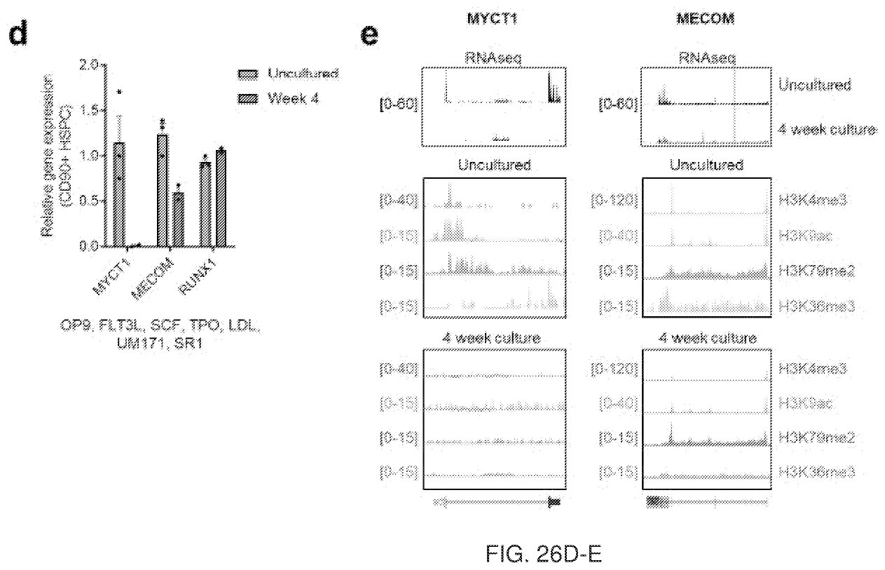

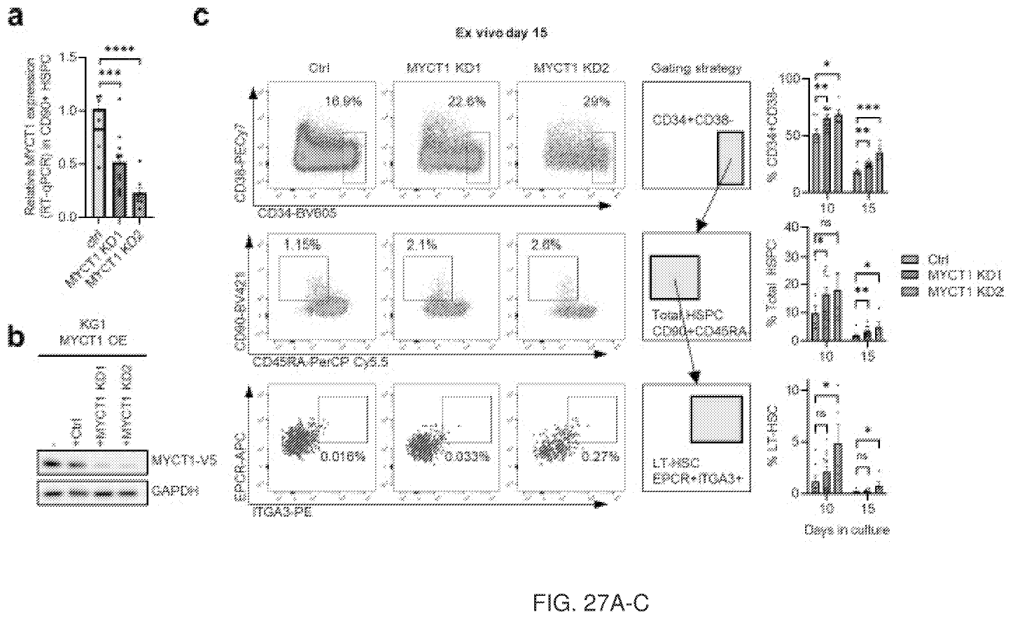

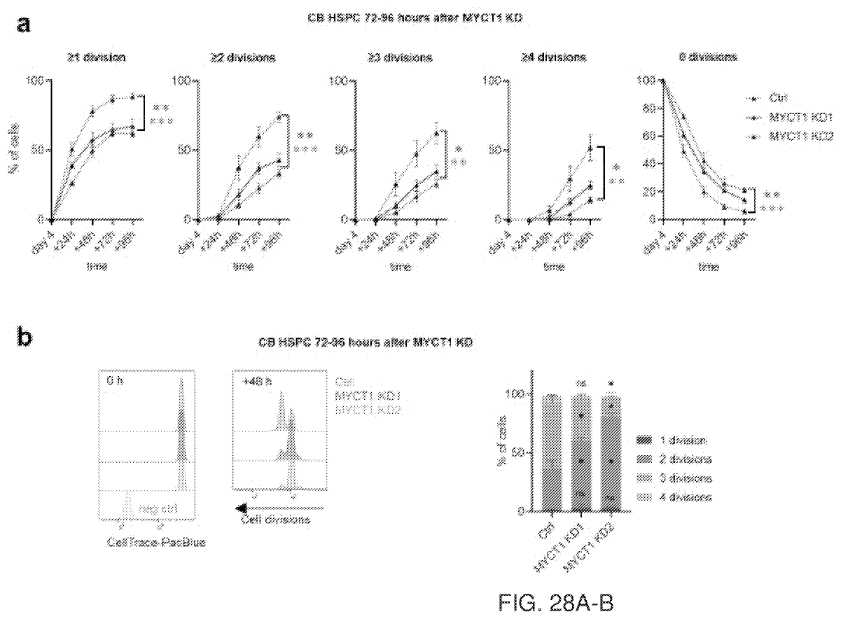

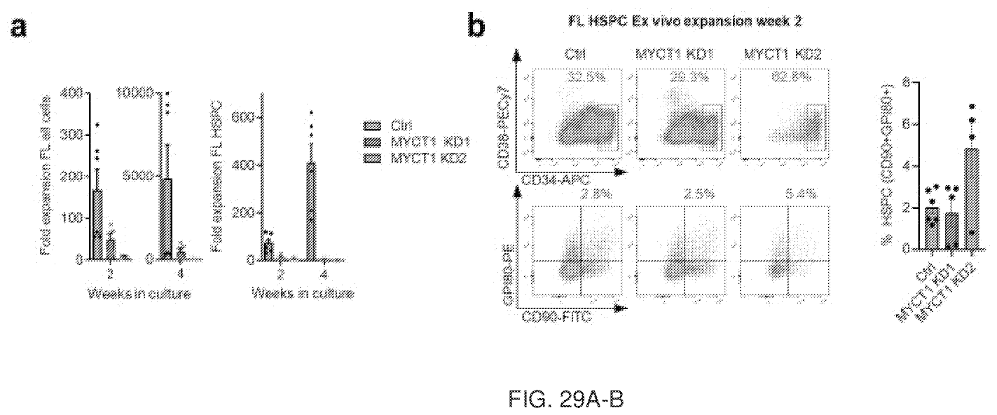

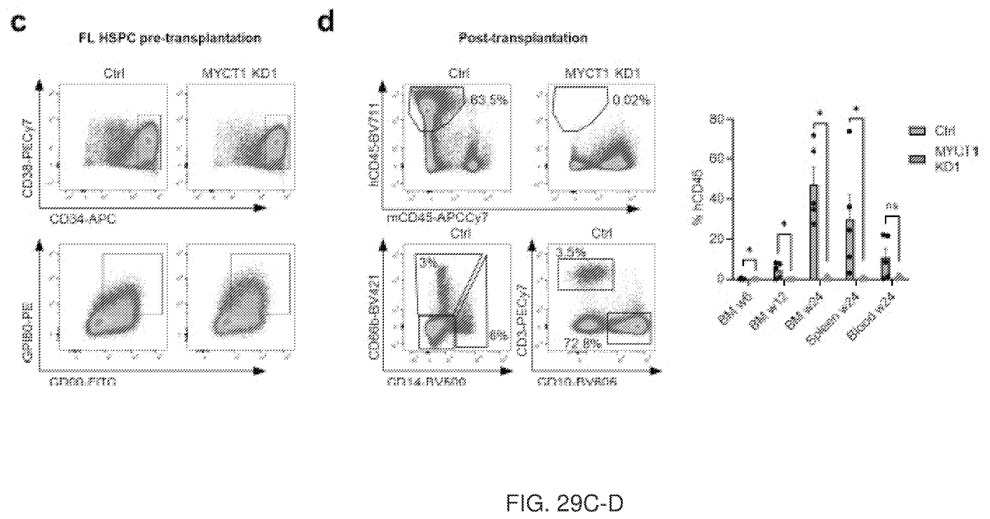

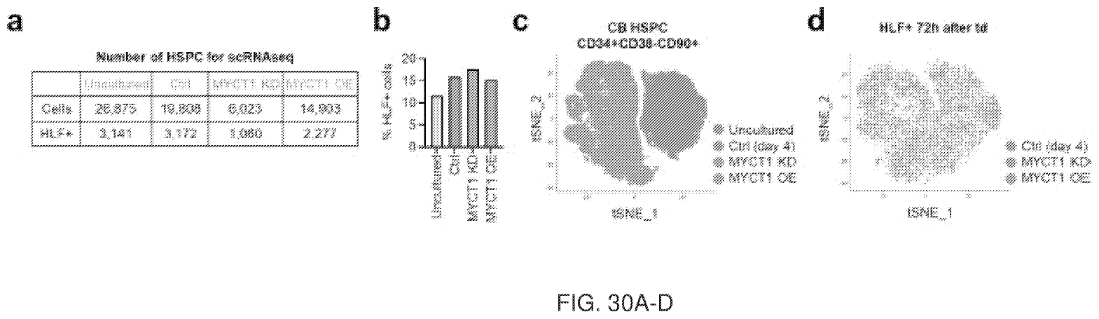

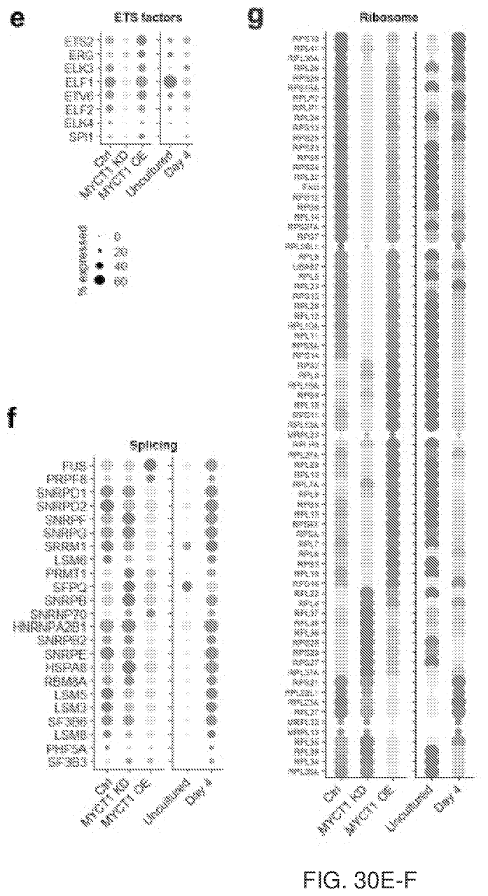

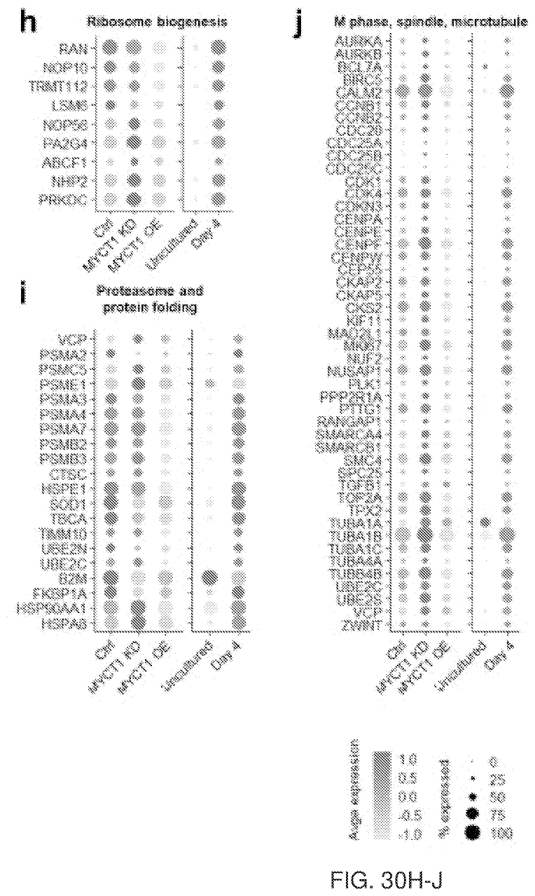

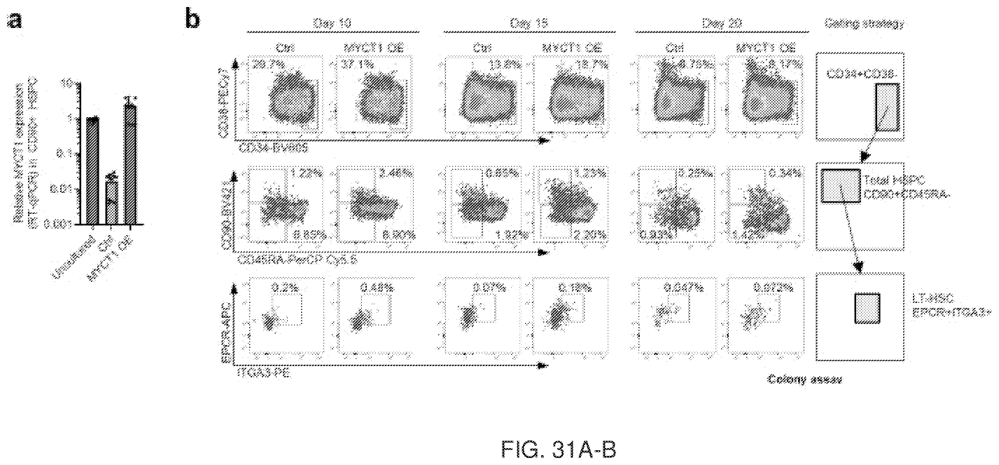

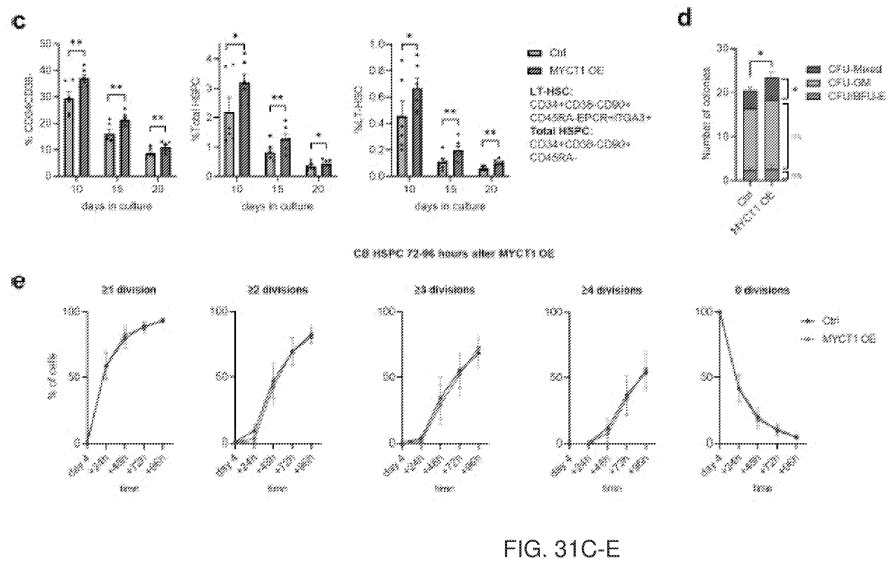

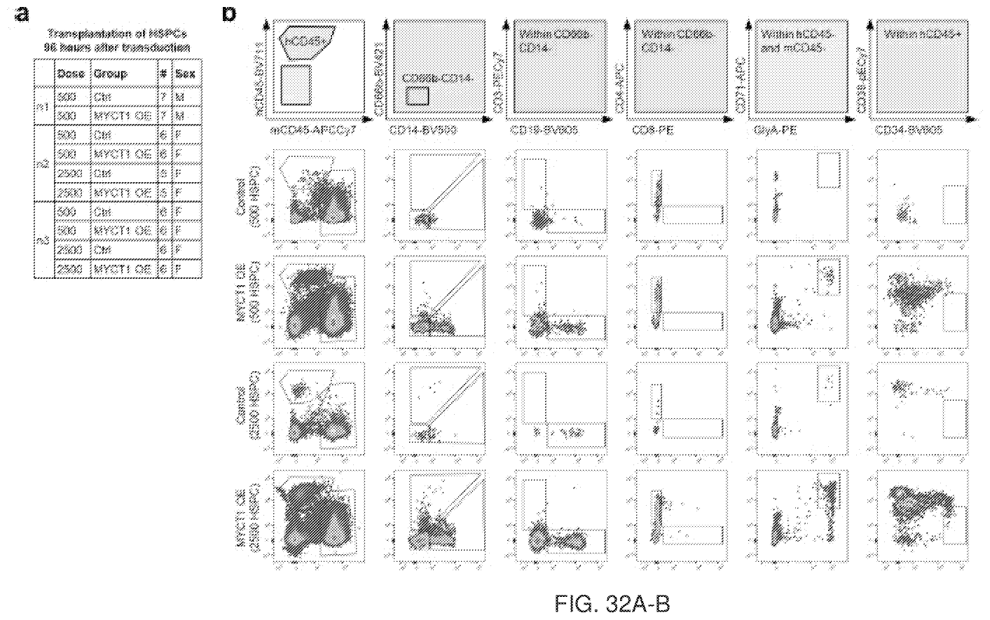

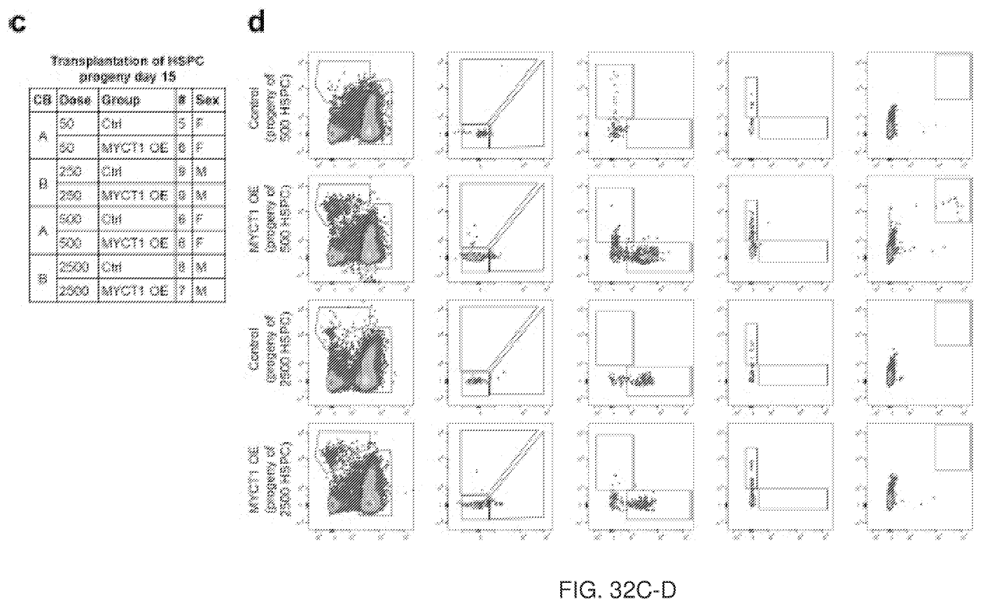

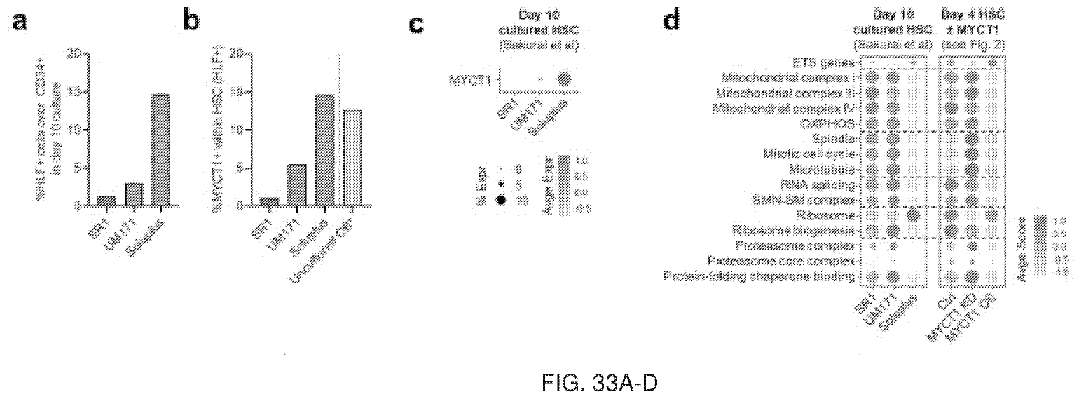

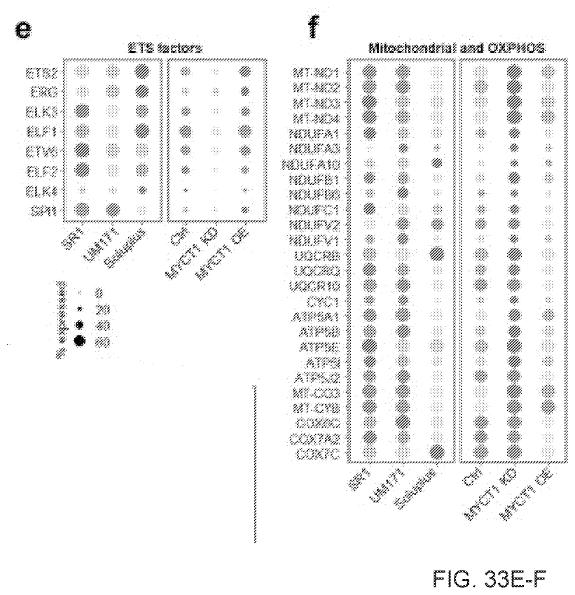

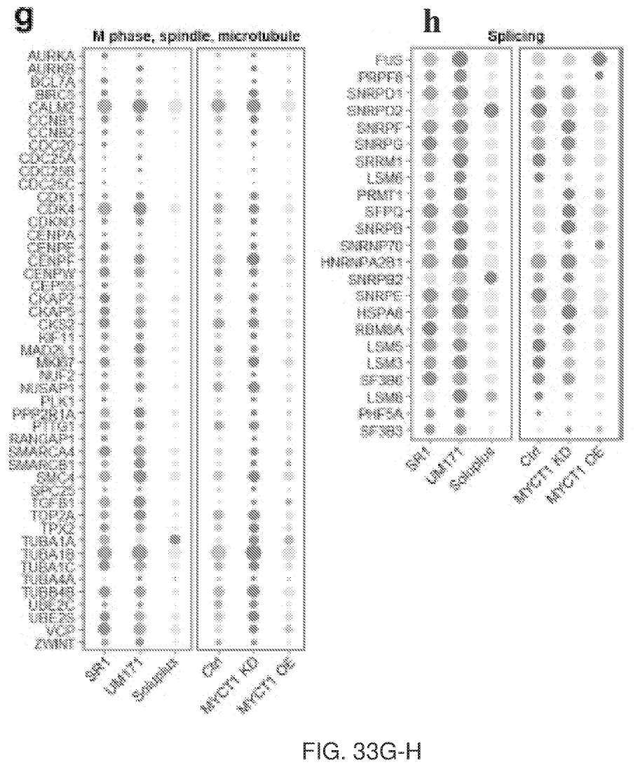

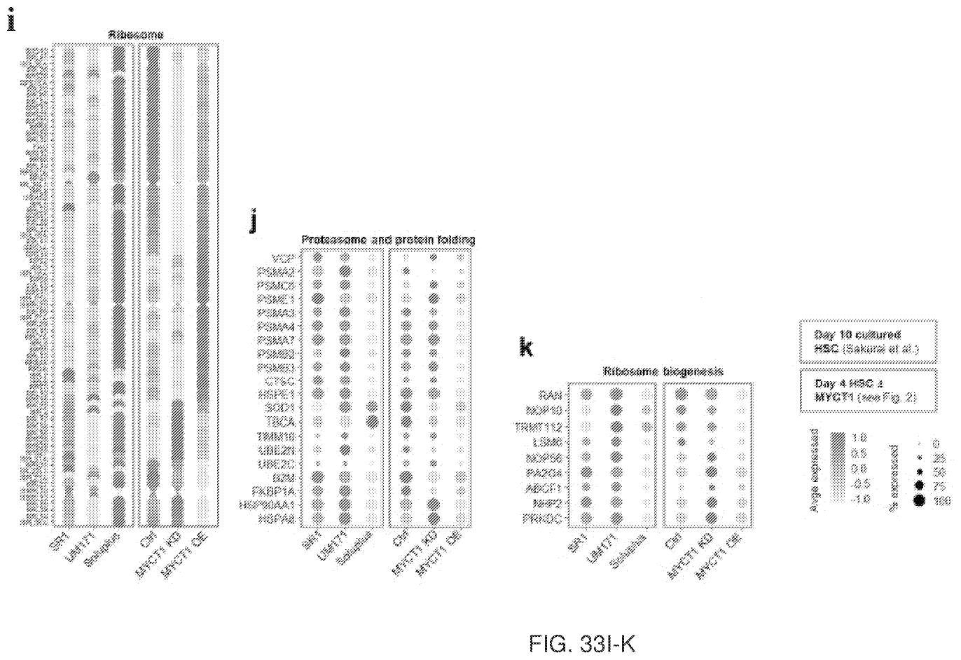

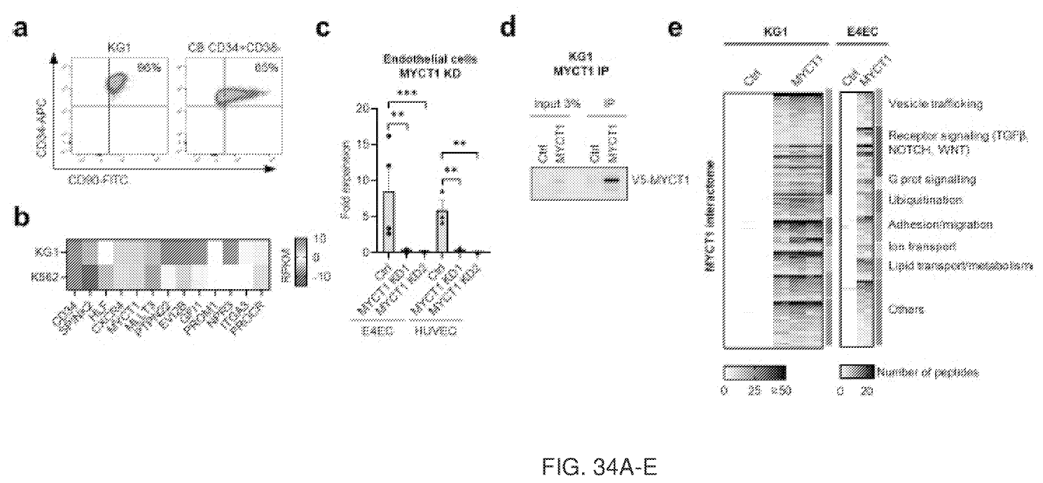

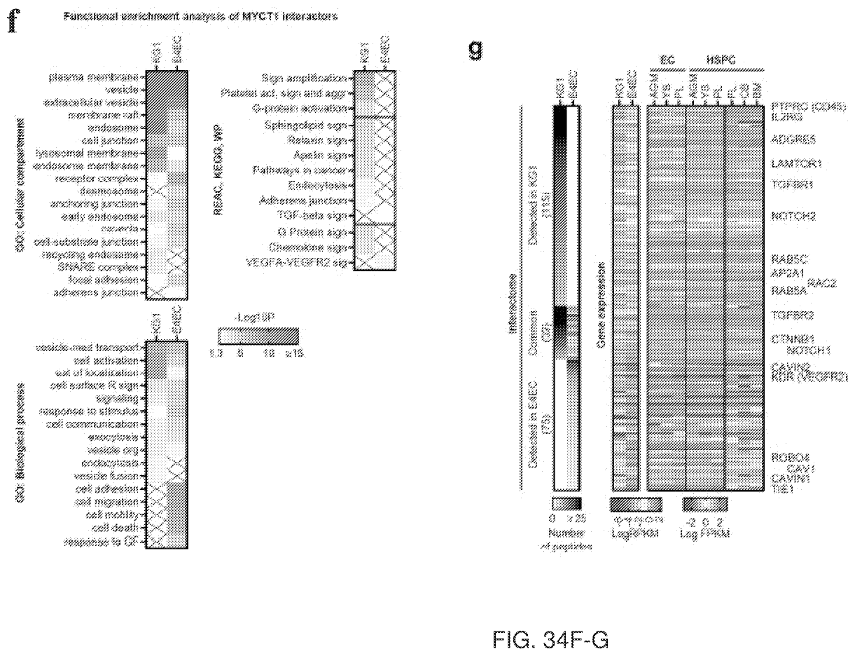

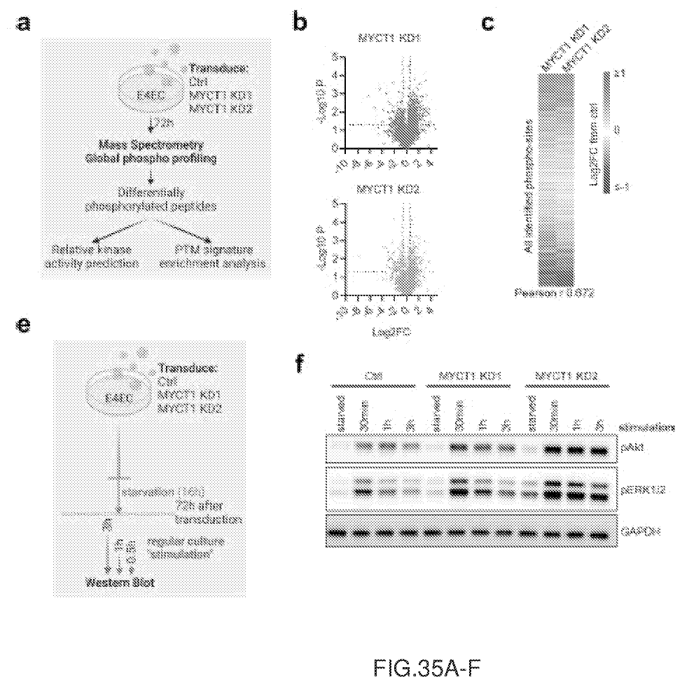

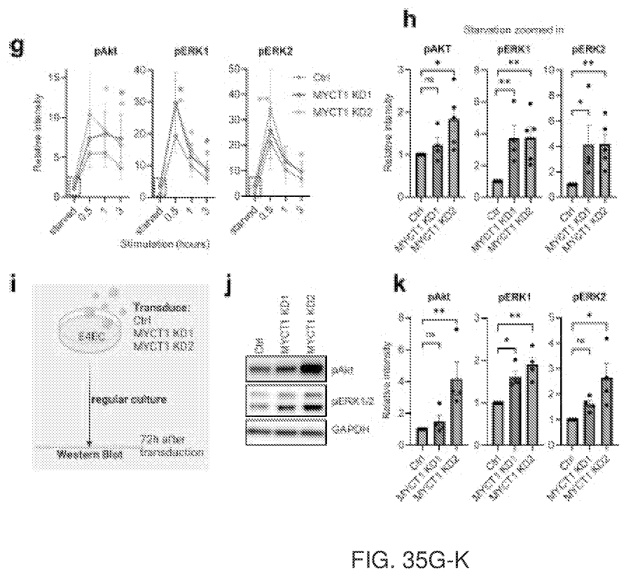

Endocytosis is a critical regulatory step in extracellular signaling that can determine the length, strength, and quality of cell signaling from numerous receptors and must be tightly controlled to maintain adequate responses to external signals. To determine if MYCT1 loss leads to a disruption of signaling pathways, the inventors performed mass spectrometry-based global phospho-proteomic profiling in control and MYCT1 KD E4ECs, combined with analysis of differentially phosphorylated peptides, relative kinase activity prediction (red), and signature enrichment analysis (FIG. 12A). MYCT1 KD with 2 different shRNAs led to global changes in the phospho-proteome, with increased phosphorylation of proteins involved in signaling from EGFR, VEGFR2, Insulin, or Rho-GTPases, as well as proteins involved in membrane trafficking, CME, or glycolysis among others (FIGS. 12B-12E).

Because these analyses are performed in a gene-centric manner and do not consider the specific modified sites, the inventors next used the site-specific information to predict relative kinase activity (KSEA). This revealed a hyperactivation of multiple kinases involved in signal transduction (e.g. MAPKs such as JNK, MEK1 and ERK1/2, EGFR, INSR), endocytosis (CDK5, PAK1), and cell cycle arrest (CHECK1, CDK5) in response to MYCT1 loss. On the other hand, kinases involved in cell cycle progression, gene splicing regulation, and DNA damage control (e.g. CDC7, PLK1, CLK1/2, ATR) had decreased predicted activity (FIG. 10B). Phosphosite-centric signature enrichment analysis also revealed increased activation of pro-angiogenic signaling pathways (TWEAK, Leptin and TLSP (thymic stromal lymphopoietin)), PI3K-AKT signaling, and inhibition of TIE2 and prolactin signaling, which are essential for endothelial cell survival, proliferation, adhesion and migration. Interestingly, MYCT1 KD E4ECs showed increased signatures consistent with responses to EGF and insulin treatment, although both control and KD cells are cultured in equal concentrations of EGF and IGF1 (insulin growth factor 1) (FIG. 10C). This data show that MYCT1 loss leads to global hyperactivation of signaling pathways, and suggest an increased sensitivity to the extracellular signals present in the culture media. Indeed, time course western blot experiments after overnight starvation show slightly increased AKT and ERK phosphorylation under starved conditions, and increased and prolonged AKT and ERK phosphorylation in response to stimulation with complete media, which leads to a basal hyperactivation of those pathways when the cells are cultured normally with complete media (FIGS. 12F-12I).

Similarly, MYCT1 KD human CB HSPCs show increased AKT phosphorylation after overnight starvation and in response to SCF stimulation, and a basal hyperactivation when the cells are cultured in normal conditions (SCF, FLT3, TPO) (FIGS. 10D-10F). Furthermore, module score analysis of scRNAseq data reflects the hyperactivation of AKT signaling in MYCT1 KD HSC, and suggests that responses to TGFβ and NOTCH are increased while the response to WNT is decreased, with MYCT1 OE having the opposite effect (FIG. 10G).

### Endocytosis Increases in Culture and can be Restored by MYCT1 OE to Improve HSC Stemness

Since the inventors found that MYCT1 becomes downregulated in cultured HSPCs and is required to moderate endocytosis, the inventors asked if endocytosis increases during HSPC culture and if it contributes to the HSC “culture defect”. Indeed, dextran internalization assays showed that cultured HSPCs (CD34+EPCR+), but not the differentiated progenitors (CD34+EPCR− and CD34− cells), gradually increase endocytosis over time (FIG. 10H). Strikingly, MYCT1 OE was able to dampen endocytosis in HSPCs also after prolonged time in culture (FIG. 10I). Moreover, restoring MYCT1 expression in cultured HSPCs via lentiviral OE improved the expression scores for HSC hallmarks and restored transcriptomic programs disrupted in culture and further disrupted by MYCT1 KD (FIGS. 5A-K, FIGS. 6A-6D). Therefore, the inventors investigated if MYCT1 OE was able to improve the function of cultured human HSPCs by assessing colony formation and HSC engraftment ability. Cultured MYCT1 OE HSPCs sorted (CD34+CD38−CD90+GFP+) after 5 days in culture gave rise to more colonies, and specifically increased the number of mixed (granulocyte, erythroid, monocyte and macrophage, GEMM) and erythroid colonies (FIGS. 10J and 10K). Finally, transplantation of equal number of sorted HSPCs after 5 days in culture with control or MYCT1 OE showed increased levels of human engraftment and increased number of engrafted mice after MYCT1 OE, resulting in a 4-fold increase in the calculated stem cell frequency (1/1143 for control and 1/279 for MYCT1) (FIGS. 10L-10N). These data indicate that restoring MYCT1 expression in cultured human CB HSPCs ameliorates the hyperactivation of endocytosis and signaling pathways, and can improve HSC function.

### Example 2—MYCT1 Moderates Environmental Sensing Via Endocytosis to Preserve Human Hsc Self-Renewal

Ex vivo expansion of functional human hematopoietic stem cells (HSC) would greatly improve the access to HSC transplantation therapies for blood disorders. However, the programs governing human HSC self-renewal and engraftment ability are poorly understood, and cannot be recapitulated in culture. The inventors discovered a novel HSC regulatory gene, MYCT1 (MYC target 1), that is selectively expressed in endothelial cells (EC) and undifferentiated human HSPCs but becomes drastically downregulated during HSC ex vivo culture. Knockdown (KD) experiments revealed that MYCT1 is critical for human HSC expansion and engraftment. Single cell RNAseq in MYCT KD and overexpression HSC cultures indicated that MYCT1 governs transcriptional signatures associated with HSC identity, as well as biological processes essential for HSC stemness, such as tightly controlled mitochondrial activity or proteostasis. Whereas the loss of MYCT1 worsened these “stemness” signatures, restoring MYCT1 expression in cultured CB HSCs restored these dysregulated programs compared to control cells. Strikingly, maintaining MYCT1 expression improved ex vivo expansion of the most undifferentiated EPCR+ITGA3+ human HSPCs and enhanced engraftment ability upon transplantation to NSBGW mice. The inventors discovered that MYCT1 localizes in the endosomal membrane and interacts with vesicle trafficking and signaling machinery essential for HSC and EC function. Loss of MYCT1 led to hyperactivation of endocytosis and exaggerated signaling responses to cytokines in the culture microenvironment, whereas restoring MYCT1 expression in cultured human HSPCs was sufficient to balance abnormal endocytosis, improve stemness signatures that are disrupted in culture after MYCT1 KD, and restrain the excessive signaling responses. Strikingly, maintaining MYCT1 expression improved ex vivo expansion of the most undifferentiated EPCR+ITGA3+ human HSPC and enhanced engraftment ability upon transplantation to NSBGW mice. These data show that the moderation of environmental sensing in human HSCs through MYCT1-controlled endocytosis is essential for preserving human HSC stemness. As MYCT1 expression is downregulated in cultured HSPCs, the data imply that loss of the molecular machinery required for proper sensing of microenvironmental signals is a key contributor to the dysfunction of cultured human HSCs.

### Example 3: MYCT1 Controls Environmental Sensing in Human Hematopoietic Stem Cells

### Introduction

Hematopoietic stem cells (HSC) sustain the production of all blood lineages throughout life due to their unique ability to respond to microenvironmental cues to balance self-renewal and differentiation. Although HSCs can provide a life-saving therapy for patients with haematological malignancies and inherited blood disorders, access to HSC transplantation is limited by the difficulty in finding immune-compatible bone marrow donors and the low quantity of HSCs in cord blood¹⁻³. To address these bottlenecks, it has been a long-standing goal to expand human HSCs ex vivo^{4,5}. Despite the extensive efforts to improve the HSC culture environment, expansion of human HSCs that retain the capacity to self-renew and engraft upon transplantation has been challenging. Transfer of human HSCs from their niche to culture results in extensive changes in transcriptome, epigenome, signaling, metabolism, proteostasis and other cellular functions that severely compromise HSC self-renewal and engraftment ability^{6,7}. Multiple approaches have been taken to recapitulate human HSC self-renewal ex vivo⁷. These include co-culture with hematopoietic niche cells such as bone marrow mesenchymal stromal cells^{8,9} or endothelial cell (EC) lines¹⁰, 3D cultures within a hydrophilic matrix¹¹, supplementation of HSC cytokines with HSC supportive small molecules such as SR1¹² or UM171¹³, and most recently, albumin-free conditions replacing cytokines with chemical agonists¹⁴. Transcriptional regulators that are critical for human HSC identity and expansion ability, such as MLLT3¹⁵ and MSI2¹⁶, have been discovered and their function validated in human HSC expansion cultures. Another important goal is to identify biomarkers that reliably indicate preservation of HSC self-renewal ability in culture. Recent advances include improved surface markers for cultured human HSCs, such as EPCR¹⁷, ITGA3¹⁸, CD49f¹⁹ and RET²⁰, signature genes in HSC transcriptome (e.g. MLLT3¹⁵, HLF^{21,22}, MECOM²³), and characteristics that relate to maintaining cellular functions such as low mitochondrial activity and oxidative phosphorylation^{24,25}, proteostasis²⁶, or lysosomal function²⁷. Nevertheless, the majority of ex vivo expanded human hematopoietic cells that retain HSC immunophenotype are unable to repopulate the recipient hematopoietic system upon transplantation^{6,7}. Maximizing the yield of functional human HSCs in culture requires a better understanding of the biological processes that govern HSC self-renewal and engraftment ability and why these mechanisms become compromised in culture. Here, the inventors identify MYCT1 as a critical human HSC regulator that governs endocytosis in HSCs and moderates how HSCs sense microenvironmental signals. The aspects herein pinpoint silencing of MYCT1 expression in cultured human HSCs as a major contributor to the poor function of ex vivo expanded human HSCs and a biomarker for sustained human HSC function. MYCT1 expression is highly enriched in human HSC but lost during culture.

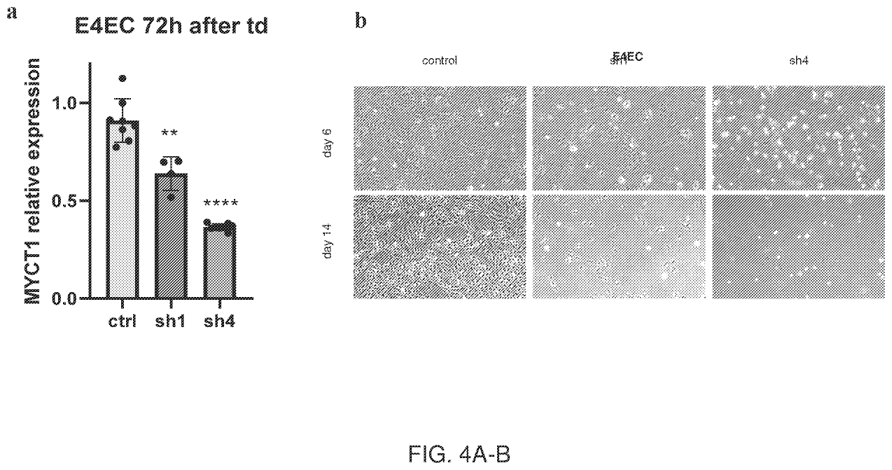

To uncover regulators of human HSC self-renewal that link to the poor function of cultured human HSCs, the inventors analysed RNA sequencing (RNAseq) datasets of human developmental and postnatal hematopoietic tissues and identified genes that are enriched in undifferentiated human HSPCs but become downregulated during culture. The inventors found a promising candidate, MYCT1 (Myc target 1, also known as MTLC), which is selectively expressed in endothelial cells (EC) and undifferentiated HSPC in all hematopoietic tissues and developmental stages, including 5-6 weeks aorta-gonad-mesonephros (AGM) region, placenta, yolk sac, 2^{nd} trimester foetal liver (FL), cord blood (CB) and adult bone marrow (BM) (FIG. 1a), as well as megakaryocytes²⁸. Moreover, MYCT1 levels were highest within the HSPC fractions enriched for the transplantable, self-renewing human HSCs in FL (GPI80+ fraction²⁹) and ex vivo expanded CB HSPCs (ITGA3+ fraction¹⁸) (FIG. 1b). Evaluation of the microarray herein and published microarray and RNA-seq datasets of human HSPCs cultured in various state-of-the-art HSC culture conditions and qPCR analysis of cultured CB HSPCs revealed that MYCT1 expression is highly sensitive to exposure to culture (FIG. 1c, Extended Data FIG. 1a-d)^{8,15,27}. Moreover, prolonged HSPC culture led to silencing of the active epigenetic marks at TSS and gene body that associate with MYCT1 expression in human HSPCs (Extended Data FIG. 1e). Our finding that MYCT1 expression is tightly linked to self-renewing human HSCs and its expression declines in culture concomitantly with the loss of engraftment ability suggest that MYCT1 may control critical functions in HSCs. 102 MYCT1 loss disrupts human HSPC expansion and engraftment ability to understand the functional consequences of MYCT1 loss on human HSCs, the inventors performed lentiviral shRNA-mediated knockdown (KD) on sorted human CB HSPCs using two different MYCT1 shRNAs and quantified their culture expansion and engraftment ability (FIG. 1d, Extended Data FIG. 2a,b). MYCT1 KD severely halted the ex vivo expansion of CB long-term (LT) HSCs (CD34+CD38−CD90+CD45RA−EPCR+ITGA3+), total HSPCs (CD34+CD38−CD90+CD45RA−), and their progeny. However, immunophenotypic LT-HSCs and HSPCs did not disappear from MYCT1 KD cultures (FIG. 1e, Extended Data FIG. 2c). Evaluation of HSPC division kinetics showed slowed cell division of MYCT1 KD HSPCs, suggesting a defect in their proliferative potential in vitro (Extended Data FIG. 3a,b). Importantly, when equal number of HSPCs sorted for the same surface phenotype (CD34+CD38−CD90+) were transplanted into NSG mice, MYCT1 KD completely abolished CB HSC engraftment ability, whereas control cells showed sustained multilineage engraftment (FIG. 1f,g,). MYCT1 KD in second trimester FL HSPCs also prevented HSC (CD34+CD38−CD90+GPI80+) expansion and generation of progeny in culture, and abrogated repopulation ability after transplantation (Extended Data FIG. 4a-d). These data identify MYCT1 as a novel human HSC regulator that is critically required for ex vivo expansion and transplantability across HSC ontogeny. 119 MYCT1 governs hallmarks of HSC functional competence

To decipher why MYCT1 is required for HSC expansion and engraftment ability, the inventors performed scRNAseq on uncultured and cultured CB HSPCs (CD34+CD38−CD90+) and evaluated the correlation of MYCT1 levels to HSC stemness programs within the most undifferentiated HLF+ HSCs^{21,22} (FIG. 2a). MYCT1 expressing uncultured HLF+ HSCs showed significantly higher expression of several genes associated with HSC identity and function (e.g. MLLT3, HIF1α, MEIS1) and lower expression of CDK6, a gene associated with ST-HSCs and HSC activation³⁰, as compared to HLF+MYCT1− and HLF− fractions (FIG. 2b). To investigate the immediate effects of MYCT1 loss, HSPCs from the same CB donor were transduced with MYCT1 KD vector after 24 h pre-stimulation and resorted (CD34+CD38−CD90+) 72 hours after transduction for scRNAseq. Since MYCT1 transcript levels decrease even in control cultures during this time, MYCT1 overexpression (OE) vector was used to rescue MYCT1 levels (FIG. 2a). MYCT1 KD and MYCT1 OE did not alter the proportion of HLF-expressing cells within the sorted HSPCs (Extended Data FIG. 5a-d). Despite their functional incompetence, MYCT1 KD HSCs retained even higher expression of several HSC identity genes (e.g. HLF, AVP, MECOM) than control HSCs. Nevertheless, expression of multiple human HSC “stemness” signature genes^{17,31-34} (e.g. MLLT3, HOXA9, PROM1) was downregulated in MYCT1 KD and upregulated in MYCT1 OE HLF+ HSCs, which typically showed a pattern more similar to uncultured HSCs (FIG. 2c). Functional enrichment analysis linked MYCT1 loss in HLF+ HSCs to a profound dysregulation of multiple cellular programs essential for HSC stemness, while MYCT1 OE had the opposite effect and showed closer similarity to uncultured HLF+ HSCs (FIG. 2d). MYCT1 loss triggered a rapid upregulation of genes involved in mitochondrial respiratory complexes and oxidative phosphorylation (Oxphos), which are also induced during culture-associated stress and have been linked to decreased HSC self-renewal ability^{24,25,35}. Indeed, FACS analysis verified increased mitochondrial membrane potential (TMRE) and mitochondrial reactive oxygen species (MitoSOX, indicative of mitochondrial activity) in MYCT1 KD HSPCs compared to control HSPCs, whereas MYCT1 OE suppressed these programs at transcriptional and cellular level (FIG. 2e-g). MYCT1 KD and OE also had opposing effects on the expression of ETS factors and multiple programs that govern HSC fate, such as spindle/M phase genes, splicing, ribosomal, and proteostasis programs³⁶⁻⁴⁰, with MYCT1 OE showing profiles more similar to uncultured cells (FIG. 2d and Extended Data FIG. 5c-j). These data indicate that the loss or gain of MYCT1 expression elicits major effects on programs regulating human HSC functional competence. Restoring MYCT1 expression improves human HSC function in culture.

Since MYCT1 expression is rapidly lost during human HSPC ex vivo culture and MYCT1 OE HSCs showed improved transcriptional profiles associated with HSC functional competency, the inventors investigated if maintaining MYCT1 expression during culture improves the function of ex vivo expanded human HSPCs (FIG. 3a). Lentiviral overexpression restored MYCT1 expression to levels comparable to uncultured HSPCs and resulted in prolonged culture maintenance and greater expansion of cells with LT-HSC surface phenotype (CD34+CD38−CD90+CD45RA−EPCR+ITAG3+). Total HSPC fraction (CD34+CD38−CD90+CD45RA−) and downstream HSPC/HPC populations (CD34+CD38−CD90−CD45RA− and CD34+CD38−) were also moderately expanded with MYCT1 OE (FIG. 3b, Extended Data FIG. 6a-c). Methylcellulose colony assays with MYCT1 OE HSPCs (CD34+CD38−CD90+) sorted 72-96 hours after transduction did not show a block in differentiation ability, but revealed a specific increase in the number of mixed (granulocyte, erythroid, macrophage) colonies that resulted in a modest increase in the total number of colonies. Furthermore, MYCT OE did not lead to excessive proliferation (Extended Data FIG. 6d-e). These data suggested that restoring MYCT1 levels during ex vivo culture improves the expansion of the most undifferentiated human CB HSPCs without disrupting the fate of their downstream progeny. Restoring MYCT1 expression improves human HSC engraftment ability.

The inventors next evaluated if the ex vivo expanded HSPCs where MYCT1 expression was rescued also performed better upon transplantation in vivo. Transplantation of equal numbers of immunophenotypic CB HSPCs (500 and 2500 CD34+CD38−CD90+ cells) into NBSGW mice 96 hours after transduction revealed more frequent BM engraftment with MYCT1 OE HSPCs, including multilineage and erythroid engraftment, 12 weeks after transplantation compared to control-transduced HSPCs. MYCT OE HSPCs also conferred higher total engraftment level of human hematopoietic cells (hCD45+) and HSPCs (CD34+CD38−) than control HSPCs did, and also gave rise to multilineage engraftment (FIG. 3c-e, Extended Data FIG. 7a,b). Limiting dilution analysis (LDA⁴¹) estimated a 2.17-fold improved frequency of engraftable HSCs 96 hours after transduction (1/1833 to 1/628 for MYCT1 OE vs 1/4310 to 1/1265 for control) (FIG. 3f).

To determine if MYCT1 OE HSPC cultured ex vivo over a longer period could sustain improved functionality compared to controls, the progeny of low to intermediate doses (50, 250, 500 and 2500 sorted GFP+ HSPCs) after 15 days in culture were transplanted. The frequency of engrafted mice was greater when the progeny of MYCT1 OE HSPCs was transplanted, and even the low doses (250, 500 cells) of MYCT1 OE HSPCs generated multilineage and erythroid engraftment in some mice. In the controls, only transplantation of the highest dose (2500 cells) resulted in multilineage engraftment, with no detectable erythroid engraftment (FIG. 3g,h, Extended Data 7c,d). LDA⁴¹ estimated a 12-fold increase in the frequency of engraftable HSCs with MYCT1 OE HSPCs cultured for 15 days (FIG. 3i). These data indicate that maintaining MYCT1 expression during ex vivo expansion improves the function of human HSPCs both in culture and in vivo, verifying the decline of MYCT1 expression as a significant contributor to the dysfunction of ex vivo expanded human HSPCs.

### Soluplus Based Human HSC Expansion Culture Conditions Improve MYCT1 Levels and MYCT1 Associated Programs

Culture conditions recently reported by Sakurai et al. that replace bovine serum albumin (BSA) with the caprolactam-based polymer Soluplus and HSC cytokines SCF and TPO with synthetic agonists (the PI3K activator 740Y-P and the TPO-receptor agonist butyzamide) and also include FLT3-L and small molecule UM171, promoted greater expansion of functional human CB HSPCs compared to using BSA, HSC cytokines and small molecules SR1 or UM171 alone¹⁴. Analysis of the Sakurai scRNAseq dataset revealed that their culture conditions (from here on called Soluplus media) not only increased the proportion of HLF+ HSCs within CD34+ cells in day 10 cultures as reported¹⁴, but also greatly improved MYCT1 expression and maintained MYCT1-expressing HLF+ HSC at a frequency similar to uncultured CB. In contrast, HLF+ HSCs cultured for 10 days in standard serum-free conditions with SR1 or UM171 showed minimal MYCT1 expression (Extended Data FIG. 8a-c). Furthermore, MYCT1 dependent programs revealed by MYCT1 KD and OE scRNAseq (ETS genes, mitochondrial and OXPHOS, spindle/M-phase, splicing, and proteostasis) were similarly improved in HSCs cultured with Soluplus media compared to SR1 or UM171 alone. Based on these key culture dysregulated programs, HSCs cultured for 10 days with Soluplus media were strikingly similar to MYCT1 OE HSCs cultured in standard conditions (Extended Data FIG. 8d-k). These data reinforce the concept that maintaining MYCT1 expression during HSC ex vivo culture contributes to improved HSC stemness programs and function, and suggest that MYCT1 expression can be used as an important biomarker to monitor functionally competent human HSCs in culture.

### MYCT1 is Localized in Endosomes

The structure and molecular function of MYCT1 are poorly defined, although there are reports suggesting that MYCT1 may act as a nuclear factor or as a membrane protein⁴²⁻⁴⁴. Based on its amino acid sequence, MYCT1 protein is predicted to have two transmembrane (TM) domains and a putative nuclear localization signal. Topology prediction⁴⁵ predicted the N- and C-terminal ends to be cytoplasmic, with a short non-cytoplasmatic region between the two TM domains (FIG. 4a). Evaluation of the localization of V5-tagged MYCT1 protein in KG1 cells (AML cells with human HSPC-like surface phenotype and gene expression programs, Extended Data FIG. 9a,b) revealed that MYCT1 is enriched in the membrane fraction and localizes in vesicle-like structures in the cytoplasm. Co-staining of V5-tagged MYCT1 with endosomal markers in KG1 cells and human CB HSPCs revealed MYCT1 co-localization with endosomal proteins, including clathrin (structurally responsible for the formation of coated vesicles in the initial steps of endocytosis), RAB5 (early endosomes), RAB7 (late endosomes), and RAB11 (late endosomes). No colocalization was found with a mitochondrial marker HSP60, implying the effects of MYCT1 KD and OE on mitochondrial activity are not due to MYCT1 expression in mitochondria (FIG. 4b-e). The co-localization with endosomal markers, together with the topology prediction, suggest that in HSCs, MYCT1 acts primarily in endosomes and localizes at the membrane of endosomes via the two transmembrane domains, with a short intra-endosomal loop (FIG. 4f)

### MYCT1 Interacts With Vesicle Trafficking and Signaling Proteins

To define the molecular function of MYCT1 the inventors performed immunoprecipitation coupled with high sensitivity mass spectrometry (IP-MS) of V5-tagged MYCT1 in hematopoietic and endothelial cell lines that also express MYCT1 endogenously. KG1 hematopoietic cells were chosen as they express HSC surface markers and many key HSC identity genes, and E4-immortalized HUVEC cells (E4EC)¹⁰ were chosen as they depend on MYCT1 expression for culture expansion (Extended Data FIG. 9a-d). MYCT1 was found to interact with proteins involved in vesicle trafficking and endosomes (e.g. early endosomal proteins RAB5A and C, clathrin adaptor AP2A1), cell surface receptor signaling and signal amplification (e.g. TGFBR1 and 2, NOTCH1 and 2, CTNNB1), G protein signaling, and cell adhesion, among others (FIG. 4g,h). MYCT1 interacting proteins identified in hematopoietic (KG1) and endothelial cells (E4EC) belong to the same families and are part of shared functional and/or physical networks (STRING⁴⁶), although the majority of interacting proteins were cell-type specific (FIG. 4h, Extended Data FIG. 9e-g).

To verify that MYCT1 interactors identified in KG1 and E4EC cell lines are relevant for the biology in primary human hematopoietic tissues, the inventors correlated the MYCT1 interactome with RNAseq data of MYCT1 expressing human HSPCs and their precursors from intra- and extraembryonic hemogenic tissues throughout ontogeny (see also FIG. 1a). The interactome genes from KG1 cells were typically expressed in 2^{nd} trimester FL, CB and adult BM HSPCs, whereas the MYCT1 interactors detected only in E4EC were expressed in ECs and newly emerged HSPCs from 1^{st} trimester hematopoietic tissues (5-6 weeks AGM, YS and PL) (Extended Data FIG. 9g). These data confirm that MYCT1 interacts in endosomes with vesicle trafficking and signaling proteins that are expressed at different stages of human HSC development and in ECs. These data also validate KG1 and E4EC as suitable models for studying MYCT1 function in human HSPCs throughout hematopoietic ontogeny.

### MYCT1 Controls Endocytosis and Environmental Sensing

Given the findings that MYCT1 localises in endosomal membrane and interacts with endosomal trafficking proteins, the inventors asked if MYCT1 regulates endocytosis. Quantification of internalization of fluorescent dextran in human CB HSPCs showed an increase in endocytosis after MYCT1 KD, while MYCT1 OE showed a decrease (FIG. 5a). MYCT1 KD in E4ECs also led to increased internalization of dextran (FIG. 5b). Monitoring of endocytosis in CB HSPC during culture showed increased endocytosis specifically in highly purified HSPCs (CD34+EPCR+) but not the differentiated progeny (CD34+EPCR− and CD34− cells). Importantly, restoring MYCT1 expression alone was sufficient to prevent the excessive culture induced endocytosis in HSPCs even after 10-12 days (FIG. 5c,d). These data show that MYCT1 is critical for controlling the rate of endocytosis in human HSCs.

Endocytosis is a critical regulatory step in extracellular signaling that can determine the length, strength, and quality of cell signaling from numerous receptors, and must be tightly controlled to maintain adequate responses to external signals⁴⁷⁻⁵⁰. To define if MYCT1 loss dysregulates signaling pathways, the inventors performed mass spectrometry-based global phospho-proteomic profiling in control and MYCT1 KD E4ECs (Extended Data FIG. 10a). MYCT1 KD led to global changes in the phospho-proteome of ECs, with increased phosphorylation of proteins involved in insulin, EGFR and Rho-GTPase signaling, as well as proteins involved in membrane trafficking and clathrin-mediated endocytosis (FIG. 5c, Extended Data FIG. 10b,c).

Phosphosite-centric predictions of relative kinase activity (KSEA⁵¹) and signature enrichment analysis (PTM-SEA⁵²) revealed signaling hyperactivation in response to MYCT1 loss. MYCT1 KD E4ECs showed increased predicted activity of receptor-mediated kinase signaling pathways, such as EGFR, INSR or KIT signaling, the downstream signal transduction pathways (PI3K-AKT, MAPKs such as ERK1/2), as well as endocytosis related kinases (CDK5, PAK1). In contrast, kinases and pathways involved in cell cycle progression and EC survival (CDC7 and PLK1 kinases, TIE2 and prolactin pathways) were inhibited upon MYCT1 loss. Importantly, MYCT1 KD E4ECs were enriched for signatures that have been observed in response to EGF and insulin treatment, although both control and KD cells are cultured in equal concentrations of EGF and IGF1 (insulin growth factor 1) (FIG. 5f,g). These data show that MYCT1 loss leads to broad hyperactivation of receptor-mediated kinase signaling pathways and dysregulation of other signaling networks critical for proliferation and survival, and suggest an increased sensitivity to the extracellular signals in the culture environment.

Western blot experiments in E4ECs further validated that MYCT1 KD leads to increased and prolonged responses to cytokine stimulation. Increased AKT and ERK phosphorylation, where multiple receptor signaling pathways converge, were observed in MYCT1 KD cells after stimulation with the standard cytokines present in the culture media (serum, EGF, IGF1, FGF). Even in starvation conditions (cytokine- and serum-starved overnight) MYCT1 KD led to greater AKT and ERK activation (Extended Data FIG. 10e-h). Accordingly, MYCT1 KD cells cultured in the continuous presence of serum and cytokines showed a constant basal hyperactivation of AKT and ERK compared to control cells (Extended Data FIG. 10i-k). Similarly, MYCT1 KD human CB HSPCs cultured in standard HSC supportive conditions (SFEM, SCF, FLT3, TPO with UM171 and SR1) showed AKT hyperactivation (FIG. 5h,i), which has been shown to be detrimental for HSC function^{53,54}.

Because endocytosis controls signaling from multiple receptors in different manners, the inventors interrogated single cell RNAseq data for responses to different signaling pathways. Module score analysis also reflected the hyperactivation of AKT signaling in MYCT1 KD HSPCs, and suggested decreased responses to TGFβ and NOTCH signaling and increased response to WNT signaling, all of which are known to be regulated by endocytosis⁴⁸, whereas rescue by MYCT1 OE had opposite effects on signaling responses (FIG. 5j). These data imply that MYCT1 controls environmental sensing in human HSCs to maintain HSC self-renewal and engraftment ability.

### Implications of Aspects Herein

The inventors discovered a critical role for HSC- and endothelial-specific gene MYCT1 in preserving human HSC self-renewal and engraftment ability during ex vivo culture due to its function in controlling endocytosis and environmental sensing. Loss of MYCT1 expression during human HSC culture led to hyperactivation of endocytosis and cytokine signaling, and complete abrogation of HSPC function. Maintaining MYCT1 expression during HSC culture alone was sufficient to dampen culture-induced excessive endocytosis and promote expansion of functional human HSCs, improving transplantation outcomes.

Endocytosis is classically viewed as the initial step in the endo-lysosomal pathway leading to signaling termination through degradation of membrane receptors, but it can also act as pro-signaling platform^{47,48,55,56}. The findings that hyperactivation of endocytosis in human HSCs following MYCT1 loss is coupled with excessive rather than dampened receptor-mediated signaling highlight the pro-signaling role of endocytosis in HSC biology, and suggest that HSCs require low endocytosis to maintain signaling responses within tolerable thresholds. Because endocytosis not only controls signaling from multiple signaling receptors, but also the membrane availability of nutrient receptors and ion channels (e.g. cholesterol and iron uptake)^{49,57}, dysregulation of endocytosis alters the responses to a myriad of environmental signals, as seen by the far-reaching effects induced by MYCT1 loss. These mechanisms together with hyperactive AKT signaling may be contributing to the increased mitochondrial activity, higher OXPHOS, and impaired HSC self-renewal associated with MYCT1 loss, and HSC culture more broadly. Despite endocytosis being an elemental process in all cell types, MYCT1 expression is selectively enriched in the most undifferentiated HSCs. Recent reports showed that endocytosis balances signaling cues to maintain pluripotency in embryonic stem cells^{60,61}, controls HSC emergence from hemogenic endothelium in zebrafish⁶², and BM homing in mice after transplantation⁶³, Interestingly, Myct1 and the vesicular trafficking regulator Gprasp2 were among 17 genes found in an shRNA screen to be essential for HSC repopulation in mice^{63,64}. The inventors hypothesize that, due to the intrinsic need of HSCs to switch between fates in a controlled, and in part reversible fashion, HSCs may require a stricter control of endocytosis and environmental sensing mechanisms to maintain “stemness”, compared to more differentiated cells. HSCs also require more precise control of mitochondrial activity^{24,35}, proteostasis^{26,65-67}, and lysosomal activity²⁷ than more differentiated cells and are less tolerant to imbalances that happen during stress, such as culture. Strikingly, many of these programs were found to be influenced by MYCT1. The discovery draws the attention to the essential cellular activities that HSCs must fine-tune to maintain their unique functional properties during ex vivo expansion, and uncovers MYCT1 as a critical regulatory node for these processes.

Despite the recent advances in understanding cellular and molecular mechanisms that are critical for maintaining HSC identity and self-renewal, the ability to control these processes ex vivo to improve HSC based therapeutic applications has been limited. This work highlights the importance of not just having the critical extrinsic signals in the culture environment, but also preserving the unique regulatory machinery in HSCs that can properly process these signals. Thus, improving the niche must be coupled with strategies aimed at maintaining the machinery responsible for environmental sensing, including MYCT1. The latest reported human HSC culture protocols that use Soluplus and chemical agonists to replace BSA, SCF, and TPO, and showed a greater expansion of functional human HSCs compared to other reported culture conditions¹⁴, also showed sustained MYCT1 expression. Although no mechanism for the improved function of Soluplus-expanded HSCs was reported, comparison of the Sakurai scRNAseq data to the MYCT1 KD and OE data showed similar correlation of MYCT1 expression with essential programs associated with HSC functional competence (e.g. expression of ETS factors, controlled mitochondrial metabolism and proteostasis). Future studies will be needed to understand why most culture conditions induce profound MYCT1 downregulation and epigenetic silencing of the MYCT1 TSS and active gene body marks. Identifying the upstream regulatory elements governing MYCT1 expression may provide key insights into the mechanisms underlying MYCT1 silencing and culture induced stress. This work nominates sustained MYCT1 expression and fine-tuned endocytosis as new hallmarks of human HSC quality control after ex vivo expansion.

### Example 4: Methods and Materials for Certain Aspects Herein

### Human HSPC Collection and Isolation

First trimester hematopoietic tissues and second trimester fetal livers (FL) (14-18 developmental weeks) were de-identified, discarded material obtained from elective terminations of pregnancy after informed consent. First trimester tissues (5-6 weeks) include the aorta-gonad-mesonephros (AGM) region, the placenta (PL), and the yolk sac (YS). Cord blood units were obtained at birth following informed consent and de-identified upon collection. Adult bone marrow (ABM) aspirates were purchased from Allcells. Because these tissues are discarded material with no personal identifiers, this research does not constitute human subjects research.

AGM, PL and YS were washed in Dulbecco's phosphate buffered saline (DPBS, Gibco 14190250), and placed in sterile DPBS with 5% FBS (FB-01 Omega scientific), 1% penicillin-streptomycin (Thermofisher Scientific 15140-122), and 2.5 μg/ml amphotericin B (Fisher BioReagents BP2645-50), and processed for sorting within 48 hours. Tissues were digested in 2.5 U dispase (Thermofisher Scientific 17105-041), 90 mg collagenase A (Worthington LS004176) and 0.075 mg DNase I (Sigma-Aldrich D4513) per ml in DPBS containing 10% FBS, for 20-45 min at 37° C. Cells were disaggregated by pipetting and filtered through a 70 μm cell strainer. FL were collected into DPBS with 5% FBS and mechanically dissociated using scalpels and syringes before proceeding with the enzymatic dissociation described above. Cord blood was diluted 1:2 with DPBS containing 2% FBS, 1 mM EDTA (Invitrogen AM9260G), and 4.2 U/ml DNAse I before proceeding to the enrichment of mononuclear cells.

For FL, CB, and ABM, mononuclear cells were enriched on SepMate-50 tubes using Lymphoprep (StemCell Technologies 85450 and 07861) layer following the manufacturer's protocol, and filtered through a 70 μm cell strainer. CD34+ cells were enriched using human CD34 MicroBead Kit UltraPure (Miltenyi Biotec 130-100-453). Cells were viably frozen or sorted directly.

### Cell Lines

Immortalized human umbilical vein endothelial cells (E4EC) (Butler et al. 2012) were obtained from Dr. Rafii's laboratory and cultured on gelatin-coated flasks with Medium 199 containing (GE Life Sciences sh30253.01) 20% FBS (FB-01 Omega scientific), 1% penicillin-streptomycin, 1% L-Glutamine, 10 mM HEPES (Thermofisher Scientific 15140-122, 2503-081, and 15630080), human FGF (20 ng/ml) (R&D systems 233-FB), human EGF (10 ng/ml), human IGF-I (10 ng/ml) (Peprotech AF-100-15 and 100-11), and heparin (50 μg/mL) (Sigma-Aldrich H3149-50KU). Human umbilical vein endothelial cells (HUVEC, Thermofisher Scientific C0035C) were cultured with Medium 200 supplemented with Low Serum Growth Supplement (LSGS) (Thermofisher Scientific M200500 and S00310) as described by the vendor. KG1 AML cells were cultured in RPMI with 10% FBS, 1% penicillin-streptomycin and 1% L-Glutamine.

### Cell Sorting and Flow Cytometry for HSC Assays

For identification and sorting of human HSPCs, single cell suspensions were stained with different combinations of antibodies against human CD34, CD38, CD90, GPI-80, EPCR, ITGA3, CD45RA. Dead cells were excluded with 7AAD or DAPI. Cells were assayed on a BD LSR Fortessa and analyzed with FlowJo software (Tree Star). Cell sorting was performed using a BD FACS Aria II.

### RNA-Sequencing for Human Hematopoietic Tissues and Cell Lines

For human hematopoietic tissues, cells were isolated as described above. The different populations were sorted directly into RLT based on surface markers. RNA was extracted using the RNeasy Micro kit (Qiagen 74004) and all RNA samples were sent to the CIRM consortium⁶⁸ for library preparation and sequencing. Sequencing libraries were prepared with the Ovation RNA-seq system V2 kit (NuGEN). Paired-end 150 bp sequencing was performed on the Illumina HiSeq 4000. Alignment to both the human genome (hg38) and the comprehensive genome annotation from Gencode (version 36) was performed using STAR⁶⁹. Data was normalized to fragments per kilobase of mappable length and million counts (FPKMs).

For KG1 and E4EC cell lines, total RNA was extracted using the RNeasy Mini kit (Qiagen 74104) and library was constructed using KAPA Stranded RNA-Seq Kit with RiboErase Kit. Different adapters were used for multiplexing samples in one lane. Sequencing was performed on Illumina HiSeq 3000 for SE 1×50 run. Data quality check was done on Illumina SAV. Demultiplexing was performed with Illumina Bcl2fastq v2.19.1.403 software. Alignment to the human genome (hg38) was performed using STAR⁶⁹. The hg38-Ensembl Transcripts release 101 gtf was used for gene feature annotation. Data was normalized to reads per kilobase million (RPKM).

Lentiviral vectors for MYCT1 shRNA-mediated knockdown and overexpression For MYCT1 knockdown shRNA experiments, pLKO lentiviral vectors from the MISSION TRC library (Millipore-Sigma) containing puromycin resistance gene with or without GFP were used: TRCN00000135691 (KD1), TRCN00000137125 (KD2), pLKO control (SHC001).

For MYCT1 overexpression, human MYCT1 was cloned from human FL HSPC full-length cDNA into the constitutive FUGW lentiviral vector (Addgene plasmid 14883, from D. Baltimore). MYCT1 cDNA with a C-terminal V5-tag was inserted downstream and in frame with the GFP sequence with the synthetic addition a P2A sequence between the 2 ORFs using PfuUltra II Fusion High Fidelity DNA polymerase (Agilent 600670).

Lentiviral production of shRNA and overexpression vectors and transduction For lentiviral production, 15-20 million 293T cells were plated with DMEM (Thermofisher Scientific 11995065) without antibiotics the day before transfection. Cells were transfected with 16 μg deltaR8.2 packaging plasmid, 8 μg VSVG-envelope plasmid, 20 μg of the lentiviral vector plasmid of choice and 132 μL of Turbo DNAfectin 3000 (Lambda Biotech G3000) in Opti-MEM (Thermofisher Scientific 31985070). 6-8 hours after transfection the media was changed to fresh complete DMEM. 72 hours after transfection the supernatant was filtered and concentrated by ultracentrifugation and pelleted viruses were resuspended in 200 μL SFEM or M199 (100× concentrated) and stored at −80° C.

HSPCs were thawed, sorted, and pre-stimulated for 24 hours before transduction. Transduction was performed with retronectin-bound spin infection. In short, non-treated plates were coated with Retronectin (Takara T100B) solution overnight at 4° C., blocked with 2% BSA in DPBS, and washed with DPBS. Virus (MOI 50-100) was added to the coated plate and centrifuged at 2,000×g for 2 hours at 32° C. The supernatant was removed, HSPCs were plated at a density of about 25,000 cells/cm² with Lentiboost (1:100) (Sirion Biotech SB-A-LF-900-01), and centrifuged again at 800×g for 90 minutes at room temperature. The culture media was changed the day after transduction. For shRNA lentiviral vectors, HSPCs were selected with puromycin (1 μg/ml) (Invivogen ant-pr-1), starting 24 hours after transduction. Endothelial cells were transduced by directly adding concentrated virus to cultured cells (10 μL/ml).

Assessment of MYCT1 expression by qRT-PCR during HSPC culture or after MYCT1 KD and OE RNA isolation was performed using the RNeasy Micro or Mini kit (Qiagen 74004 and 74104) with additional DNase step using manufacturer's protocol. cDNAs were prepared using High-Capacity cDNA Reverse Transcription Kit (Thermofisher Scientific 4368814), and qPCR for GAPDH and MYCT1 was performed with the LightCycler 480 SYBR Green I Master Mix (Roche 4707516001) on the Lightcycler 480 (Roche).

HSPC ex vivo culture and expansion assays Human HSPCs were cultured in serum-free conditions with StemSpan SFEM II (StemCell Technologies, 9655) supplemented with 1% Pen/Strep, 1% L-glutamine, human FLT3-L (100 ng/ml), human TPO (50 ng/ml), human SCF (100 ng/ml) (Thermofisher Scientific 15140122, 25030081, PHC9411, PHC9513, and PHC2113), human low-density lipoprotein (10 μg/mL), SR1 (500 nM) and UM171 (35 nM) (StemCell Technologies 2698, 72352, and 72914). Cells were cultured at 37° C. and 5% CO2, re-plated as necessary to maintain a cell density of ≤1×10⁶/mL, and half media changes were performed every other day. For HSPC expansion assays, cells were analysed by flow cytometry every 5-14 days. Absolute counting beads (Thermofisher Scientific, C36950) were used to determine cell numbers and calculate expansion rates. For co-culture with endothelial cells, E4EC were plated in standard media (M199 with serum and cytokines, see above) the day before adding HSPCs. When co-cultured with HSPCs, the standard supplemented StemSpan media was used.

Proliferation and cell cycle assays Dye dilution (CellTrace) proliferation assays and monitoring of single cell divisions were performed for control and MYCT1 KD or OE HSPCs 72-96 hours after transduction. To monitor single cell divisions, transduced HSPCs (CD34+CD38−CD90+) were sorted into single cells in 96-well round-bottom plates 72-96 hours after transduction. The cells in each well were visually counted every 24 hours for 96 hours.

For dye dilution proliferation assays, cells were resuspended at a concentration of 1×10⁶ cells/mL with warm DPBS with 5% BSA and CellTrace Violet (Thermofisher Scientific C34571) 5 μM and incubated at 37° C. for 15 minutes protected from light. The cells were then washed with 5× volume of cold DPBS 5% BSA, incubated for 5 minutes at 37° C., and resuspended in pre-warmed culture media and incubated for at least 10 minutes at 37° C. The cells were stained for HSC surface markers as described above and sorted for CD34+CD90+ and a high narrow peak of CellTrace Violet. Cells were analysed by FACS immediately after sorting and after 48 hours in culture.

Colony forming assays CB HSPCs were transduced with control or MYCT1 OE vectors. 72-96 hours after transduction, 180-300 sorted HSPCs (CD34+CD38−CD90+GFP+) were plated with methylcellulose-based medium containing recombinant cytokines for human cells (Methocult, StemCell Technologies H4435, contains SCF, IL3, IL6, EPO, G-CSF and GM-CSF) onto two 35 mm dishes (90-150 HSPCs per plate in duplicates). Colonies were counted and morphologically assessed after 15 days.

### HSPC Transplantation Assays After MYCT KD and FACS Quantification of Human Multilineage Engraftment

Engraftment ability of human MYCT1 KD and control HSPCs was assessed by transplanting equal numbers of sorted HSPCs into 9-11 weeks old immunodeficient NSG mice. Sorted foetal liver or cord blood CD34+CD38−CD90+ HSPCs were transduced 24 hours after sorting with MYCT1 shRNA (KD1) or control lentiviruses, selected with puromycin, re-sorted 72 hours after transduction and transplanted into female NSG mice. 10,000 HSPCs (CD34+CD38−CD90+GPI80+) from FL or 5,000 (CD34+CD38−CD90+) from CB were injected retro-orbitally into 6-16 weeks old female NSG mice (Jackson Laboratories). Mice were pre-conditioned by sub-lethal irradiation (2.75 Gy) 24 hours before transplantation or 25 mg/kg Busulfan treatment (intra-peritoneal injection) 24 and 48 hours before transplantation.

Human hematopoietic engraftment was quantified 6 and 12 weeks after transplantation by bone marrow biopsy. NSG mice from MYCT1 KD transplantation experiments were further analysed 24 weeks after transplantation by collecting cardiac blood, spleen, and bone marrow of euthanized mice. Human engraftment was evaluated by FACS after staining for human and mouse CD45, and multilineage engraftment was determined by the detection of human myeloid (CD14 or CD66b), B-lymphoid (CD19), and T-lymphoid/other (CD3, CD4 and/or CD8) markers within human CD45+ cells. The HSPC compartment was also evaluated using CD34 and CD38 markers.

### HSPC Transplantation Assays With MYCT Overexpression and FACS Quantification of Human Multilineage Engraftment

Engraftment ability of human MYCT1 overexpressing and control HSPCs was assessed by transplanting equal numbers of sorted HSPCs or their progeny into immunodeficient NBSGW mice. Cord blood HSPCs were transduced with MYCT1 OE or control lentiviral vectors, sorted and transplanted 96 hours after transduction into female or male 8-17 weeks old NBSGW mice (Jackson Laboratories) not requiring irradiation. 500 or 2,500 HSPCs (CD34+CD38−CD90+GFP+) were resuspended and injected as described above.

For the transplantation of HSPC progeny after 15 days in culture, sorted cord blood HSPCs were transduced with MYCT1 OE or control lentiviral vectors. Live GFP+ cells were re-sorted 72 hours after transduction, and further cultured for 10 additional days (total of 15 days in culture) and transplanted at limiting dilution doses. The number of cells reported (50, 250, 500, or 2,500 cells) is the number of sorted live GFP+ cells that were used to initiate the expansion cultures, the progeny of which was transplanted per mouse at day 15. 8-15 weeks old male and female NBSGW mice (Jackson Laboratories) not requiring irradiation were used as recipients.

For MYCT1 overexpression experiments, mice were considered to be engrafted if human CD45 was ≥0.1% of the total mouse and human hematopoietic compartment, and were considered to have multilineage engraftment if they displayed >0.01% of each myeloid (CD14 or CD66b), B-lymphoid (CD19), and T-lymphoid/other (CD3, CD4 and/or CD8) cells among the total mouse and human hematopoietic compartment. Erythroid engraftment was determined by the presence of CD71+GlyA+ cells. Frequency of reconstituting units was estimated from the total engraftment data with Extreme Limiting Dilution Analysis (ELDA⁴¹) including all mice from all replicates. All transplanted mice were included in the analysis unless they died before the particular experimental timepoint (1/9 mice transplanted with MYCT1 KD HSPC, 2/58 mice transplanted with control vector HSPC for the OE experiment). All studies and procedures involving mice were conducted in compliance with ethical regulations and were approved by the UCLA Animal Research Committee (protocol 2005-109).

### Single Cell RNAseq

CB HSPCs were sorted after thawing and sequenced directly (uncultured) or transduced with control, MYCT1 KD and MYCT1 OE vectors and re-sorted (CD34+CD38−CD90+GFP+) 72 hours after transduction. For single cell RNAseq, single cell suspensions in DPBS 0.04% Ultrapure BSA (Thermofisher Scientific AM2616) were used. A Chromium single cell instrument (10× genomics) was used for the generation of single-cell gel beads in emulsion. scRNA-seq libraries were prepared by using the Chromium single-cell 3′ library and gel bead kit v3 (10× Genomics). Sequencing was performed on the Illumina NovaSeq 6000 system. CellRanger mkfastq (v.2.1.1) was used to generate the fastq files, the CellRanger count was mapped to the human reference genome (refdata-cellranger-GRCh38-1.2.0), and the digital expression matrix was extracted from the ‘filtered_gene_bc_matrices’ folder output.

### Single Cell RNAseq Data Analysis

The single cell RNAseq data was analysed using the R package Seurat (v3.1.2). Cells with less than 100 unique molecular identifiers (UMIs) or more than 10% mitochondrial expression were removed from further analysis. Raw counts were normalized (Seurat function NormalizeData), variable genes identified (FindVariableGenes), expression values were scaled and centered in the dataset and the number of UMIs regressed against each gene (ScaleData). Principal component analysis (PCA), t-SNE and UMAP were used to reduce the dimensions of the data.

To investigate the differences between control, MYCT1 KD and MYCT1 OE HSCs, cells with more than 1 count for HLF were selected (HLF+), and the differentially expressed genes between HLF+ cells in the different samples (Ctrl, KD, OE) were obtained using the Seurat FindMarkers function using the DESeq2 test. Functional enrichment analysis was performed using both gProfiler⁷⁰ and PathfindR⁷¹. For gProfiler, the significant up- or downregulated genes (adjusted p value<0.05) were inputted ordered by Log FC and run as ordered query using the following sources: GO terms, KEGG signaling pathways, Reactome (REAC) and Wikipathway (WP), regulatory motif matches (TRANSFAC, TF), and protein complexes (CORUM). For PathfindR, all genes obtained from the Findmarkers analysis were inputted, including their Log FC and adjusted p value, and GO was used as source.

HLF+ cells from control, KD, and OE HSPCs were analysed separately from uncultured and HLF− negative cells by generating a Seurat subsample. The inventors compiled an HSC gene set by including human HSC-associated genes from the Fares dataset¹⁷, as well as the genes included in the curated HSC cell type signature gene sets from the GSEA database³¹: Descartes³², Hay³⁴, and Zheng³³ datasets. The final HSC gene set was matched with the lists of differentially expressed genes to determine if HSC-associated genes were differentially expressed in the HSC scRNAseq dataset. Gene modules were compiled for ETS transcription factors, and for the relevant functional categories by including all genes belonging to the particular GO term (obtained from QuickGO⁷², https://www.ebi.ac.uk/QuickGO/annotations). Scores were calculated using AddModuleScore with default parameters, and significance was calculated using Wilcoxon rank sum test. The module scores and the expression patterns of selected genes are shown using the DotPlot function.

MYCT1 regulated programs from MYCT1 OE and KD data sets were compared to scRNA data of cultured human HSCs published by Sakurai et al. (GEO; GSE192519)¹⁴. This data set compared human cord blood HSPCs expanded for 10 days in new Soluplus conditions (culture conditions that replace albumin with caprolactam-based polymer Soluplus, and HSC cytokines SCF and TPO with synthetic agonists, i.e. the PI3K activator 740Y-P and the TPO-receptor agonist butyzamide, and also include FLT3L and UM171), to HSPCs expanded in standard HSC cytokines (SCF, FLT3, and TPO, with IL6 included only for the SR1 conditions) with UM171 or SR1 alone. Seurat objects were generated for each sample using Read10X and CreateSeuratObject functions. Cells that had unique feature counts of over 7,500 or less than 200 were filtered out, as were cells with mitochondrial counts higher than 10%. Raw counts were normalized, variable genes identified, data scaled and PCA, t-SNE and UMAP run as described above. HSCs were selected based on HLF expression (>1 count) and Seurat subsamples were generated. The subsamples containing the HSCs from each culture condition (herein called Soluplus, UM171, or SR1) were integrated using SelectIntegrationFeatures with n=3000 features, FindIntegrationAnchors with and k.filter=90 (due to the low number of HSC in the SR1 sample), and the IntegrateData function. Data was then scaled (ScaleData). PCA, t-SNE and UMAP were used to reduce the dimensions of the data. Module scores were calculated for the integrated Seurat sample and plotted using AddModuleScore and DotPlot as performed for MYCT1 KD and OE samples.

### FACS Based Mitochondrial Assays

Mitochondrial membrane potential and mitochondrial ROS were quantified using TMRE (Abcam, ab113852) and MitoSOX (Thermofisher Scientific, M36008) respectively. HSPCs transduced with control, KD or OE lentiviral vectors were incubated for 20 minutes at 37° C. with 50 nM of TMRE or 5 μM of MitoSOX. Because HSCs can efflux MitoSOX dyes, 50 μM of verapamil was added for the incubation⁷³. DAPI was included to discriminate dead cells.

### FACS Based Endocytosis Assays

HSPC or endothelial cells transduced with control, MYCT1 KD, or MYCT1 OE lentiviral vectors were incubated with fluorescent dextran (pHrodo Red Dextran, ThermoFisher Scientific P10361) 40 μg/ml for 30 minutes at 37° C. After incubation, cells were immediately placed on ice, washed with ice-cold DPBS with 5% FBS and resuspended into single cell suspension for FACS analysis. 7AAD was included to discriminate dead cells.

### Cell Fractionation

Cell fractionation was performed on 2×10⁶ KG1 cells overexpressing MYCT1-V5 using the Subcellular Protein Fractionation Kit for Cultured Cells (Thermofisher Scientific 78840) and following the manufacturer's instructions with addition of wash steps in between fractions.

### Immunofluorescence

KG1 cells overexpressing MYCT1-V5 were spun on slides at 200×g for 10 minutes, fixed with 4% paraformaldehyde (Electron Microscopy Sciences 15710) for 10 minutes, permeabilized with 0.1% Triton X-100 (Sigma T9284) for 5 minutes, rinsed twice with DPBS, and blocked (DPBS with 0.5% BSA, 5% donkey serum (Jackson Immunoresearch 017-000-121), 5% goat serum (Abcam ab7481), 0.1% Triton) for 30 minutes. Primary antibodies were prepared in blocking solution and incubated overnight at 4° C. Slides were washed 4 times with permeabilizing solution and secondary antibodies prepared in blocking buffer were incubated for 1 hour at room temperature. Slides were washed 4 times more, stained with DAPI (Miltenyi Biotec 130-111-570) solution (1:1000) for 10 minutes, rinsed with DPBS, and mounted with ProLong Gold antifade mountant (Thermofisher Scientific P10144). Images were acquired with a Zeiss LSM880 confocal at 40×, and processed and analysed for colocalization using Imaris v9.7.2. For immunofluorescence in CB HSPCs transduced with MYCT1 OE vector, GFP positive cells were sorted 72 hours after transduction and spun on poly-lysine (Sigma-Aldrich P8920) coated slides. Staining was performed the same way, with the addition of ImageIT signal enhancer (Thermofisher Scientific I36933) for 30 minutes at room temperature before the blocking step.

### MYCT1-V5 Immunoprecipitation (IP)

KG1 or E4EC were washed twice with ice cold DPBS and lysed with RIPA buffer containing protease inhibitors (Thermofisher Scientific 78429) by rotating the tubes for 30 minutes. Cell debris was removed by centrifugation for 15 minutes at 14.000×g and collecting the supernatant. Protein concentration was quantified by BCA. 1-2 mg of protein, together with 1-2 mg of protein G beads, and 4-8 μg of V5 antibody (Thermofisher Scientific 10004D and R960-25) were incubated overnight with rotation. The beads were washed 3 times with wash buffer (150 mM NaCl, 50 nM Tris pH 7.5) with 0.5% NP-40 and 5 times with wash buffer without NP-40 for 10 minutes each. Beads were eluted in 80 μL urea 8M in 100 mM Tris-HCl pH 8 digestion buffer by shaking at 25° C. for 30 minutes. All steps including centrifugation were performed at 4° C. unless otherwise stated. If used for western blot, the proteins were eluted in RIPA buffer (Sigma-Aldrich R0278) with Laemli (Bio-Rad 1610747) containing beta-mercaptoethanol (Thermofisher Scientific 21985023), and 4% of the total lysate was used for input

If the samples were used for mass spectrometry, protein disulfide bonds from the eluted IP samples were subjected to reduction using 5 mM Tris (2-carboxyethyl) phosphine for 30 min, free cysteine residues were alkylated by 10 mM iodoacetamide for another 30 min. Samples were diluted with 100 mM Tris-HCl at pH 8 to reach a urea concentration of less than 2 M then digested sequentially with Lys-C and trypsin at a 1:100 protease-to-peptide ratio for 3 and 18 hours, respectively. After addition of formic acid to 5% (vol/vol), samples were desalted using C18 tips (Thermofisher Scientific 87784) and dried in a SpeedVac vacuum concentrator and reconstituted in 5% formic acid for LC-MS/MS processing.

### Phospho-Proteomics in E4EC After MYCT1 KD

E4ECs were grown in 15 cm dishes, transduced with control or MYCT1 KD vectors (5 dishes per condition), and selected with puromycin. 72 hours after transduction the cells were washed twice with cold DPBS, and collected by scraping. The whole cell pellets were washed twice more and resuspended in 150 μl digestion buffer of 8 M urea, 100 mM Tris-HCl pH 8, 1 mM MgCl2, 100 μL each of phosphatase inhibitor cocktails (Abcam ab201112), and protease inhibitors (GoldBio AEBSF, Pepstatin A GoldBio P-020-25, Leupeptin GoldBio L-010-25) followed by reduction, alkylation and drying as described above. For enrichment of phosphorylated peptides, the dried peptides were enriched using Fe-NTA enrichment columns (Thermofisher Scientific A32992) before LC-MS/MS processing.

### Mass Spectrometry LC-MS/MS Processing and Data Quantification

The peptide mixtures were loaded onto a 25 cm long, 75 μm inner diameter fused-silica capillary, packed in-house with bulk 1.9 μM ReproSil-Pur beads with 120 Å pores as described previously⁷⁴. Peptides were analysed using a 140 min water-acetonitrile gradient delivered by a Dionex Ultimate 3000 UHPLC (Thermo Fisher Scientific) operated initially at 400 nL/min flow rate with 1% buffer B (acetonitrile solution with 3% DMSO and 0.1% formic acid) and 99% buffer A (water solution with 3% DMSO and 0.1% formic acid). Buffer B was increased to 6% over 5 min at which time the flow rate was reduced to 200 nl/min. A linear gradient from 6-28% B was applied to the column over the course of 123 min. The linear gradient of buffer B was further increased to 28-35% for 8 min followed by a rapid ramp-up to 85% for column washing. Eluted peptides were ionized via a Nimbus electrospray ionization source (Phoenix S&T) by application of a distal voltage of 2.2 kV. All label-free mass spectrometry data were collected using data dependent acquisition on Orbitrap Fusion Lumos Tribrid mass spectrometer (Thermo Fisher Scientific) with an MS1 resolution of 120,000 followed by sequential MS2 scans at a resolution of 15,000. Label-free quantitation was performed using the MaxQuant software package⁷⁵. The EMBL Human reference proteome (UP000005640 9606) was utilized for all database searches. Statistical analysis of MaxQuant output data was performed with the artMS Bioconductor package (version 1.4.2) which performs the relative quantification of protein abundance using the MSstats Bioconductor package (default parameters). Intensities were normalized across samples by median-centering the log 2-transformed MS1 intensity distributions. The abundance of proteins missing from one condition but found in more than 2 biological replicates of the other condition for any given comparison were estimated by imputing intensity values from the lowest observed MS1-intensity across samples and p-values were randomly assigned to those between 0.05 and 0.01 for illustration purposes.

### Data Analysis of MYCT1 Interacting Proteins From IP-Mass Spectrometry

For the interactome IP-MS, the results were first filtered using the Contaminant Repository for Single Epitope tag IP-MS⁷⁶, and proteins enriched in all biological replicates of MYCT1-V5 samples compared to the controls were selected. To generate the protein-protein association networks, STRING⁴⁶ version 11.5 was used, with high confidence settings and k means clustering (https://string-db.org/cgi/input). Gene ontology, pathway analysis and CORUM protein complex analysis was performed using gProfiler⁷⁰ (https://biit.cs.ut.ee/gprofiler/gost).

### Data Analysis for Phospho-Proteomics

For the phospho proteomics experiment, protein-centric pathway analysis (Reactome, WikiPathways, KEGG) was performed using gProfiler⁷⁰ for the proteins with increased or decreased phosphorylation (Log 2FC≥0.58 or ≤−0.58, 1.5-fold and 0.66-fold respectively, and P value<0.05). Site-centric relative kinase activity prediction was performed with KSEA⁵¹ (https://casecpb.shinyapps.io/ksea) using all the identified phospho-sites as input and the following settings: dataset from PhosphoSitePlus+NetworkKIN, NetworkKIN score cutoff=2. Site-centric pathway and perturbation analysis was performed using PTM-SEA⁵² version PTMsigDB v1.9.0 with the default parameters and a minimum overlap of 2 for pathways or 5 for perturbations.

### Western Blot for Signaling Activation

E4EC transduced with control or MYCT1 KD vectors were collected 72 hours after transduction or starved overnight for 16 hours by replacing the regular E4EC growth media (see “Cell lines”) with starvation media (E4EC growth media without FBS, FGF, EGF and IGF-1), and re-stimulated with regular growth media containing serum and cytokines for the indicated time points, which were made to coincide with 72 hours since transduction. Cord blood HSPCs were lysed 72-96 hours after transduction with control, MYCT1 KD, or MYCT1 OE vectors. Cells were lysed in RIPA buffer containing protease and phosphatase inhibitors and protein quantification was performed using BCA protein quantification kit (Thermofisher Scientific 78440, and 23227). The lysates were prepared with Laemli containing beta-mercaptoethanol and the proteins were denatured for 10 minutes at 95° C. Approximately 4 μg of protein were loaded per well. Western blot images were acquired using a BioRad Chemidoc Touch Imaging System and quantified using ImageLab 6.0.1.

### REFERENCES

1. Ballen, K. K., Gluckman, E. & Broxmeyer, H. E. Umbilical cord blood transplantation: the first 25 year and beyond. Blood 122, 491-498 (2013).
2. Gragert, L. et al. HLA Match Likelihoods for Hematopoietic Stem-Cell Grafts in the U.S. Registry. N Engl J Med 371, 339-348 (2014).
3. Ng, A. P. & Alexander, W. S. Haematopoietic stem cells: past, present and future. Cell Death Discovery 3, 17002 (2017).
4. Pineault, N. & Abu-Khader, A. Advances in umbilical cord blood stem cell expansion and clinical translation. Experimental Hematology 43, 498-513 (2015).
5. Cohen, S. et al. Hematopoietic stem cell transplantation using single UM171-expanded cord blood: a single-arm, phase 1-2 safety and feasibility study. The Lancet Haematology 7, e134-e145 (2020).
6. Dahlberg, A., Delaney, C. & Bernstein, I. D. Ex vivo expansion of human hematopoietic stem and progenitor cells. Blood 117, 6083-6090 (2011).
7. Fares, I., Calvanese, V. & Mikkola, H. K. A. Decoding Human Hematopoietic Stem Cell Self-Renewal. Curr Stem Cell Rep (2022) doi:10.1007/s40778-022-00209-w.
8. Magnusson, M. et al. Expansion on Stromal Cells Preserves the Undifferentiated State of Human Hematopoietic Stem Cells Despite Compromised Reconstitution Ability. PLOS ONE 8, e53912 (2013).
9. Nakahara, F. et al. Engineering a haematopoietic stem cell niche by revitalizing mesenchymal stromal cells. Nat Cell Biol 21, 560-567 (2019).
10. Butler, J. M. et al. Development of a vascular niche platform for expansion of repopulating human cord blood stem and progenitor cells. Blood 120, 1344-1347 (2012).
11. Bai, T. et al. Expansion of primitive human hematopoietic stem cells by culture in a zwitterionic hydrogel. Nat Med 25, 1566-1575 (2019).
12. Boitano, A. E. et al. Aryl Hydrocarbon Receptor Antagonists Promote the Expansion of Human Hematopoietic Stem Cells. Science 329, 1345-1348 (2010)
13. Fares, I. et al. Pyrimidoindole derivatives are agonists of human hematopoietic stem cell self-renewal. Science 345, 1509-1512 (2014).
14. Sakurai, M. et al. Chemically defined cytokine-free expansion of human haematopoietic stem cells. Nature 615, 127-133 (2023).
15. Calvanese, V. et al. MLLT3 governs human haematopoietic stem-cell self-renewal and engraftment. Nature 576, 281-286 (2019).
16. Rentas, S. et al. Musashi-2 attenuates AHR signaling to expand human haematopoietic stem cells. Nature 532, 508-511 (2016).
17. Fares, I. et al. EPCR expression marks UM171-expanded CD34+ cord blood stem cells. Blood 129, 3344-3351 (2017).
18. Tomellini, E. et al. Integrin-α3 Is a Functional Marker of Ex Vivo Expanded Human Long-Term Hematopoietic Stem Cells. Cell Reports 28, 1063-1073.e5 (2019).
19. Notta, F. et al. Isolation of Single Human Hematopoietic Stem Cells Capable of Long-Term Multilineage Engraftment. Science 333, 218-221 (2011).
20. Grey, W. et al. Activation of the receptor tyrosine kinase RET improves long-term hematopoietic stem cell outgrowth and potency. Blood 136, 2535-2547 (2020).
21. Lehnertz, B. et al. HLF expression defines the human hematopoietic stem cell state. Blood 138, 2642-2654 (2021).
22. Calvanese, V. et al. Mapping human haematopoietic stem cells from haemogenic endothelium to birth. Nature 604, 534-540 (2022).
23. Goyama, S. et al. Evi-1 Is a Critical Regulator for Hematopoietic Stem Cells and Transformed Leukemic Cells. Cell Stem Cell 3, 207-220 (2008).
24. Vannini, N. et al. Specification of haematopoietic stem cell fate via modulation of mitochondrial activity. Nat Commun 7, 13125 (2016).
25. Papa, L. et al. Limited Mitochondrial Activity Coupled With Strong Expression of CD34, CD90 and EPCR Determines the Functional Fitness of ex vivo Expanded Human Hematopoietic Stem Cells. Front Cell Dev Biol 8, 592348 (2020).
26. Chua, B. A. et al. Hematopoietic stem cells preferentially traffic misfolded proteins to aggresomes and depend on aggrephagy to maintain protein homeostasis. Cell Stem Cell 30, 460-472.e6 (2023).
27. García-Prat, L. et al. TFEB-mediated endolysosomal activity controls human hematopoietic stem cell fate. Cell Stem Cell 28, 1838-1850.e10 (2021).
28. Novershtern, N. et al. Densely Interconnected Transcriptional Circuits Control Cell States in Human Hematopoiesis. Cell 144, 296-309 (2011).
29. Prashad, S. L. et al. GPI-80 defines self-renewal ability in hematopoietic stem cells during human development. Cell Stem Cell 16, 80-87 (2015).
30. Laurenti, E. et al. CDK6 Levels Regulate Quiescence Exit in Human Hematopoietic Stem Cells. Cell Stem Cell 16, 302-313 (2015).
31. Subramanian, A. et al. Gene set enrichment analysis: A knowledge-based approach for interpreting genome-wide expression profiles. Proceedings of the National Academy of Sciences 102, 15545-15550 (2005).
32. Cao, J. et al. A human cell atlas of fetal gene expression. Science 370, eaba7721 (2020).
33. Zheng, S., Papalexi, E., Butler, A., Stephenson, W. & Satija, R. Molecular transitions in early progenitors during human cord blood hematopoiesis. Mol Syst Biol 14, e8041 (2018).
34. Hay, S. B., Ferchen, K., Chetal, K., Grimes, H. L. & Salomonis, N. The Human Cell Atlas bone marrow single-cell interactive web portal. Exp Hematol 68, 51-61 (2018).
35. Filippi, M.-D. & Ghaffari, S. Mitochondria in the maintenance of hematopoietic stem cells: new perspectives and opportunities. Blood 133, 1943-1952 (2019).
36. Zimdahl, B. et al. Lis1 regulates asymmetric division in hematopoietic stem cells and in leukemia. Nat 921 Genet 46, 245-252 (2014).
37. Chan, D. C. H. et al. Arhgef2 regulates mitotic spindle orientation in hematopoietic stem cells and is essential for productive hematopoiesis. Blood Advances 5, 3120-3133 (2021).
38. Chen, L. et al. Transcriptional diversity during lineage commitment of human blood progenitors. Science 345, 1251033 (2014).
39. Lv, K. et al. HectD1 controls hematopoietic stem cell regeneration by coordinating ribosome assembly and protein synthesis. Cell Stem Cell 28, 1275-1290.e9 (2021).
40. Signer, R. A. J., Magee, J. A., Salic, A. & Morrison, S. J. Haematopoietic stem cells require a highly regulated protein synthesis rate. Nature 509, 49-54 (2014).
41. Hu, Y. & Smyth, G. K. ELDA: Extreme limiting dilution analysis for comparing depleted and enriched populations in stem cell and other assays. Journal of Immunological Methods 347, 70-78 (2009).
42. Yin, X., Grove, L., Rogulski, K. & Prochownik, E. V. Myc target in myeloid cells-1, a novel c-Myc target, recapitulates multiple c-Myc phenotypes. J. Biol. Chem. 277, 19998-20010 (2002).
43. Rogulski, K. R., Cohen, D. E., Corcoran, D. L., Benos, P. V. & Prochownik, E. V. Deregulation of common genes by c-Myc and its direct target, MT-MC1. Proceedings of the National Academy of Sciences 102, 18968-18973 (2005).
44. Wu, S. et al. Transmembrane domain is crucial to the subcellular localization and function of Myc target 1. Journal of Cellular and Molecular Medicine 20, 471-481 (2016).
45. Käll, L., Krogh, A. & Sonnhammer, E. L. L. Advantages of combined transmembrane topology and signal peptide prediction—the Phobius web server. Nucleic Acids Research 35, W429-W432 (2007).
46. Szklarczyk, D. et al. STRING v11: protein-protein association networks with increased coverage, supporting functional discovery in genome-wide experimental datasets. Nucleic Acids Research 47, D607-D613 (2019).
47. Murphy, J. E., Padilla, B. E., Hasdemir, B., Cottrell, G. S. & Bunnett, N. W. Endosomes: A legitimate platform for the signaling train. Proceedings of the National Academy of Sciences 106, 17615-17622 (2009).
48. Sorkin, A. & von Zastrow, M. Endocytosis and signaling: intertwining molecular networks. Nat Rev Mol 948 Cell Biol 10, 609-622 (2009).
49. Polo, S. & Fiore, P. P. D. Endocytosis Conducts the Cell Signaling Orchestra. Cell 124, 897-900 (2006).
50. Villaseñor, R., Kalaidzidis, Y. & Zerial, M. Signal processing by the endosomal system. Current Opinion in Cell Biology 39, 53-60 (2016).
51. Wiredja, D. D., Koyutürk, M. & Chance, M. R. The KSEA App: a web-based tool for kinase activity inference from quantitative phosphoproteomics. Bioinformatics 33, 3489-3491 (2017).
52. Krug, K. et al. A Curated Resource for Phosphosite-specific Signature Analysis *[S]. Molecular & Cellular Proteomics 18, 576-593 (2019).
53. Chen, C. et al. TSC-mTOR maintains quiescence and function of hematopoietic stem cells by repressing mitochondrial biogenesis and reactive oxygen species. Journal of Experimental Medicine 205, 2397-2408 (2008).
54. Wu, F., Chen, Z., Liu, J. & Hou, Y. The Akt-mTOR network at the interface of hematopoietic stem cell homeostasis. Experimental Hematology 103, 15-23 (2021).
55. Irannejad, R. & von Zastrow, M. GPCR signaling along the endocytic pathway. Current Opinion in Cell 963 Biology 27, 109-116 (2014).
56. Di Fiore, P. P. & von Zastrow, M. Endocytosis, Signaling, and Beyond. Cold Spring Harb Perspect Biol 6, a016865 (2014).
57. Antonescu, C. N., McGraw, T. E. & Klip, A. Reciprocal Regulation of Endocytosis and Metabolism. Cold Spring Harb Perspect Biol 6, a016964 (2014).
58. Morganti, C. & Ito, K. Mitochondrial Contributions to Hematopoietic Stem Cell Aging. International Journal of Molecular Sciences 22, 11117 (2021).
59. Papa, L., Djedaini, M. & Hoffman, R. Mitochondrial Role in Stemness and Differentiation of Hematopoietic Stem Cells. Stem Cells International 2019, e4067162 (2019).
60. Narayana, Y. V., Gadgil, C., Mote, R. D., Rajan, R. & Subramanyam, D. Clathrin-Mediated Endocytosis Regulates a Balance between Opposing Signals to Maintain the Pluripotent State of Embryonic Stem Cells. Stem Cell Reports 12, 152-164 (2019).
61. Sangokoya, C. & Blelloch, R. MicroRNA-dependent inhibition of PFN2 orchestrates ERK activation and pluripotent state transitions by regulating endocytosis. Proceedings of the National Academy of Sciences 117, 20625-20635 (2020).
62. Heng, J. et al. Rab5c-mediated endocytic trafficking regulates hematopoietic stem and progenitor cell development via Notch and AKT signaling. PLOS Biology 18, e3000696 (2020).
63. Morales-Hernandez, A. et al. GPRASP proteins are critical negative regulators of hematopoietic stem cell transplantation. Blood 135, 1111-1123 (2020).
64. Holmfeldt, P. et al. Functional screen identifies regulators of murine hematopoietic stem cell repopulation. The Journal of Experimental Medicine 213, 433-449 (2016).
65. Chua, B. A. & Signer, R. A. J. Hematopoietic stem cell regulation by the proteostasis network. Current Opinion in Hematology 27, 254 (2020).
66. Rehn, M. et al. PTBP1 promotes hematopoietic stem cell maintenance and red blood cell development by ensuring sufficient availability of ribosomal constituents. Cell Reports 39, 110793 (2022).
67. Zhao, J. et al. Human hematopoietic stem cell vulnerability to ferroptosis. Cell 186, 732-747.e16 (2023).
68. Sun, V. et al. The Metabolic Landscape of Thymic T Cell Development In Vivo and In Vitro. Frontiers in Immunology 12, (2021).
69. Dobin, A. et al. STAR: ultrafast universal RNA-seq aligner. Bioinformatics 29, 15-21 (2013).
70. Raudvere, U. et al. g:Profiler: a web server for functional enrichment analysis and conversions of gene lists (2019 update). Nucleic Acids Research 47, W191-W198 (2019).
71. Ulgen, E., Ozisik, O. & Sezerman, O. U. pathfindR: An R Package for Comprehensive Identification of Enriched Pathways in Omics Data Through Active Subnetworks. Frontiers in Genetics 10, (2019).
72. Binns, D. et al. QuickGO: a web-based tool for Gene Ontology searching. Bioinformatics 25, 3045-3046 (2009).
73. Mansell, E. et al. Mitochondrial Potentiation Ameliorates Age-Related Heterogeneity in Hematopoietic Stem Cell Function. Cell Stem Cell 28, 241-256.e6 (2021).
74. Jami-Alahmadi, Y., Pandey, V., Mayank, A. K. & Wohlschlegel, J. A. A Robust Method for Packing High Resolution C18 RP-nano-HPLC Columns. JoVE 62380 (2021) doi:10.3791/62380.
75. Cox, J. & Mann, M. MaxQuant enables high peptide identification rates, individualized p.p.b.-range mass accuracies and proteome-wide protein quantification. Nat Biotechnol 26, 1367-1372 (2008).
76. Mellacheruvu, D. et al. The CRAPome: a contaminant repository for affinity purification-mass spectrometry data. Nat Methods 10, 730-736 (2013).
77. Barretina, J. et al. The Cancer Cell Line Encyclopedia enables predictive modelling of anticancer drug sensitivity. Nature 483, 603-607 (2012).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for enhancing hematopoietic stem cell function, the method comprising introducing a nucleic acid encoding MYCT1 into a hematopoietic stem cell.

2. The method of claim 1, wherein the hematopoietic stem cell is a human hematopoietic stem cell.

3. The method of claim 1 or 2, further comprising culturing the hematopoietic stem cell for at least 24 hours.

4. The method of any one of claims 1-3, further comprising introducing the hematopoietic stem cell into a subject.

5. The method of claim 4, wherein the subject is a human subject.

6. The method of claim 4 or 5, wherein the hematopoietic stem cell engrafts into bone marrow of the subject.

7. The method of any one of claims 1-6, further comprising differentiating the hematopoietic stem cell into a blood cell.

8. The method of any one of claims 1-7, wherein the nucleic acid molecule is a plasmid.

9. The method of any one of claims 1-7, wherein the nucleic acid molecule is a vector.

10. The method of claim 9, wherein the vector is a viral vector.

11. The method of claim 10, wherein the viral vector is a recombinant adeno-associated viral vector, a recombinant adenoviral vector, a recombinant lentiviral vector, or a recombinant retroviral vector.

12. The method of any one of claims 1-7, wherein the nucleic acid is a DNA molecule.

13. The method of any one of claims 1-7, wherein the nucleic acid is an mRNA molecule.

14. The method of any one of claims 1-13, wherein introducing the nucleic acid comprises transfection.

15. The method of any one of claims 1-13, wherein the nucleic acid molecule does not integrate into the genome of the hematopoietic stem cell.

16. The method of any one of claims 1-12, wherein the nucleic acid molecule integrates into the genome of the hematopoietic stem cell.

17. The method of any one of claims 1-16, wherein the method comprises introducing a nucleic acid molecule encoding MYCT1 into each of a population of hematopoietic stem cells.

18. The method of any one of claims 1-17, further comprising introducing a nucleic acid encoding MLLT3 into the hematopoietic stem cell.

19. A modified hematopoietic stem cell having increased expression or activity of MYCT1 relative to an unmodified hematopoietic stem cell.

20. The modified hematopoietic stem cell of claim 19, wherein the hematopoietic stem cell comprises an exogenous nucleic acid molecule encoding MYCT1.

21. The modified hematopoietic stem cell of claim 19, wherein the hematopoietic stem cell comprises a genetic modification that increases the expression of MYCT1.

22. The modified hematopoietic stem cell of claim 19, wherein the hematopoietic stem cell comprises a genetic modification that increases the activity of MYCT1.

23. A method for enhancing hematopoietic stem cell function, the method comprising subjecting a hematopoietic stem cell to conditions sufficient to inhibit endocytosis.

24. The method of claim 23, wherein the conditions comprise administration of an endocytosis inhibitor.

25. The method of claim 23, wherein the conditions increase expression or activity of MYCT1.

26. The method of claim 25, wherein the conditions comprise introducing a nucleic acid encoding MYCT1 into a hematopoietic stem cell.

27. The method of claim 26, wherein the nucleic acid molecule is a plasmid.

28. The method of claim 26, wherein the nucleic acid molecule is a vector.

29. The method of claim 28, wherein the vector is a viral vector.

30. The method of claim 29, wherein the viral vector is a recombinant adeno-associated viral vector, a recombinant adenoviral vector, a recombinant lentiviral vector, or a recombinant retroviral vector.

31. The method of claim 26, wherein the nucleic acid is an mRNA molecule.

32. The method of any one of claims 26-31, wherein introducing the nucleic acid comprises transfection.

33. The method of any one of claims 26-31, wherein the nucleic acid molecule does not integrate into the genome of the hematopoietic stem cell.

34. The method of any one of claims 26-28, wherein the nucleic acid molecule integrates into the genome of the hematopoietic stem cell.

35. The method of any one of claims 26-34, wherein the method comprises introducing a nucleic acid molecule encoding MYCT1 into each of a population of hematopoietic stem cells.

36. The method of any one of claims 26-35, further comprising introducing a nucleic acid encoding MLLT3 into the hematopoietic stem cell.
